SEQUENCE LISTING (40 sequences)

SEQ ID NO: 1 - DNA, 5896 bp, Homo sapiens
ctgcggccggcccgcgagctaggctgggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtatggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggctcttctgagcttacgtagttctaccggctttgccgtgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggaaaaaaaaaaaaaaaa

SEQ ID NO: 2 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 3 - DNA, 1071 bp, Homo sapiens
atgaatggggtctcggaggggaccagaggctgcagtgacaggcaacctggggtcctgacacgtgatcgctcttgttccaggaagatgaactcttccggatgcctgtctgaggaggtggggtccctccgcccactgactgtggttatcctgtctgcgtccattgtcgtcggagtgctgggcaatgggctggtgctgtggatgactgtcttccgtatggcacgcacggtctccaccgtctgcttcttccacctggcccttgccgatttcatgctctcactgtctctgcccattgccatgtactatattgtctccaggcagtggctcctcggagagtgggcctgcaaactctacatcacctttgtgttcctcagctactttgccagtaactgcctccttgtcttcatctctgtggaccgttgcatctctgtcctctaccccgtctgggccctgaaccaccgcactgtgcagcgggcgagctggctggcctttggggtgtggctcctggccgccgccttgtgctctgcgcacctgaaattccggacaaccagaaaatggaatggctgtacgcactgctacttggcgttcaactctgacaatgagactgcccagatttggattgaaggggtcgtggagggacacattatagggaccattggccacttcctgctgggcttcctggggcccttagcaatcataggcacctgcgcccacctcatccgggccaagctcttgcgggagggctgggtccatgccaaccggcccaagaggctgctgctggtgctggtgagcgctttctttatcttctggtccccgtttaacgtggtgctgttggtccatctgtggcgacgggtgatgctcaaggaaatctaccacccccggatgctgctcatcctccaggctagctttgccttgggctgtgtcaacagcagcctcaaccccttcctctacgtcttcgttggcagagatttccaagaaaagtttttccagtctttgacttctgccctggcgagggcgtttggagaggaggagtttctgtcatcctgtccccgtggcaacgccccccgggaatga

SEQ ID NO: 4 - protein, 356 aa, Homo sapiens
MNGVSEGTRGCSDRQPGVLTRDRSCSRKMNSSGCLSEEVGSLRPLTVVILSASIVVGVLGNGLVLWMTVFRMARTVSTVCFFHLALADFMLSLSLPIAMYYIVSRQWLLGEWACKLYITFVFLSYFASNCLLVFISVDRCISVLYPVWALNHRTVQRASWLAFGVWLLAAALCSAHLKFRTTRKWNGCTHCYLAFNSDNETAQIWIEGVVEGHIIGTIGHFLLGFLGPLAIIGTCAHLIRAKLLREGWVHANRPKRLLLVLVSAFFIFWSPFNVVLLVHLWRRVMLKEIYHPRMLLILQASFALGCVNSSLNPFLYVFVGRDFQEKFFQSLTSALARAFGEEEFLSSCPRGNAPRE

SEQ ID NO: 5 - DNA, 2574 bp, Homo sapiens
cctggcgcagccgcagcagcgacgcgagcgaactcggccgggcccgggcgcgcgggggcgggacgcgcacgcggcgagggcggcgggtgagccgggggcggggacgggggcgggacgggggcgaagggggcggggacgggggcgcccgccggcctaacgggattaggagggcgcgccacccgcttccgctgcccgccggggaatcccccgggtggcgcccagggaagttcccgaacgggcgggcataaaagggcagccgcgccggcgccccacagctctgcagctcgtggcagcggcgcagcgctccagccatgtcgcgcggcctccagcttctgctcctgagctgcgcctacagcctggctcccgcgacgccggaggtgaaggtggcttgctccgaagatgtggacttgccctgcaccgccccctgggatccgcaggttccctacacggtctcctgggtcaagttattggagggtggtgaagagaggatggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactcctcacaagacagaactggtatgagcaggatttctgcaggttcttcttcctgaagctgaggctcaggggtgtgcctgtctgttacactggaggagagaagaatgagcctacgctgaagatggcatcctgtgaagtccttcacctcactgaaaacatctggaaggggatcccaccccattttctgtgggcaggcctcgaaaaccatcacatgaccacatagcatgaggccactgctgcttctccatggccaccttttcagcgatgtatgcagctatctggtcaacctcctggacattttttcagtcatataaaagctatggtgagatgcagctggaaaagggtcttgggaaatatgaatgcccccagctggcccgtgacagactcctgaggacagctgtcctcttctgcatcttggggacatctctttgaattttctgtgttttgctgtaccagcccagatgttttacgtctgggagaaattgacagatcaagctgtgagacagtgggaaatatttagcaaataatttcctggtgtgaaggtcctgctattactaaggagtaatctgtgtacaaagaaataacaagtcgatgaactattccccagcagggtcttttcatctgggaaagacatccataaagaagcaataaagaagagtgccacatttatttttatatctatatgtacttgtcaaagaaggtttgtgtttttctgcttttgaaatctgtatctgtagtgagatagcattgtgaactgacaggcagcctggacatagagagggagaagaagtcagagagggtgacaagatagagagctatttaatggccggctggaaatgctgggctgacggtgcagtctgggtgctcgcccacttgtcccactatctgggtgcatgatcttgagcaagttccttctggtgtctgctttctccattgtaaaccacaaggctgttgcatgggctaatgaagatcatatacgtgaaaattatttgaaaacatataaagcactatacagattcgaaactccattgagtcattatccttgctatgatgatggtgttttggggatgagagggtgctatccatttctcatgttttccattgtttgaaacaaagaaggttaccaagaagcctttcctgtagccttctgtaggaattcttttggggaagtgaggaagccaggtccacggtctgttcttgaagcagtagcctaacacactccaagatatggacacacgggagccgctggcagaagggacttcacgaagtgttgcatggatgttttagccattgttggctttcccttatcaaacttgggcccttcccttcttggtttccaaaggcatttattgctgagttatatgttcactgtccccctaatattagggagtaaaacggataccaagttgatttagtgtttttacctctgtcttggctttcatgttattaaacgtatgcatgtgaagaagggtgtttttctgttttatattcaactcataagactttgggataggaaaaatgagtaatggttactaggcttaatacctgggtgattacataatctgtacaacgaacccccatgatgtaagtttacctatgtaacaaacctgcacttatacccatgaacttaaaatgaaagttaaaaataaaaaacatatacaaataaaaaaaa

SEQ ID NO: 6 - protein, 205 aa, Homo sapiens
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 7 - DNA, 1067 bp, Homo sapiens
cggctcaagtgaactgactctgctagaacagtgccgtgcttttccacagaaggttagaccctgaaagagatggctcagcaccacctatggatcttgctcctttgcctgcaaacctggccggaagcagctggaaaagactcagaaatcttcacagtgaatgggattctgggagagtcagtcactttccctgtaaatatccaagaaccacggcaagttaaaatcattgcttggacttctaaaacatctgttgcttatgtaacaccaggagactcagaaacagcacccgtagttactgtgacccacagaaattattatgaacggatacatgccttaggtccgaactacaatctggtcattagcgatctgaggatggaagacgcaggagactacaaagcagacataaatacacaggctgatccctacaccaccaccaagcgctacaacctgcaaatctatcgtcggcttgggaaaccaaaaattacacagagtttaatggcatctgtgaacagcacctgtaatgtcacactgacatgctctgtagagaaagaagaaaagaatgtgacatacaattggagtcccctgggagaagagggtaatgtccttcaaatcttccagactcctgaggaccaagagctgacttacacgtgtacagcccagaaccctgtcagcaacaattctgactccatctctgcccggcagctctgtgcagacatcgcaatgggcttccgtactcaccacaccgggttgctgagcgtgctggctatgttctttctgcttgttctcattctgtcttcagtgtttttgttccgtttgttcaagagaagacaagatgctgcctcaaagaaaaccatatacacatatatcatggcttcaaggaacacccagccagcagagtccagaatctatgatgaaatcctgcagtccaaggtgcttccctccaaggaagagccagtgaacacagtttattccgaagtgcagtttgctgataagatggggaaagccagcacacaggacagtaaacctcctgggacttcaagctatgaaattgtgatctaggctgctgggct

SEQ ID NO: 8 - protein, 328 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQDAASKKTIYTYIMASRNTQPAESRIYDEILQSKVLPSKEEPVNTVYSEVQFADKMGKASTQDSKPPGTSSYEIVI

SEQ ID NO: 9 - DNA, 1561 bp, Homo sapiens
tccacccaagagcaacctggaactaagttattcggcaacgaactgttccactttgttgtgaggcaatagatgtggaaattccctgacgaggggctctgtcctcatacttcctgcggagcttattgtcgtaagaatatctgtcatcctgctaatgtgcattgaaaggagagcaacggggctgaggccgtgtcagcacgatggaccccaaacagaccaccctcctgtgtcttggggactttcccatgcctttcatatctgccaaatcgagtcctgtgattcccttggatggatctgtgaaaatccagtgccaggccattcgtgaagcttacctgacccagctgatgatcataaaaaactccacgtaccgagagataggcagaagactgaagttttggaatgagactgatcctgagttcgtcattgaccacatggacgcaaacaaggcagggcgctatcagtgccaatataggatagggcactacagattccggtacagtgacaccctggagctggtagtgacaggcttgtatggcaaacccttcctctctgcagatcggggtctggtgttgatgccaggagagaatatttccctcacgtgcagctcagcacacatcccatttgatagattttcactggccaaggagggagaactttctctgccacagcaccaaagtggggaacacccggccaacttctctttgggtcctgtggacctcaatgtctcagggatctacagactccatccaccaagattacacgacgcagaacttgatccgcatggccgtggcaggactggtcctcgtggctctcttggccatactggttgaaaattggcacagccatacggcactgaacaaggaagcctcggcagatgtggctgaaccgagctggagccaacagatgtgtcagccaggattgacctttgcacgaacaccaagtgtctgcaagtaaacacctggaggtgaaggcagagaggagccaggactgtggagtccgacaaagctacttgaaggacacaagagagaaaagctcactaagaagcttgaatctacttttttttttttttgagacagagtctggctctgtcacccaggctgaagtgcagtggagcaatctcggctcattgaacctcttgggttcaagtgattcttgtgcctcagcctcccaagtagctggaattacaggcacataccactgcacccagctaatttttgtatttttagtagagatggggtttcactgtgttggccaggctggtctcgaactcctgacctcaggtgatccacccaccttggcctcccaaagtgctgagattataggcatgagccaccacgcctggccagatgcatgttcaaaccaatcaaatggtgttttcttatgcaggactgatcgatttgcacccacctttctgcacataagttatggttttccatcttatctgtcttctgattttttatatcctgtttaatttcttccttcattgttcttctctttttttatttattttatttatttttatttttatttttatttgagacagagtctcactctgttgcccagg

SEQ ID NO: 10 - protein, 209 aa, Homo sapiens
MDPKQTTLLCLGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGELSLPQHQSGEHPANFSLGPVDLNVSGIYRLHPPRLHDAELDPHGRGRTGPRGSLGHTG

SEQ ID NO: 11 - DNA, 2202 bp, Homo sapiens
ggaaacatgatccagctgaaggactgattgcaggaaaacttggcagctccccaaccttggtggcccagggagtgtgaggctgcagcctcagaaggtgtgagcagtggccacgagaggcaggctggctgggacatgaggttggcagagggcaggcaagctggcccttggtgggcctcgccctgagcactcggaggcactcctatgcttggaaagctcgctatgctgctgtgggtccagcaggcgctgctcgccttgctcctccccacactcctggcacagggagaagccaggaggagccgaaacaccaccaggcccgctctgctgaggctgtcggattaccttttgaccaactacaggaagggtgtgcgccccgtgagggactggaggaagccaaccaccgtatccattgacgtcattgtctatgccatcctcaacgtggatgagaagaatcaggtgctgaccacctacatctggtaccggcagtactggactgatgagtttctccagtggaaccctgaggactttgacaacatcaccaagttgtccatccccacggacagcatctgggtcccggacattctcatcaatgagttcgtggatgtggggaagtctccaaatatcccgtacgtgtatattcggcatcaaggcgaagttcagaactacaagccccttcaggtggtgactgcctgtagcctcgacatctacaacttccccttcgatgtccagaactgctcgctgaccttcaccagttggctgcacaccatccaggacatcaacatctctttgtggcgcttgccagaaaaggtgaaatccgacaggagtgtcttcatgaaccagggagagtgggagttgctgggggtgctgccctactttcgggagttcagcatggaaagcagtaactactatgcagaaatgaagttctatgtggtcatccgccggcggcccctcttctatgtggtcagcctgctactgcccagcatcttcctcatggtcatggacatcgtgggcttctacctgccccccaacagtggcgagagggtctctttcaagattacactcctcctgggctactcggtcttcctgatcatcgtttctgacacgctgccggccactgccatcggcactcctctcattggtgtctactttgtggtgtgcatggctctgctggtgataagtttggccgagaccatcttcattgtgcggctggtgcacaagcaagacctgcagcagcccgtgcctgcttggctgcgtcacctggttctggagagaatcgcctggctactttgcctgagggagcagtcaacttcccagaggcccccagccacctcccaagccaccaagactgatgactgctcagccatgggaaaccactgcagccacatgggaggaccccaggacttcgagaagagcccgagggacagatgtagccctcccccaccacctcgggaggcctcgctggcggtgtgtgggctgctgcaggagctgtcctccatccggcaattcctggaaaagcgggatgagatccgagaggtggcccgagactggctgcgcgtgggctccgtgctggacaagctgctattccacatttacctgctagcggtgctggcctacagcatcaccctggttatgctctggtccatctggcagtacgcttgagtgggtacagcccagtggaggagggggtacagtcctggttaggtggggacagaggatttctgcttaggcccctcaggacccagggaatgccagggacattttcaagacacagacaaagtcccgtgccctgtttccaatgccaattcatctcagcaatcacaagccaaggtctgaacccttccaccaaaaactgggtgttcaaggcccttacacccttgtcccacccccagcagctcaccatggctttaaaacatgctctcttagatcaggagaaactcgggcactccctaagtccactctagttgtggacttttccccattgaccctcacctgaataagggactttggaattctgcttctctttcacaactttgcttttaggttgaaggcaaaaccaactctctactacacaggcctgataactctgtacgaggcttctctaacccctagtgtcttttttttcttcacctcacttgtggcagcttccctgaacactcatcccccatcagatgatgggagtgggaagaataaaatgcagtgaaaccc

SEQ ID NO: 12 - protein, 478 aa, Homo sapiens
MLLWVQQALLALLLPTLLAQGEARRSRNTTRPALLRLSDYLLTNYRKGVRPVRDWRKPTTVSIDVIVYAILNVDEKNQVLTTYIWYRQYWTDEFLQWNPEDFDNITKLSIPTDSIWVPDILINEFVDVGKSPNIPYVYIRHQGEVQNYKPLQVVTACSLDIYNFPFDVQNCSLTFTSWLHTIQDINISLWRLPEKVKSDRSVFMNQGEWELLGVLPYFREFSMESSNYYAEMKFYVVIRRRPLFYVVSLLLPSIFLMVMDIVGFYLPPNSGERVSFKITLLLGYSVFLIIVSDTLPATAIGTPLIGVYFVVCMALLVISLAETIFIVRLVHKQDLQQPVPAWLRHLVLERIAWLLCLREQSTSQRPPATSQATKTDDCSAMGNHCSHMGGPQDFEKSPRDRCSPPPPPREASLAVCGLLQELSSIRQFLEKRDEIREVARDWLRVGSVLDKLLFHIYLLAVLAYSITLVMLWSIWQYA

SEQ ID NO: 13 - DNA, 1425 bp, Homo sapiens
cagtctgagaacaagaaagaagaacttctgtctcgagggtctcactgtcaaccaggccagagtgcagtgaagatcatacctcactacatccgtgaactcccgggctcctcccacctaagtctcttgagtagctgggacttcaggagactgaagccaaggataccagcagagccaacatttgcttcaagttcctgggcctgctgacagcgtgcaggatgctgttggaacccggcagaggctgctgtgccctggccatcctgctggcaattgtggacatccagtctggtggatgcattaacatcaccagctcagcttcccaggaaggaacgcgactaaacttaatctgtactgtatggcataagaaagaagaggctgaggggtttgtagtgtttttgtgcaaggacaggtctggagactgttctcctgagaccagtttaaaacagctgagacttaaaagggatcctgggatagatggtgttggtgaaatatcatctcagttgatgttcaccataagccaagtcacaccgttgcacagtgggacctaccagtgttgtgccagaagccagaagtcaggtatccgccttcagggccattttttctccattctattcacagagacagggaactacacagtgacgggattgaaacaaagacaacaccttgagttcagccataatgaaggcactctcagttcaggcttcctacaagaaaaggtctgggtaatgctggtcaccagccttgtggcccttcaagctttgtaagcctgtccaaaagaacttttaaaacagctacagcaagatgagtctgactatggcttagtatctttctcattacaataggcacagagaagaatgcaacagggcacaggggaagagatgctaaatataccaagaatctgtggaaatataagctggggcaaatcagtgtaatccttgactttgctcctcaccatcagggcaaacttgccttcttccctcctaagctccagtaaataaacagaacagctttcaccaaagtgggtagtatagtcctcaaatatcggataaatatatgcgtttttgtaccccagaaaaacttttcctccctcttcatcaacatagtaaaataagtcaaacaaaatgagaacaccaaattttgggggaataaatttttatttaacactgcaaaggaaagagagagaaaacaagcaaagataggtaggacagaaaggaagacagccagatccagtgattgacttggcatgaaaatgagaaaatgcagacagacctcaacattcaacattcaacaacatccatacagcactgctggaggaagaggaagatttgtgcagaccaagagcaccacagactacaactgcccagcttcatctaaatacttgttaacctctttggtcatttctctttttaaataaatgcccatagcagt

SEQ ID NO: 14 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 15 - DNA, 4775 bp, Homo sapiens
acccgcgcgaggtaggcgctctggtgcttgcggaggacgcttccttcctcagatgcaccgatcttcccgatactgcctttggagcggctagattgctagccttggctgctccattggcctgccttgccccttacctgccgattgcatatgaactcttcttctgtctgtacatcgttgtcgtcggagtcgtcgcgatcgtcgtggcgctcgtgtgatggccttcgtccgtttagagtagtgtagttagttaggggccaacgaagaagaaagaagacgcgattagtgcagagatgctggaggtggtcagttactaagctagagtaagatagcggagcgaaaagagccaaacctagccggggggcgcacggtcacccaaaggaggtcgactcgccggcgcttcctatcgcgccgagctccctccattcctctccctccgccgaggcgcgaggttgcggcgcgcagcgcagcgcagctcagcgcaccgactgccgcgggctccgctgggcgattgcagccgagtccgtttctcgtctagctgccgccgcggcgaccgctgcctggtcttcctcccggacgctagtgggttatcagctaacacccgcgagcatctataacataggccaactgacgccatccttcaaaaacaactaaaggatgatatgatgaacctagcctgttaatttcgtcttctcaattttaaactttggttgcttaagactgaagcaatcatggtgaacctgaggaatgcggtgcattcattccttgtgcacctaattggcctattggtttggcaatgtgatatttctgtgagcccagtagcagctatagtaactgacattttcaatacctccgatggtggacgcttcaaattcccagacggggtacaaaactggccagcactttcaatcgtcatcataataatcatgacaataggtggcaacatccttgtgatcatggcagtaagcatggaaaagaaactgcacaatgccaccaattacttcttaatgtccctagccattgctgatatgctagtgggactacttgtcatgcccctgtctctcctggcaatcctttatgattatgtctggccactacctagatatttgtgccccgtctggatttctttagatgttttattttcaacagcgtccatcatgcacctctgcgctatatcgctggatcggtatgtagcaatacgtaatcctattgagcatagccgtttcaattcgcggactaaggccatcatgaagattgctattgtttgggcaatttctataggtgtatcagttcctatccctgtgattggactgagggacgaagaaaaggtgttcgtgaacaacacgacgtgcgtgctcaacgacccaaatttcgttcttattgggtccttcgtagctttcttcataccgctgacgattatggtgattacgtattgcctgaccatctacgttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagcttcgaaagtccttgggattgttttctttgtgtttctgatcatgtggtgcccatttttcattaccaatattctgtctgttctttgtgagaagtcctgtaaccaaaagctcatggaaaagcttctgaatgtgtttgtttggattggctatgtttgttcaggaatcaatcctctggtgtatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtgagaaagaacagcacagtcttttcctacggtacaagctacatatgtaggaaaattttcttctttaatttttctgttggtcttaactaatgtaaatattgctgtctgaaaaagtgtttttacatatagctttgcaaccttgtactttacaatcatgcctacattagtgagatttagggttctatatttactgtttataataggtggagactaacttattttgattgtttgatgaataaaatgtttatttttgctctccctcccttctttccttccttttttcctttcttccttcctttctctctttcttttgtgcatatggcaacgttcatgttcatctcaggtggcatttgcaggtgaccagaatgaggcacatgacagtggttatatttcaaccacacctaaattaacaaattcagtggacatttgttctgggttaacagtaaatatacactttacattcttgctctgctcatctacacatataaacacagtaagataggttctgctttctgatacatctgtcagtgagtcagaggcagaacctagtcttgttgttcatataggggcaaaaatttgacattgtcagaatgttgtgttggtatttactgcaatgtctgtccctaaacatagtggtattttaacatagcagctggttaaccgggactacagaagtggaaggataatgagatgtaatacaccaaatagcttttcacttcttaaggacagtgttcaaattctgattattacaacaagcaaactgaaattagtgttttcattctggtccttagtaaattcctaattctatgattaaactgggaaatgagatcccagagttatttcccaacccaggattcaacatcaattgggttttgatctcagcatcctggaaatttgtgtgcttcacacaaagtgaaattagtattttgagccttattaaaatattttcttaattatggtacctctgtctataggacttaatttagcagtccatttttgagtaaaacttgtattggaagtatagatggtagaaactttggaagttttacttgattaaggactacagaattgggcccttagaatgtgaaaaaaaaaagtaattaaaaagacacttttaccgaactcgggattacagaaacacggagtttccatttggattttaaacaaaatttatgtcattttcagatccttccaaactctctagtgcaggaaaaggctgcagctaatttgtgaaagtggcaagctcttcattgcactgcagttatttaccagaagtttaaatctttgttaaaatatagtgttgtgttacaataagtgttggccatcatttcattcgtgggcctgctgctctctaagaattcagtagcattttaatagtttctaaaccatgaaaagttttcaagcattgctaaagtcaggccattcagtctatgctgtgtgcagagtatacaagtgtttctagtaacagtatttccatacgtgcccatttcacacaactgtggataaattttggaagaattcatgatgctagttcttacgcttgacagttacttacacacctgagaatgtgcctctcagtatcttaaaattggttaatgaaaaatctgaatttctaaaacccttggtctgtgttctcaacacacagtatagataaatccaatagtctgccacaagggcagtggaagagctgctgtatttgaggaaactcatacagtctctatttgatttgcaacactggccaaacatcagtcatttgcttgagcatgcccaaatattacatgaaagtcaagtctacctgccttgcctgttaggtctgttgaagtgcatgttaaaataattatatgaagcagaatgagatgatttaattcttaccgaaatgaaaatggctgaagaaacacagcatgcatttagcatgagttctgcacatacagatggtgtcctgcatgtatgccatgtatgttgcatgaatccatcgatttgtattaatgtagggcagaatagctgatagaagaaggactgaagaaaatccttcagcaatccttaaaaagaccatgcattcagatctgaagtagtgtgagtgttagaaaaaactggaaacatctgatttctgaactatcagggcaagctcatagcacatgttttacaaagaaacaaaatataaatcacagatttccaaaagtactagcaataagttgaatgataatagctcacagcacatttgttaatgattcttgtgtcatcaagtagtagtacttaatagtacccaacctggtaattatcctcaagttgtgtgctattcgtaagttctgtgcagtttggtatgaaacaaatatactcatttggatataaatcttacccttcaatgttaaatctacaaacttttataaatgttttaaagaagtccatgtgataattgtaaaggtgatgaatttaccatcaaacaaatcattttgatgtattattatatatgtatatctgtgtaagacacgtgcaacagactgccttatattattttctgtaattcttctcctttgtcaaatggtattttttgtgaatggttgcaaagtgttgtcttattcctaattcctgtatgttatccactacaggttttatgagacttcctattaatttattaaatttattaaatgttgaaaaaaaaaaaaaaaaaaaaaaa

SEQ ID NO: 16 - protein, 458 aa, Homo sapiens
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 17 - DNA, 1892 bp, Homo sapiens
tggagccatgctccctgggctcttccgcgggcgcccgcgcgctgcccttcgcttgaggcaaaaggactcttgtggaagatggaactcattgtccattttccagaatgtatttccaagcccatcaatgggacctgatactgctgttctgtgttgaaatgcttgaagaactcctgcatctctgcttgcatcttccatcctactgaaaccatggtcttctcggcagtgttgactgcgttccataccgggacatccaacacaacatttgtcgtgtatgaaaacacctacatgaatattacactccctccaccattccagcatcctgacctcagtccattgcttagatatagttttgaaaccatggctcccactggtttgagttccttgaccgtgaatagtacagctgtgcccacaacaccagcagcatttaagagcctaaacttgcctcttcagatcaccctttctgctataatgatattcattctgtttgtgtcttttcttgggaacttggttgtttgcctcatggtttaccaaaaagctgccatgaggtctgcaattaacatcctccttgccagcctagcttttgcagacatgttgcttgcagtgctgaacatgccctttgccctggtaactattcttactacccgatggatttttgggaaattcttctgtagggtatctgctatgtttttctggttatttgtgatagaaggagtagccatcctgctcatcattagcatagataggttccttattatagtccagaggcaggataagctaaacccatatagagctaaggttctgattgcagtttcttgggcaacttccttttgtgtagcttttcctttagccgtaggaaaccccgacctgcagataccttcccgagctccccagtgtgtgtttgggtacacaaccaatccaggctaccaggcttatgtgattttgatttctctcatttctttcttcatacccttcctggtaatactgtactcatttatgggcatactcaacacccttcggcacaatgccttgaggatccatagctaccctgaaggtatatgcctcagccaggccagcaaactgggtctcatgagtctgcagagacctttccagatgagcattgacatgggctttaaaacacgtgccttcaccactattttgattctctttgctgtcttcattgtctgctgggccccattcaccacttacagccttgtggcaacattcagtaagcacttttactatcagcacaacttttttgagattagcacctggctactgtggctctgctacctcaagtctgcattgaatccgctgatctactactggaggattaagaaattccatgatgcttgcctggacatgatgcctaagtccttcaagtttttgccgcagctccctggtcacacaaagcgacggatacgtcctagtgctgtctatgtgtgtggggaacatcggacggtggtgtgaatattggaactggctgacattttgggtgatgcttgttctttattgacattgaattctctttctcatagcctctccactttatttttttttatagggtttgtgtatgtatgtgtgtgagcagtgtaaagaaagaatggtaattatagttctgttaccaagaataaataataggaaagtgattacaaatattacctccagggttcaatagaaatcctcaatttagggtgaggagacttttttttggttttggggtttttccttgattgattttgttttcatagtgggaatcaggattgtgctttattgagcctgcagttacattgaattgtaggtgtttcgtgtgctgctaaggtatgcttatttgagtttatcaagacttttttttttctggaagacactgctgcttttaccatcacattggagcc

SEQ ID NO: 18 - protein, 419 aa, Homo sapiens
MVFSAVLTAFHTGTSNTTFVVYENTYMNITLPPPFQHPDLSPLLRYSFETMAPTGLSSLTVNSTAVPTTPAAFKSLNLPLQITLSAIMIFILFVSFLGNLVVCLMVYQKAAMRSAINILLASLAFADMLLAVLNMPFALVTILTTRWIFGKFFCRVSAMFFWLFVIEGVAILLIISIDRFLIIVQRQDKLNPYRAKVLIAVSWATSFCVAFPLAVGNPDLQIPSRAPQCVFGYTTNPGYQAYVILISLISFFIPFLVILYSFMGILNTLRHNALRIHSYPEGICLSQASKLGLMSLQRPFQMSIDMGFKTRAFTTILILFAVFIVCWAPFTTYSLVATFSKHFYYQHNFFEISTWLLWLCYLKSALNPLIYYWRIKKFHDACLDMMPKSFKFLPQLPGHTKRRIRPSAVYVCGEHRTVV

SEQ ID NO: 19 - DNA, 3689 bp, Homo sapiens
ggaagactacacattttaggtatgtgattagaaaacatacttgtcagaattgtctggctggattaatttgctaatttgaccttcttcatcatttgatgtgatgccagatactaatagcacaatcaatttatcactaagcactcgtgttactttagcattttttatgtccttagtagcttttgctataatgctaggaaatgctttggtcattttagcttttgtggtggacaaaaaccttagacatcgaagtagttatttttttcttaacttggccatctctgacttctttgtgggtgtgatctccattcctttgtacatccctcacacgctgttcgaatgggattttggaaaggaaatctgtgtattttggctcactactgactatctgttatgtacagcatctgtatataacattgtcctcatcagctatgatcgatacctgtcagtctcaaatgctgtgtcttatagaactcaacatactggggtcttgaagattgttactctgatggtggccgtttgggtgctggccttcttagtgaatgggccaatgattctagtttcagagtcttggaaggatgaaggtagtgaatgtgaacctggatttttttcggaatggtacatccttgccatcacatcattcttggaattcgtgatcccagtcatcttagtcgcttatttcaacatgaatatttattggagcctgtggaagcgtgatcatctcagtaggtgccaaagccatcctggactgactgctgtctcttccaacatctgtggacactcattcagaggtagactatcttcaaggagatctctttctgcatcgacagaagttcctgcatcctttcattcagagagacagaggagaaagagtagtctcatgttttcctcaagaaccaagatgaatagcaatacaattgcttccaaaatgggttccttctcccaatcagattctgtagctcttcaccaaagggaacatgttgaactgcttagagccaggagattagccaagtcactggccattctcttaggggtttttgctgtttgctgggctccatattctctgttcacaattgtcctttcattttattcctcagcaacaggtcctaaatcagtttggtatagaattgcattttggcttcagtggttcaattcctttgtcaatcctcttttgtatccattgtgtcacaagcgctttcaaaaggctttcttgaaaatattttgtataaaaaagcaacctctaccatcacaacacagtcggtcagtatcttcttaaagacaattttctcacctctgtaaattttagtctcaatctcacctaaatgaatcaggtctgccctttatcttgcccttttcattctaccaacagatctgcactttgaagtcaatggtaaattactccagtgaataatagcagtataatatgacttgataatatttttgtaaacttgtagtcataatagtactatattcttcttagtcctcacctcttccttgtcttttagatcttaatttcatgctgattacaaaaatccagttttgttttctttctatgttccatgcataatacagtcttaagtgaatttctcttttttaattttatcgtaatagaaacttatccagtttgaaaatcattccctaaagcatgcaataggaaaaagaacctcctggctgggactgcccaactctgttctgatcagtgggtgggtgaggtagggtttgagttggcaagagcagggaacgggcatgtgcccaggtgagctcctgtgtgtgtccagattttatattcctaatcccagtaaggaagaaagcgtagtgtgggagaggagagagctgatgactgcagttctcaaaggtcctcagtgaagttattttggaggccctggtggtcacaggatcagaaggcaagggataggcagtggtcaccaatggttgaaagtatggcttgtcccatttcttcctgttctctttttctagcttccacatcagcttccttttttgagaacatatagaagaagaaggctaagagatggtgaagagactgcatgattaaactagatagacctggtatacagtcactgaactagtagatgtcaataattattatttttaaaaatttttatttgttggccgggcatggtggctcacgcctgaaatcccagcactttgggaggccaaggtgggcggatcatgaggtcaggagatcgagaccatcctggccaacatggtgaaaccccatctgtactaaaatacaaacaagtagctggttgtggcgccgcatgcctgtagtcccagctactcgggaggctgaggcaggggaattgcttgaacccgggaggcggagttttgccagcctggcaacagagcaagactctgtctaaaaagaaaaaaaaatttttttgtttgagacagcatcttgctctgtctcccaggctggagcgtagtaatgcaatcatagctcactgcagcctggaactccttggctcaagcaatcctgctgccttggcctcccaagtatgtgggactacaggtactcgccaccacacctggataattaaaaaattatttctgtagagatgaagtctcactgtgttgcccagcctgggtgtcaataattattttttaaaaaaaatttttaaaaaggttttttgagacagattcttgctctgtcacccaggctggagtgcagtagcatgatcagggatcactgcaacctctgcctcctgggttcaagcgattcttgtgcctaagccacctgagcagctgggattgcaggtgcatgccaccatgcctggctaattttggtatttttagtagagatgaggttttgccattttggtcaggctggaattttttttttttttaattttgataagacagggtattgccgtgttggccagactggtctcaaactcctgggctgaaacaatcctcccgccttggcctcccaaagtgctgggattataggcacaagacaccacaataattattgcctgtatgtcaattattattttaaaatattgttgtatttacttaatgtctttaatgcatttgcccaatattttacattgttactgctcagaggtattcctttattatgtggttagcataggttatactttgctgacgattcacattttattagtttggttatgttttgtccttttaaaacattttcttttgagatgggggtcttgctctgttgcccacgcaggagtgcagtggcatgctctcagctcactgcagccctgactgcctaggctccagcaatcttcttacgtcagcctccagagtagctgggaccgcaggcacttgccaccacgccccactaaaaattttttaaattgttgcctttcttgaagtgttctctgcctgtctttgtcacaaaatttcatttttctcatagttaatttcatctctccggtaagattttattggtgtttcttttataactttgcagttcttacaccgtttggtgattttcatgtttcttagaaactttaaacctttaacttcaaacattaaaatacaagtcttttaagtacatgagtgcttagaaatgtacataatgtttatatacacttatgccttacattaaagtccaatatgagaaatacatgtttaacattcaataataattttaaaaatttgagaaataaactctcataaatgcaaaaaaaaaaaaaaaaaaa

SEQ ID NO: 20 - protein, 390 aa, Homo sapiens
MPDTNSTINLSLSTRVTLAFFMSLVAFAIMLGNALVILAFVVDKNLRHRSSYFFLNLAISDFFVGVISIPLYIPHTLFEWDFGKEICVFWLTTDYLLCTASVYNIVLISYDRYLSVSNAVSYRTQHTGVLKIVTLMVAVWVLAFLVNGPMILVSESWKDEGSECEPGFFSEWYILAITSFLEFVIPVILVAYFNMNIYWSLWKRDHLSRCQSHPGLTAVSSNICGHSFRGRLSSRRSLSASTEVPASFHSERQRRKSSLMFSSRTKMNSNTIASKMGSFSQSDSVALHQREHVELLRARRLAKSLAILLGVFAVCWAPYSLFTIVLSFYSSATGPKSVWYRIAFWLQWFNSFVNPLLYPLCHKRFQKAFLKIFCIKKQPLPSQHSRSVSS

SEQ ID NO: 21 - DNA, 921 bp, Homo sapiens
atgtattcatttatggcaggatccatattcatcacaatatttggcaatcttgccatgataatttccatttcctacttcaagcagcttcacacaccaaccaacttcctcatcctctccatggccatcactgatttcctcctgggattcaccatcatgccatatagtatgatcagatcggtggagaactgctggtattttgggcttacattttgcaagatttattatagttttgacctgatgcttagcataacatccatttttcatctttgctcagtggccattgatagattttatgctatatgttacccattactttattccaccaaaataactattccagtcattaaaagattgctacttctatgttggtcggtccctggagcatttgccttcggggcggtcttctcagaggcctatgcagatggaatagagggctatgacatcttggttgcttgttccagttcctgcccagtgatgttcaacaagctatgggggaccaccttgtttatggcaggtttcttcactcctgggtctatgatggtggggatttacggcaaaatttttgcagtatccagaaaacatgctcatgccatcaataacttgcgagaaaatcaaaataatcaagtgaagaaagacaaaaaagctgccaaaactttaggaatagtgataggagttttcttattatgttggtttccttgtttcttcacaattttattggatccctttttgaacttctctactcctgtagttttgtttgatgccttgacatggtttggctattttaactccacatgtaatccgttaatatatggtttcttctatccctggtttcgcagagcactgaagtacattttgctaggtaaaattttcagctcatgtttccataatactattttgtgtatgcaaaaagaaagtgagtag

SEQ ID NO: 22 - protein, 306 aa, Homo sapiens
MYSFMAGSIFITIFGNLAMIISISYFKQLHTPTNFLILSMAITDFLLGFTIMPYSMIRSVENCWYFGLTFCKIYYSFDLMLSITSIFHLCSVAIDRFYAICYPLLYSTKITIPVIKRLLLLCWSVPGAFAFGAVFSEAYADGIEGYDILVACSSSCPVMFNKLWGTTLFMAGFFTPGSMMVGIYGKIFAVSRKHAHAINNLRENQNNQVKKDKKAAKTLGIVIGVFLLCWFPCFFTILLDPFLNFSTPVVLFDALTWFGYFNSTCNPLIYGFFYPWFRRALKYILLGKIFSSCFHNTILCMQKESE

SEQ ID NO: 23 - DNA, 1849 bp, Homo sapiens
acttagaggcgcctggtcgggaagggcctggtcagctgcgtccggcggaggcagctgctgacccagctgtggactgtgccgggggcgggggacggaggggcaggagccctgggctccccgtggcgggggctgtatcatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttg

SEQ ID NO: 24 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 25 - DNA, 2306 bp, Homo sapiens
gagcttgagaattgctcctgccctgggaagaggctcagcacagaaagaggaaggacagcacagctgacagccgtgctcagagagtttctggatcctaggcttatctccacagaggagaacacacaagcagcagagaccatgggaaccctctcagcccctccctgcacacagcgcatcaaatggaaggggctcctgctcacagcatcacttttaaacttctggaacctgcccaccactgcccaagtcacgattgaagccgagccaaccaaagtttccgaggggaaggatgttcttctacttgtccacaatttgccccagaatcttaccggctacatctggtacaaagggcaaatgagggacctctaccattacattacatcatatgtagtagacggtgaaataattatatatgggcctgcatatagtggacgagaaacagcatattccaatgcatccctgctgatccagaatgtcacccgggaggacgcaggatcctacaccttacacatcataaagggagatgatgggactagaggagtaactggacgtttcaccttcaccttacacctggagactcctaagccctccatctccagcagcaacttaaatcccagggagaccatggaggctgtgagcttaacctgtgaccctgagactccagacgcaagctacctgtggtggatgaatggtcagagcctccctatgactcacagcttgaagctgtccgaaaccaacaggaccctctttctattgggtgtcacaaagtatactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccgttcaggagaagtcctctacttgtcctgttctgcggactctaacccaccggcacagtattcttggacaattaatgaaaagtttcagctaccaggacaaaagctctttatccgccatattactacaaagcatagcgggctctatgtttgctctgttcgtaactcagccactggcaaggaaagctccaaatccatgacagtcgaagtctctggtaagtggatcccagcatcgttggcaatagggttttaggtggagtctatctggcattcagagaagagtcaggaaaacaattgtattcccagcctgtgtcccatgggcacaagcaaatcccaaattctcctcctgaaccctccaaatttgtctaagaacttcgaaaactttaacaaacaggctgatatcttcataatattcccagcctagaccaagcaggaagaacattgatttcattgaaataattgataataatgaagataatgtttttatgatttttatttgaaaatttgctgattctttaaatggtttgttttctacattgatggaatttttctcttttaatctatctacagcttatagcagttcaataaactatacttctgggaaccgtaattgaaacatttacttttgctttctacctgactgccccagaattgggcaactattcatgagaattgatatgtttatggtaatacacatatttgcacaagtacagtaacaatctgctttctttgtaacatgacacatttgaaatcattggttatattaccaatgctttgattcggatgttatattaaaaacatagatagaatgaaccaatatgaactgcaggcaaagtctgaagtcagccttggtttggcttcctattctcaagaggtttgtgaagatttaatctcagattccttataaaaacttagagaaaagaaaattttagaagacagcctacatggtccattgctactcttgctgcacttatgtaaacaatcagaccacatttgaagaaactccacctattttgcaaacaaacttattctactgaaattatcattggtaaaagtagagatgcccatagagggaaaaattatgtggaaaataaaaactgtagtatacctaaaaaaaaaaaaaaaaaaaaaaaaaaaaaa

SEQ ID NO: 26 - protein, 426 aa, Homo sapiens
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSGKWIPASLAIGF

SEQ ID NO: 27 - DNA, 1856 bp, Homo sapiens
gcacagctgagagccatgctcaggaagtttctggatcctaggctcagctccacagaggagaacacgcaggcagcagagaccatggggcccctctcagcccctccctgcacacagcgcatcacctggaaggggctcctgctcacagcatcacttttaaacttctggaacccgcctaccactgcccaagtcacgattgaagccgagccaaccaaagtttccaaggggaaggacgttcttctacttgtccacaatttgccccagaatcttgctggctacatctggtacaaagggcaaatgaaggacctctaccattacattacatcatacgtagtagatggtcaaataattatatatgggcctgcatacagtggacgagaaacagtatattccaatgcatccctgctgatccagaatgtcacccgggaggacgcaggatcctacaccttacacatcgtaaagcgaggtgatgggactagaggagaaactggacatttcaccttcaccttatacctggagactcccaagccctccatctccagcagcaacttataccccagggaggacatggaggctgtgagcttaacctgtgatcctgagactccggacgcaagctacctgtggtggatgaatggtcagagcctccctatgactcacagcttgcagttgtccaaaaacaaaaggaccctctttctatttggtgtcacaaagtacactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttagccttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacaggaccgatatggtggcatccgcagttacccagtcaccctgaatgtcctctatggtccagacctccccagaatttacccttcattcacctattaccattcaggagaaaacctctacttgtcctgcttcgcggactctaacccaccagcagaatattcttggacaattaatgggaagtttcagctatcaggacaaaagctctttatcccccagattactacaaagcatagcgggctctatgcttgctctgttcgtaactcagccactggcatggaaagctccaaatccatgacagtcaaagtctctgctccttcaggaacaggacatcttcctggccttaatccattatagcagccgtgatgtcatttctgtatttcaggaagactggcagacagttgctttcattcttcctcaaagtatttaccatcagctacagtccaaaattgctttttgttcaaggagatttatgaaaagactctgacaaggactcttgaatacaagttcctgataacttcaagatcataccactggactaagaactttcaaaattttaatgaacaggctgatacttcatgaaattcaagacaaagaaaaaaacccaattttattggactaaatagtcaaaacaatgttttcataattttctatttgaaaatgtgctgattctttgaatgttttattctccagatttatgcactttttttcttcagcaattggtaaagtatacttttgtaaacaaaaattgaaacatttgcttttgctccctaagtgccccagaattgggaaactattcaggagtattcatatgtttatggtaataaagttatctgcacaagttc

SEQ ID NO: 28 - protein, 428 aa, Homo sapiens
MGPLSAPPCTQRITWKGLLLTASLLNFWNPPTTAQVTIEAEPTKVSKGKDVLLLVHNLPQNLAGYIWYKGQMKDLYHYITSYVVDGQIIIYGPAYSGRETVYSNASLLIQNVTREDAGSYTLHIVKRGDGTRGETGHFTFTLYLETPKPSISSSNLYPREDMEAVSLTCDPETPDASYLWWMNGQSLPMTHSLQLSKNKRTLFLFGVTKYTAGPYECEIRNPVSASRSDPVTLNLLPKLPKPYITINNLNPRENKDVLAFTCEPKSENYTYIWWLNGQSLPVSPRVKRPIENRILILPSVTRNETGPYQCEIQDRYGGIRSYPVTLNVLYGPDLPRIYPSFTYYHSGENLYLSCFADSNPPAEYSWTINGKFQLSGQKLFIPQITTKHSGLYACSVRNSATGMESSKSMTVKVSAPSGTGHLPGLNPL

SEQ ID NO: 29 - DNA, 1409 bp, Homo sapiens
gggcgggcctaggctcatctccacaggggagaacacacagacagcagagaccatgggacccctctcagcccctccctgcactcagcacatcacctggaaggggctcctgctcacagcatcacttttaaacttctggaacctgcccaccactgcccaagtaataattgaagccaagccacccaaagtttccgaggggaaggatgttcttctacttgtccacaatttgccccagaatcttactggctacatctggtacaaagggcaaatgacggacctctaccattacattacatcatatgtagtacacggtcaaattatatatgggcctgcctacagtggacgagaaacagtatattccaatgcatccctgctgatccagaatgtcacacaggaggatgcaggatcctacaccttacacatcataaagcgaggcgatgggactggaggagtaactggatatttcactgtcaccttatactcggagactcccaagccctccatctccagcagcaacttaaaccccagggaggtcatggaggctgtgcgcttaatctgtgatcctgagactccggatgcaagctacctgtggttgctgaatggtcagaacctccctatgactcacaggttgcagctgtccaaaaccaacaggaccctctatctatttggtgtcacaaagtatattgcaggaccctatgaatgtgaaatacggaacccagtgagtgccagccgcagtgacccagtcaccctgaatctcctcccgaagctgcccatgccttacatcaccatcaacaacttaaaccccagggagaagaaggatgtgttagccttcacctgtgaacctaagagtcggaactacacctacatttggtggctaaatggtcagagcctcccggtcagtccgagggtaaagcgacccattgaaaacaggatactcattctacccagtgtcacgagaaatgaaacaggaccctatcaatgtgaaatacgggaccgatatggtggcatccgcagtaacccagtcaccctgaatgtcctctatggtccagacctccccagaatttacccttcattcacctattaccgttcaggagaaaacctcgacttgtcctgctttgcggactctaacccaccggcagagtattcttggacaattaatgggaagtttcagctatcaggacaaaagctctttatcccccaaattactacaaatcatagcgggctctatgcttgctctgttcgtaactcagccactggcaaggaaatctccaaatccatgatagtcaaagtctctgagacagcatctccccaggttacctatgctggtccaaacacctggtttcaagaaatccttctgctgtgacctcccaaagtgctaggattaaaacatgacccaccatgaaacccgccca

SEQ ID NO: 30 - protein, 435 aa, Homo sapiens
MGPLSAPPCTQHITWKGLLLTASLLNFWNLPTTAQVIIEAKPPKVSEGKDVLLLVHNLPQNLTGYIWYKGQMTDLYHYITSYVVHGQIIYGPAYSGRETVYSNASLLIQNVTQEDAGSYTLHIIKRGDGTGGVTGYFTVTLYSETPKPSISSSNLNPREVMEAVRLICDPETPDASYLWLLNGQNLPMTHRLQLSKTNRTLYLFGVTKYIAGPYECEIRNPVSASRSDPVTLNLLPKLPMPYITINNLNPREKKDVLAFTCEPKSRNYTYIWWLNGQSLPVSPRVKRPIENRILILPSVTRNETGPYQCEIRDRYGGIRSNPVTLNVLYGPDLPRIYPSFTYYRSGENLDLSCFADSNPPAEYSWTINGKFQLSGQKLFIPQITTNHSGLYACSVRNSATGKEISKSMIVKVSETASPQVTYAGPNTWFQEILLL

SEQ ID NO: 31 - DNA, 1731 bp, Homo sapiens
agaaggaggaaggacagcacagctgacagccgtgctcagacagcttctggatcccaggctcatctccacagaggagaacacacaggcagcagagaccatggggcccctcccagccccttcctgcacacagcgcatcacctggaaggggctcctgctcacagcatcacttttaaacttctggaacccgcccaccactgccgaagtcacgattgaagcccagccacccaaagtttctgaggggaaggatgttcttctacttgtccacaatttgccccagaatcttcctggctacttctggtacaaaggggaaatgacggacctctaccattacattatatcgtatatagttgatggtaaaataattatatatgggcctgcatacagtggaagagaaacagtatattccaacgcatccctgctgatccagaatgtcacccggaaggatgcaggaacctacaccttacacatcataaagcgaggtgatgagactagagaagaaattcgacatttcaccttcaccttatacttggagactcccaagccctacatctccagcagcaacttaaaccccagggaggccatggaggctgtgcgcttaatctgtgatcctgagactctggacgcaagctacctatggtggatgaatggtcagagcctccctgtgactcacaggttgcagctgtccaaaaccaacaggaccctctatctatttggtgtcacaaagtatattgcaggaccctatgaatgtgaaatacggaacccagtgagtgccagtcgcagtgacccagtcaccctgaatctcctcccgaagctgcccatcccctacatcaccatcaacaacttaaaccccagggagaataaggatgtcttagccttcacctgtgaacctaagagtgagaactacacctacatttggtggctaaacggtcagagcctccccgtcagtcccggggtaaagcgacccattgaaaacaggatactcattctacccagtgtcacgagaaatgaaacaggaccctatcaatgtgaaatacaggaccgatatggtggcctccgcagtaacccagtcatcctaaatgtcctctatggtccagacctccccagaatttacccttcattcacctattaccgttcaggagaaaacctcgacttgtcctgcttcacggaatctaacccaccggcagagtatttttggacaattaatgggaagtttcagcaatcaggacaaaagctctttatcccccaaattactagaaatcatagcgggctctatgcttgctctgttcataactcagccactggcaaggaaatctccaaatccatgacagtcaaagtctctggtccctgccatggagacctgacagagtctcagtcatgactgcaacaactgagacactgagaaaaagaacaggctgataccttcatgaaattcaagacaaagaagaaaaaaactcaatgttattggactaaataatcaaaaggataatgttttcataattttttattggaaaatgtgctgattctttgaatgttttattctccagatttatgaactttttttcttcagcaattggtaaagtatacttttataaacaaaaattgaaatatttgcttttgctgtctatctgaatgccccagaattgtgaaactattcatgagtattcataggtttatggtaataaagttatttgcacatgttccaaaaaaaaaaaaaaaaaaaaaaaaaaaaaa

SEQ ID NO: 32 - protein, 426 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIQDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 33 - DNA, 13109 bp, Homo sapiens
ggggaagcagtggccgtgtgagcgtgaggagctgccgccaccgcctgctcctcgtcctcctcgtcctccggggccccagcgtcgtgggccgcgcacggccctggaagagacgtcgcctcgccttcatccgcctctctcaccgcgccgctccctcgtcctgccctgcgggctcaggcggaacccggaacggccgtcctcttcccccgccctccgccgccgcctcctcctcctccttctcggcttcctcctcagccccgggccggagcggggtgtcggcggcggccggttcgggcggcggcgcttggccatgtcgtgtcggggaaggtaatgagccgcagagccccggggtctcggctgagcagcggcggcaccaactattcgcggagctggaatgactggcaacccagaactgatagtgcatcagctgacccaggtaatttaaaatattcttcatccagagatagaggtggttcttcctcttacggactgcaaccttcaaattcagctgtggtgtctcggcaaaggcacgatgataccagagtccacgctgacatacagaatgacgaaaagggtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttaaatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttcttcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaagaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagtgctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcacgctggtgagcttcggcagtatctggagcacggcttggagacaacacccactgcaccatggagaggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaagcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcgacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggcgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 34 - protein, 3657 aa, Homo sapiens
MSRRAPGSRLSSGGTNYSRSWNDWQPRTDSASADPGNLKYSSSRDRGGSSSYGLQPSNSAVVSRQRHDDTRVHADIQNDEKGGYSVNGGSGENTYGRKSLGQELRVNNVTSPEFTSVQHGSRALATKDMRKSQERSMSYSDESRLSNLLRRITREDDRDRRLATVKQLKEFIQQPENKLVLVKQLDNILAAVHDVLNESSKLLQELRQEGACCLGLLCASLSYEAEKIFKWIFSKFSSSAKDEVKLLYLCATYKALETVGEKKAFSSVMQLVMTSLQSILENVDTPELLCKCVKCILLVARCYPHIFSTNFRDTVDILVGWHIDHTQKPSLTQQVSGWLQSLEPFWVADLAFSTTLLGQFLEDMEAYAEDLSHVASGESVDEDVPPPSVSLPKLAALLRVFSTVVRSIGERFSPIRGPPITEAYVTDVLYRVMRCVTAANQVFFSEAVLTAANECVGVLLGSLDPSMTIHCDMVITYGLDQLENCQTCGTDYIISVLNLLTLIVEQINTKLPSSFVEKLFIPSSKLLFLRYHKEKEVVAVAHAVYQAVLSLKNIPVLETAYKLILGEMTCALNNLLHSLQLPEACSEIKHEAFKNHVFNVDNAKFVVKFDLSALTTIGNAKNSLIGMWALSPTVFALLSKNLMIVHSDLAVHFPAIQYAVLYTLYSHCTRHDHFISSSLSSSSPSLFDGAVISTVTTATKKHFSIILNLLGILLKKDNLNQDTRKLLMTWALEAAVLMKKSETYAPLFSLPSFHKFCKGLLANTLVEDVNICLQACSSLHALSSSLPDDLLQRCVDVCRVQLVHSGTRIRQAFGKLLKSIPLDVVLSNNNHTEIQEISLALRSHMSKAPSNTFHPQDFSDVISFILYGNSHRTGKDNWLERLFYSCQRLDKRDQSTIPRNLLKTDAVLWQWAIWEAAQFTVLSKLRTPLGRAQDTFQTIEGIIRSLAAHTLNPDQDVSQWTTADNDEGHGNNQLRLVLLLQYLENLEKLMYNAYEGCANALTSPPKVIRTFFYTNRQTCQDWLTRIRLSIMRVGLLAGQPAVTVRHGFDLLTEMKTTSLSQGNELEVTIMMVVEALCELHCPEAIQGIAVWSSSIVGKNLLWINSVAQQAEGRFEKASVEYQEHLCAMTGVDCCISSFDKSVLTLANAGRNSASPKHSLNGESRKTVLSKPTDSSPEVINYLGNKACECYISIADWAAVQEWQNAIHDLKKSTSSTSLNLKADFNYIKSLSSFESGKFVECTEQLELLPGENINLLAGGSKEKIDMKKLLPNMLSPDPRELQKSIEVQLLRSSVCLATALNPIEQDQKWQSITENVVKYLKQTSRIAIGPLRLSTLTVSQSLPVLSTLQLYCSSALENTVSNRLSTEDCLIPLFSEALRSCKQHDVRPWMQALRYTMYQNQLLEKIKEQTVPIRSHLMELGLTAAKFARKRGNVSLATRLLAQCSEVQLGKTTTAQDLVQHFKKLSTQGQVDEKWGPELDIEKTKLLYTAGQSTHAMEMLSSCAISFCKSVKAEYAVAKSILTLAKWIQAEWKEISGQLKQVYRAQHQQNFTGLSTLSKNILTLIELPSVNTMEEEYPRIESESTVHIGVGEPDFILGQLYHLSSVQAPEVAKSWAALASWAYRWGRKVVDNASQGEGVRLLPREKSEVQNLLPDTITEEEKERIYGILGQAVCRPAGIQDEDITLQITESEDNEEDDMVDVIWRQLISSCPWLSELDESATEGVIKVWRKVVDRIFSLYKLSCSAYFTFLKLNAGQIPLDEDDPRLHLSHRVEQSTDDMIVMATLRLLRLLVKHAGELRQYLEHGLETTPTAPWRGIIPQLFSRLNHPEVYVRQSICNLLCRVAQDSPHLILYPAIVGTISLSSESQASGNKFSTAIPTLLGNIQGEELLVSECEGGSPPASQDSNKDEPKSGLNEDQAMMQDCYSKIVDKLSSANPTMVLQVQMLVAELRRVTVLWDELWLGVLLQQHMYVLRRIQQLEDEVKRVQNNNTLRKEEKIAIMREKHTALMKPIVFALEHVRSITAAPAETPHEKWFQDNYGDAIENALEKLKTPLNPAKPGSSWIPFKEIMLSLQQRAQKRASYILRLEEISPWLAAMTNTEIALPGEVSARDTVTIHSVGGTITILPTKTKPKKLLFLGSDGKSYPYLFKGLEDLHLDERIMQFLSIVNTMFATINRQETPRFHARHYSVTPLGTRSGLIQWVDGATPLFGLYKRWQQREAALQAQKAQDSYQTPQNPGIVPRPSELYYSKIGPALKTVGLSLDVSRRDWPLHVMKAVLEELMEATPPNLLAKELWSSCTTPDEWWRVTQSYARSTAVMSMVGYIIGLGDRHLDNVLIDMTTGEVVHIDYNVCFEKGKSLRVPEKVPFRMTQNIETALGVTGVEGVFRLSCEQVLHIMRRGRETLLTLLEAFVYDPLVDWTAGGEAGFAGAVYGGGGQQAESKQSKREMEREITRSLFSSRVAEIKVNWFKNRDEMLVVLPKLDGSLDEYLSLQEQLTDVEKLQGKLLEEIEFLEGAEGVDHPSHTLQHRYSEHTQLQTQQRAVQEAIQVKLNEFEQWITHYQAAFNNLEATQLASLLQEISTQMDLGPPSYVPATAFLQNAGQAHLISQCEQLEGEVGALLQQRRSVLRGCLEQLHHYATVALQYPKAIFQKHRIEQWKTWMEELICNTTVERCQELYRKYEMQYAPQPPPTVCQFITATEMTLQRYAADINSRLIRQVERLKQEAVTVPVCEDQLKEIERCIKVFLHENGEEGSLSLASVIISALCTLTRRNLMMEGAASSAGEQLVDLTSRDGAWFLEELCSMSGNVTCLVQLLKQCHLVPQDLDIPNPMEASETVHLANGVYTSLQELNSNFRQIIFPEALRCLMKGEYTLESMLHELDGLIEQTTDGVPLQTLVESLQAYLRNAAMGLEEETHAHYIDVARLLHAQYGELIQPRNGSVDETPKMSAGQMLLVAFDGMFAQVETAFSLLVEKLNKMEIPIAWRKIDIIREARSTQVNFFDDDNHRQVLEEIFFLKRLQTIKEFFRLCGTFSKTLSGSSSLEDQNTVNGPVQIVNVKTLFRNSCFSEDQMAKPIKAFTADFVRQLLIGLPNQALGLTLCSFISALGVDIIAQVEAKDFGAESKVSVDDLCKKAVEHNIQIGKFSQLVMNRATVLASSYDTAWKKHDLVRRLETSISSCKTSLQRVQLHIAMFQWQHEDLLINRPQAMSVTPPPRSAILTSMKKKLHTLSQIETSIATVQEKLAALESSIEQRLKWAGGANPALAPVLQDFEATIAERRNLVLKESQRASQVTFLCSNIIHFESLRTRTAEALNLDAALFELIKRCQQMCSFASQFNSSVSELELRLLQRVDTGLEHPIGSSEWLLSAHKQLTQDMSTQRAIQTEKEQQIETVCETIQNLVDNIKTVLTGHNRQLGDVKHLLKAMAKDEEAALADGEDVPYENSVRQFLGEYKSWQDNIQTVLFTLVQAMGQVRSQEHVEMLQEITPTLKELKTQSQSIYNNLVSFASPLVTDATNECSSPTSSATYQPSFAAAVRSNTGQKTQPDVMSQNARKLIQKNLATSADTPPSTVPGTGKSVACSPKKAVRDPKTGKAVQERNSYAVSVWKRVKAKLEGRDVDPNRRMSVAEQVDYVIKEATNLDNLAQLYEGWTAWV

SEQ ID NO: 35 - DNA, 3130 bp, Homo sapiens
gaattcccaatacttgttgcaataattgcccacgatagctgctcaaacaagagagttggaattcatctgtaaaaatcactacatgtaacgtaggagacaagaaaaatattaatgacagaagatctgcgaacatgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgttcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatac

SEQ ID NO: 36 - protein, 673 aa, Homo sapiens
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 37 - DNA, 2395 bp, Homo sapiens
gccgccgtcggcgcgctgggtgcgggaagggggctctggatttcggtccctcccctttttcctctgagtctcggaacgctccagctctcagaccctcttcctcccaggtaaaggccgggagaggagggcgcatctcttttccaggcaccccaccatgggcaatgcctccaatgactcccagtctgaggactgcgagacgcgacagtggcttcccccaggcgaaagcccagccatcagctccgtcatgttctcggccggggtgctggggaacctcatagcactggcgctgctggcgcgccgctggcggggggacgtggggtgcagcgccggccgcaggagctccctctccttgttccacgtgctggtgaccgagctggtgttcaccgacctgctcgggacctgcctcatcagcccagtggtactggcttcgtacgcgcggaaccagaccctggtggcactggcgcccgagagccgcgcgtgcacctacttcgctttcgccatgaccttcttcagcctggccacgatgctcatgctcttcgccatggccctggagcgctacctctcgatcgggcacccctacttctaccagcgccgcgtctcgcgctccgggggcctggccgtgctgcctgtcatctatgcagtctccctgctcttctgctcgctgccgctgctggactatgggcagtacgtccagtactgccccgggacctggtgcttcatccggcacgggcggaccgcttacctgcagctgtacgccaccctgctgctgcttctcattgtctcggtgctcgcctgcaacttcagtgtcattctcaacctcatccgcatgcaccgccgaagccggagaagccgctgcggaccttccctgggcagtggccggggcggccccggggcccgcaggagaggggaaagggtgtccatggcggaggagacggaccacctcattctcctggctatcatgaccatcaccttcgccgtctgctccttgcctttcacgatttttgcatatatgaatgaaacctcttcccgaaaggaaaaatgggacctccaagctcttaggtttttatcaattaattcaataattgacccttgggtctttgccatccttaggcctcctgttctgagactaatgcgttcagtcctctgttgtcggatttcattaagaacacaagatgcaacacaaacttcctgttctacacagtcagatgccagtaaacaggctgacctttgaggtcagtagtttaaaagttcttagttatatagcatctggaagatcattttgaaattgttccttggagaaatgaaaacagtgtgtaaacaaaatgaagctgccctaataaaaaggagtatacaaacatttaagctgtggtcaaggctacagatgtgctgacaaggcacttcatgtaaagtgtcagaaggagctacaaaacctaccctcagtgagcatggtacttggcctttggaggaacaatcggctgcattgaagatccagctgcctattgatttaagctttcctgttgaatgacaaagtatgtggttttgtaatttgtttgaaaccccaaacagtgactgtactttctattttaatcttgctactaccgttatacacatatagtgtacagccagaccagattaaacttcatatgtaatctctaggaagtcaatatgtggaagcaaccaagcctgctgtcttgtgatcacttagcgaaccctttatttgaacaatgaagttgaaaatcataggcaccttttactgtgatgtttgtgtatgtgggagtactctcatcactacagtattactcttacaagagtggactcagtgggttaacatcagttttgtttactcatcctccaggaactgcaggtcaagttgtcaggttatttattttataatgtccatatgctaatagtgatcaagaagactttaggaatggttctctcaacaagaaataatagaaatgtctcaaggcagttaattctcattaatactcttattatcctatttctgggggaggatgtacgtggccatgtatgaagccaaatattaggcttaaaaactgaaaaatctggttcattcttcagatatactggaacccttttaaagttgatattggggccatgagtaaaatagattttataagatgactgtgttgtaccaaaattcatctgtctatattttatttagggaacatggtttgactcatcttatatgggaaaccatgtagcagtgagtcatatcttaatatatttctaaatgtttggcatgtaaatgtaaactcagcatcaaaatatttcagtgaatttgcactgtttaatcatagttactgtgtaaactcatctgaaatgttacaaaaataaactataaaacaaaaatttgaaaaaaaaaaaaaaaaaa

SEQ ID NO: 38 - protein, 358 aa, Homo sapiens
MGNASNDSQSEDCETRQWLPPGESPAISSVMFSAGVLGNLIALALLARRWRGDVGCSAGRRSSLSLFHVLVTELVFTDLLGTCLISPVVLASYARNQTLVALAPESRACTYFAFAMTFFSLATMLMLFAMALERYLSIGHPYFYQRRVSRSGGLAVLPVIYAVSLLFCSLPLLDYGQYVQYCPGTWCFIRHGRTAYLQLYATLLLLLIVSVLACNFSVILNLIRMHRRSRRSRCGPSLGSGRGGPGARRRGERVSMAEETDHLILLAIMTITFAVCSLPFTIFAYMNETSSRKEKWDLQALRFLSINSIIDPWVFAILRPPVLRLMRSVLCCRISLRTQDATQTSCSTQSDASKQADL

SEQ ID NO: 39 - DNA, 2745 bp, Homo sapiens
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggccaggcggggtgctcacgcctgtaatcctagcacttttgggaggcagagatgggaggatacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 40 - protein, 391 aa, Homo sapiens
MPPSGLRLLPLLLPLLWLLVLTPGPPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

(19) United States
(12) Patent Application Publication
(10) Pub. No.: US 2009/0318357 A1
(43) Pub. Date: Dec. 24, 2009

(54) Molecules Preferentially Associated with Effector T Cells or Regulatory T Cells and Methods of Their Use

(75) Inventors: Patricia Rao, Acton, MA (US); Grazyna Szymanska, Dedham, MA (US)

Correspondence Address: FISH & RICHARDSON P.C., PO BOX 1022, MINNEAPOLIS, MN, 55440-1022, US

(73) Assignee: Tolerx, Inc., Cambridge, MA (US)

(21) Appl. No.: 12/483,826

(22) Filed: Jun. 12, 2009

Related U.S. Application Data
(63) Continuation of application No. 10/684,206, filed on Oct. 9, 2003, now abandoned.
(60) Provisional application No. 60/417,102, filed on Oct. 9, 2002.
(60) Provisional application No. 60/417,103, filed on Oct. 9, 2002.
(60) Provisional application No. 60/417,243, filed on Oct. 9, 2002.
(60) Provisional application No. 60/419,575, filed on Oct. 18, 2002.
(60) Provisional application No. 60/424,777, filed on Nov. 8, 2002.
(60) Provisional application No. 60/424,881, filed on Nov. 8, 2002.

(51) Int. Cl. A61K 38/16; A61K 31/7088; C07K 2/00; C40B 30/04; G01N 33/53; A61P 37/04

(52) U.S. Cl. 514/12; 514/44 R; 530/300; 506/9; 435/7.2

(57) ABSTRACT

The present invention is based, at least in part, on the finding that certain molecules are preferentially associated with effector T cells or regulatory T cells. Accordingly, immune responses by one or the other subset of cells can be preferentially modulated. The invention pertains, e.g., to methods of modulating (e.g., up- or down-modulating), the balance between the activation of regulatory T cells and effector T cells leading to modulation of immune responses and to compositions useful in modulating those responses. The invention also pertains to methods useful in diagnosing, treating, or preventing conditions that would benefit from modulating effector T cell function relative to regulatory T cell function or from modulating regulatory T cell function relative to effector T cell function in a subject. The subject methods and compositions are especially useful in the diagnosis, treatment or prevention of conditions characterized by a too-vigorous effector T cell response to antigens associated with the condition, in the diagnosis, treatment or prevention of conditions characterized by a weak effector T cell response, in the diagnosis, treatment or prevention of conditions characterized by a too-vigorous regulatory T cell response, or in the diagnosis, treatment, or prevention of conditions characterized by a weak regulatory T cell response.

### RELATED APPLICATIONS

This application is a continuation of U.S. application Ser. No. 10/684,206, filed Oct. 9, 2003, which claims the benefit of U.S. Provisional Application, 60/417,102, filed Oct. 9, 2002, titled “Surface Markers for TH1 and/or TH2 Cells and Reduction of Immune Responses”, U.S. Provisional Application, 60/419,575, filed Oct. 18, 2002, titled “Secreted Proteins of TH1 and/or TH2 Cells and Regulation of Immune Responses”, U.S. Provisional Application, 60/424,777, filed Nov. 8, 2002, titled “Intracellular Proteins of TH1 and/or TH2 Cells and Regulation of Immune Responses”, U.S. Provisional Application, 60/417,103, filed Oct. 9, 2002, titled “Surface Markers of Treg Cells and Method for Increasing Immunogenic Reactions”, U.S. Provisional Application, 60/424,881, filed Nov. 8, 2002, titled “Intracellular Proteins of Treg Cells and Regulation of Immune Responses”, and U.S. Provisional Application, 60/417,243, filed Oct. 9, 2002, titled, “Secreted Proteins of Treg Cells and Regulation of Immune Responses”. The entire contents of each of these applications are incorporated herein by reference.

### SEQUENCE LISTING

This application incorporates herein by reference the sequence listing filed concurrently herewith, i.e., the file “seqlist” (165 KB) created on Jun. 12, 2009.

### BACKGROUND OF THE INVENTION

The immune system provides the human body with a means to recognize and defend itself against microorganisms, viruses, and substances recognized as foreign and potentially harmful. Classical immune responses are initiated when antigen-presenting cells present an antigen to CD4+ T helper (Th) lymphocytes resulting in T cell activation, proliferation, and differentiation of effector T lymphocytes. Following exposure to antigens, such as that which results from infection or the grafting of foreign tissue, naïve T cells differentiate into Th1 and Th2 cells with differing functions. Th1 cells produce interferon gamma (IFN-y) and interleukin 2 (IL-2) (both associated with cell-mediated immune responses). Th1 cells play a role in immune responses commonly involved in the rejection of foreign tissue grafts as well as many autoimmune diseases. Th2 cells produce cytokines such as interleukin-4 (IL-4), and are associated with antibody-mediated immune responses such as those commonly involved in allergies and allergic inflammatory responses such as allergic rhinitis and asthma. Th2 cells may also contribute to the rejection of foreign grafts. In numerous situations, this immune response is desirable, for example, in defending the body against bacterial or viral infection, inhibiting the proliferation of cancerous cells and the like. However, in other situations, such effector T cells are undesirable, e.g., in a graft recipient.

Whether the immune system is activated by or tolerized to an antigen depends upon the balance between T effector cell activation and T regulatory cell activation. T regulatory cells are responsible for the induction and maintenance of immunological tolerance. These cells are T cells which produce low levels of IL-2, IL-4, IL-5, and IL-12. Regulatory T cells produce TNFα, TGFβ, IFN-γ, and IL-10, albeit at lower levels than effector T cells. Although TGFP is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

Until now, undesirable immune responses have been treated with immunosuppressive drugs, which inhibit the entire immune system, i.e., both desired and undesired immune responses. General immunosuppressants must be administered frequently, for prolonged periods of time, and have numerous harmful side effects. Withdrawal of these drugs generally results in relapse of disease. Thus, there is a need for agents that preferentially modulate the effector or regulatory arm of the immune system without modulating the entire immune system.

### SUMMARY OF THE INVENTION

The present invention is based, at least in part, on the finding that certain molecules are preferentially associated with effector T cells or regulatory T cells. Accordingly, immune responses by one or the other subset of cells can be preferentially modulated. The invention pertains, e.g., to methods of modulating (e.g., up- or down-modulating), the balance between the activation of regulatory T cells and effector T cells leading to modulation of immune responses and to compositions useful in modulating those responses. The invention also pertains to methods useful in diagnosing, treating, or preventing conditions that would benefit from modulating effector T cell function relative to regulatory T cell function or from modulating regulatory T cell function relative to effector T cell function in a subject. The subject methods and compositions are especially useful in the diagnosis, treatment or prevention of conditions characterized by a too-vigorous effector T cell response to antigens associated with the condition, in the diagnosis, treatment or prevention of conditions characterized by a weak effector T cell response, in the diagnosis, treatment or prevention of conditions characterized by a too-vigorous regulatory T cell response, or in the diagnosis, treatment, or prevention of conditions characterized by a weak regulatory T cell response.

In one aspect, the invention pertains to a method for treating a subject having a condition that would benefit from modulating the balance of regulatory T cell function relative to effector T cell function in the subject, comprising administering an agent that modulates the expression or activity of a molecule selected from the group consisting of: PTGER2 and TGFβ1 to the subject such that treatment occurs.

In another aspect the invention features a method for treating a subject having a condition that would benefit from modulating the balance of effector T cell function relative to regulatory T cell function in the subject, comprising administering an agent that modulates the expression or activity of a molecule selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase to the subject such that treatment occurs.

In another aspect of the invention, a method is featured for modulating regulatory T cell function relative to effector T cell function in a population of immune cells comprising effector T cells and regulatory T cells contacting the population of cells with an agent that modulates the expression or activity of a molecule selected from the group consisting of: PTGER2 and TGFβ1 in at least a fraction of the immune cells such that treatment occurs.

In yet another aspect, the invention features a method for modulating effector T cell function relative to regulatory T cell function in a population of immune cells comprising effector T cells and regulatory T cells contacting the population of cells with an agent that modulates the expression or activity of a molecule selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase in at least a fraction of the immune cells such that treatment occurs.

In one embodiment, the molecule is a gene and expression of the gene is downmodulated. In another embodiment, the molecule is a polypeptide and activity of the polypeptide is downmodulated. In yet another embodiment, the molecule is a gene and expression of the gene is upmodulated. In another embodiment, the molecule is a polypeptide and activity of the polypeptide is upmodulated.

In one embodiment, effector T cell function is inhibited in said subject relative to regulatory T cell function. In another embodiment, effector T cell function is stimulated in said subject relative to regulatory T cell function.

In one embodiment, the condition is selected from the group consisting of: a transplant, an allergic response, and an autoimmune disorder. In another embodiment, the condition is selected from the group consisting of: a viral infection, a microbial infection, a parasitic infection and a tumor.

In one aspect of the invention, an assay is featured for identifying compounds that modulate at least one regulatory T cell function relative to modulating at least one effector T cell function comprising: contacting an indicator composition comprising a polypeptide selected from the group consisting of: PTGER2 and TGFβ1 with each member of a library of test compounds; determining the ability of the test compound to modulate the activity of the polypeptide, wherein modulation of the activity of the polypeptide indicates that the test compound modulates at least one regulatory T cell function relative to at least one effector T cell function; and selecting from the library a compound of interest.

In another aspect, the invention features an assay for screening compounds that modulate at least one effector T cell function relative to modulating at least one regulatory T cell function comprising: contacting an indicator composition comprising a polypeptide selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase with a test compound; determining the ability of the test compound to modulate the activity of the polypeptide, wherein modulation of the activity of the polypeptide indicates that the test compound modulates at least one effector T cell function relative to at least one regulatory T cell function; and selecting from the library a compound of interest.

In one embodiment, the assay further comprises determining the effect of the compound of interest on at least one T regulatory cell function and at least one T effector cell function in an in vitro or in vivo assay.

In another embodiment, the indicator composition is a cell expressing the polypeptide. In another embodiment, the cell has been engineered to express the polypeptide by introducing into the cell an expression vector encoding the polypeptide. In a further embodiment, the indicator composition is a cell that expresses the polypeptide and a target molecule, and the ability of the test compound to modulate the interaction of the polypeptide with the target molecule is monitored.

In another embodiment, the indicator composition comprises an indicator cell, wherein the indicator cell comprises the polypeptide and a reporter gene sensitive to activity of the polypeptide.

In one embodiment, the indicator composition is a cell free composition.

### BRIEF DESCRIPTION OF THE DRAWINGS

FIG. 1 graphically depicts representative data showing the effect of TGFβ1 on the expression of the transcription factors, GATA3, Tbox21 and FOXP3, in anti-CD3/anti-CD28 stimulated peripheral blood lymphocytes as determined by Real-Time PCR.

FIGS. 2A-2C graphically depicts representative data showing the effect of various concentrations of AH6809 (an antagonist of the prostaglandin receptors E1 and E2) on the expression of the transcription factors, FOXP3 (2A), Tbox21 (2B) and GATA3 (2C) in peripheral blood lymphocytes as determined by Real-Time PCR.

FIGS. 3A-3C graphically depict representative data showing the effect of various concentrations of Thioperamide, an antagonist of Histamine H3 and H4 receptors, on the expression levels of the transcription factors, FOXP3 (2A), Tbox21 (2B) and GATA3 (2C), in anti-CD3/anti-CD28 stimulated peripheral blood lymphocytes as determined by Real-Time PCR.

FIGS. 4A-4C graphically depict representative data showing the effect of various concentrations of Thioperamide, an antagonist of Histamine H3 and H4 receptors, on the production of known cytokines in differentiated Th1 (4A), Th2 (4B) and TGFP1-derived Treg cells (4C).

FIGS. 5A-5C graphically depict representative data showing the effect of various concentrations of Serotonin on the expression levels of the transcription factors, FOXP3 (5A), Tbox21 (5B) and GATA3 (5C), in anti-CD3/anti-CD28 stimulated peripheral blood lymphocytes as determined by Real-Time PCR.

FIG. 6 graphically depicts representative data showing the effect of various concentrations of Serotonin on the proliferation of differentiated Th1, Th2, and TGFβ1-derived Treg cells.

FIGS. 7A-7C graphically depict representative data showing the effect of various concentrations of Serotonin, on the production of known cytokines in differentiated Th1 (7A), Th2 (7B) and TGFβ1-derived Treg cells (7C).

FIGS. 8A-8C graphically depict representative data showing the effect of various concentrations of Rolipram, a PDE4 Inhibitor, on the expression levels of the transcription factors, FOXP3 (8A), Tbox21 (8B) and GATA3 (8C), in anti-CD3/anti-CD28 stimulated peripheral blood lymphocytes as determined by Real-Time PCR.

FIGS. 9A-9C graphically depict representative data showing the effect of various concentrations of Zardaverine, a PDE4D Inhibitor, on the expression levels of the transcription factors, FOXP3 (9A), Tbox21 (9B) and GATA3 (9C), in anti-CD3/anti-CD28 stimulated peripheral blood lymphocytes as determined by Real-Time PCR.

FIGS 10A-10B graphically depict representative data showing the effect of various concentrations of Rolipram (10A), a PDE4 Inhibitor, and Zardaverine (10B), a PDE4D Inhibitor, on the proliferation of differentiated Th1, Th2, and TGFβ1-derived Treg cells.

FIGS. 11A-11C graphically depict representative data showing the effect of various concentrations of Rolipram, a PDE4 Inhibitor, on the production of known cytokines in differentiated Th1 (11A), Th2 (11B) and TGFβ1-derived Treg cells (11C).

FIGS. 12A-12C graphically depict representative data showing the effect of various concentrations of Zardaverine, a PDE4D Inhibitor, on the production of known cytokines in differentiated Th1 (12A), Th2 (12B) and TGFβ1-derived Treg cells (12C).

FIGS. 13A-13B graphically depicts representative data showing the quantitation of Western Blot analysis of protein tyrosine phosphorylation in Th1, Th2, and TGFβ1-derived Treg cells grown in the presence and absence of specific pathway inhibitors.

FIG. 14A graphically depicts representative data showing the effect of the specific P13-Kinase inhibitor LY 294002 on the [³H]thymidine incorporation into TH1, TH2 and Treg cells and FIG. 14B graphically depicts representative data showing the effect of the AKT-specific inhibitor, SH-6 on the [³H]thymidine incorporation into TH1, TH2 and Treg cells.

FIG. 15 is Western Blot analysis demonstrating representative data showing distinct tyrosine phosphorylation profiles in human TH1, TH2 and Treg as compared to the resting T cells and inhibitor treated cells.

FIG. 16 depicts representative data showing the identification of a major phosphorylated protein with an apparent molecular weight of 53 kDa, as a Lck a Src family of protein tyrosine kinases.

FIGS. 17A-17C graphically depicts representative data showing the comparison of the integrated OD values for the tyrosine phosphorylation of Lck protein within Th1, Th2 and Treg cells at 5 (FIG. 17A), 15 (FIG. 17B), and 30 (FIG. 17C) minutes after TCR activation.

FIG. 18 depicts representative data showing the quantitation of the phosphorylated bands observed in the Western Blot analysis of protein tyrosine phosphorylation in Th1, Th2, and TGFβ1-derived Treg cells grown in the presence and absence of specific pathway inhibitors.

FIGS. 19-22 graphically depict representative data showing the pattern of activation and inhibition in selected phosphorylated bands in Th1, Th2 and Treg cells at 5, 15, and 30 minutes after full activation of the TCR (+stim) (FIG. 19) or in the presence of the inhibitors LY 294002 and SH-6 (FIGS. 20 and 21, respectively). The data for each band was normalized and expressed as a ratio to the control value obtained under the full activation of the TCR (+stim). FIG. 22 graphically depicts representative data showing the same data when each band was normalized for LY 294002.

FIGS. 23A-23C and FIGS. 24A-24C graphically depict representative data showing the effect of various concentrations of LY 294002 (FIGS. 23A-23C) and SH-6 (24A-24C) on the expression of the transcription factors, FOXP3 (23A and 24A), Tbox21 (23B and 24B) and GATA3 (23C and 24C) in peripheral blood lymphocytes as determined by Real-Time PCR.

### DETAILED DESCRIPTION OF THE INVENTION

In classical immune responses, effector T cell (Teff) responses dominate over responses of T regulatory cells (Treg) resulting in antigen removal. Tolerance initiates with the same steps as the classical activation pathway (i.e., antigen presentation and T cell activation), but factors including, but not limited to, the abundance of antigen, the means by which it is presented to the T cell, and the relative availability of CD4+ cell help lead to the proliferation of a distinct class of lymphocytes called regulatory T cells. Just as effector T cells mediate classical immune responses, regulatory T cells mediate tolerogenic responses. However, unwanted or misdirected immune responses, such as those associated with allergy, autoimmune diseases, organ rejection, chronic administration of therapeutic proteins and the like, can lead to conditions in the body which are undesirable and which, in some instances, can prove fatal. The dominance or shifting of balance of regulatory T cells over effector T cells results in antigen preservation and immunological tolerance.

The present invention is based, at least in part, on the identification of genes which are expressed differentially between effector T cells (Th1 and Th2) and regulatory T cells. Among the genes preferentially expressed by effector T cells are prostaglandin R2 (GenBank Reference Seq.:NM_{—}000956; GI Accession No.: 31881630; SEQ ID Nos.: 37 and 38) and TGFβ1 (GenBank Reference Seq.:000660; GI Accession No.: 10863872; SEQ ID Nos.: 39 and 40) genes listed in Table 1. Among the genes preferentially expressed by regulatory T cells are the Jagged-1 (GenBank Reference Seq.:NM_{—}000214; GI Accession No.: 4557678; SEQ ID Nos.: 1 and 2), GPR-32 (GenBank Reference Seq.:NM_{—}001506; GI Accession No.: 4504092; SEQ ID Nos.: 3 and 4), CD83 (GenBank Reference Seq.:NM_{—}004233; GI Accession No.: 24475618; SEQ ID Nos.: 5 and 6), CD84 (GenBank Reference Seq.:AF054815; GI Accession No.: 6650105; SEQ ID Nos.: 6 and 7), CD89 (GenBank Reference Seq.:NM_{—}133274; GI Accession No.: 19743864; SEQ ID Nos.: 9 and 10), serotonin R(GenBank Reference Seq.:NM_{—}000869; GI Accession No.: 4504542; SEQ ID Nos.: 11 and 12), BY55 (GenBank Reference Seq.:NM_{—}007053; GI Accession No.: 5901909; SEQ ID Nos.: 13 and 14), serotonin R2C (GenBank Reference Seq.:NM_{—}000868; GI Accession No.: 4504540; SEQ ID Nos.: 15 and 16), GPR63 (GenBank Reference Seq.:NM_{—}030784; GI Accession No.: 13540556; SEQ ID Nos.: 17 and 18), histamine R-H4 (GenBank Reference Seq.:NM_{—}021624; GI Accession No.: 14251204; SEQ ID Nos.: 19 and 20), GPR58 (GI Accession No.: 7657141; SEQ ID Nos.: 21 and 22), EPO-R (GenBank Reference Seq.:NM_{—}000121; GI Accession No.: 4557561; SEQ ID Nos.: 23 and 24), PSG-1 (GenBank Reference Seq.:NM_{—}006905; GI Accession No.: 21361391; SEQ ID Nos.: 25 and 26), PSG-3 (GenBank Reference Seq.:NM_{—}021016; GI Accession No.: 11036637; SEQ ID Nos.: 27 and 28), PSG-6 (GenBank Reference Seq.:NM_{—}002782; GI Accession No.: 7524013; SEQ ID Nos.: 29 and 30), PSG-9 (GenBank Reference Seq.:NM_{—}002784; GI Accession No.: 21314634; SEQ ID Nos.: 31 and 32), PDE-4D (GenBank Reference Seq.:NM_{—}006203; GI Accession No.: 32306512; SEQ ID Nos.: 35 and 36), and PI-3-related kinase (GenBank Reference Seq.:NM_{—}015092; GI Accession No.: 18765738; SEQ ID Nos.: 33 and 34) genes listed in Table 2. At least one of these genes can be modulated according to the methods of the invention.

The nucleic acid molecules or the protein products of these genes can be utilized to modulate immune responses or to identify agents which would be capable of modulating immune response. For example, in one embodiment, at least one effector T cell response can be preferentially modified, e.g., without modulating at least one regulatory T cell response (or modulating such responses in a favorable direction, e.g. through the use of an additional agent or protocol). In another embodiment, at least one regulatory T cell response can be preferentially modulated, e.g., without modulating an effector T cell response (or modulating such responses in a favorable direction, e.g., through the use of an additional agent or protocol). Such modulation results in a shifting or alteration in the balance between tolerance and activation and a modulation in the overall immune response.

The invention also pertains to methods useful in diagnosing, treating or preventing conditions that would benefit from modulating at least one effector T cell function relative to at least one regulatory T cell function or modulating at least one regulatory T cell function relative to at least one effector T cell function in a subject.

The instant methods and compositions are especially useful in the diagnosis, treatment or prevention of: conditions characterized by a too-vigorous effector T cell response to antigens accompanied by a normal or lower than normal regulatory T cell response; conditions characterized by a too-vigorous regulatory T cell response to antigens accompanied by a normal or lower than normal effector T cell response; conditions characterized by a weak effector T cell response accompanied by a normal or higher than normal regulatory T cell response; or in the treatment; conditions characterized by a weak regulatory T cell response which accompanied by a normal or higher than normal effector cell response.

In one embodiment of the invention, at least one molecule preferentially expressed by a regulatory T cell or an effector T cell, e.g., including but not limited to those molecules listed in Table 1 and/or Table 2, may be expressed and used in screening assays, e.g., high throughput screening assays, to identify compounds which would modulate, e.g., upmodulate (mimic or agonize) or downmodulate (antagonize) the function of these proteins. Depending on the cell type in which the protein is preferentially expressed and whether an antagonist or agonist of the expression or activity of the protein is chosen, these compounds would be useful, e.g., in reducing unwanted immune responses (e.g., in transplant rejection) by reducing T effector cell responses while permitting the regulatory arm of the immune system to function and eventually control the immune response in the absence of additional drug treatment or by preferentially increasing regulatory T cell responses while permitting the effector arm of the immune system to clear the antigen.

In one embodiment, to preferentially downmodulate at least one T effector cell response, the expression and/or activity of molecules preferentially associated with T effector cells (e.g., as shown in Table 1) is reduced using an inhibitory compound of the invention. In another embodiment, to preferentially downmodulate at least one T effector cell response the expression and/or activity of molecules preferentially associated with T regulatory cells (e.g., as shown in Table 2) is increased using a stimulatory compound of the invention. In another embodiment, both of these methods can be performed to further shift the balance between T effector cells and T regulatory cells.

There are also situations when it is desirable to preferentially stimulate or enhance at least one T effector cell response, e.g., in the case of immune deficiency, cancer, or infection with a pathogen. For example, immune responses against antigens to which a subject cannot mount a significant immune response, e.g., to an autologous antigen, such as a tumor specific antigen, can be induced by up-modulating T effector cell function. Therefore, compounds of the invention can also be used in increasing immune responses (e.g., to pathogens or cancer cells) by preferentially reducing at least one T regulatory cell responses while permitting the T effector cell responses to function or by preferentially increasing effector T cell responses. To upmodulate immune responses, in one embodiment, the expression and/or activity of molecules preferentially associated with T effector cells (e.g., as shown in Table 1) is increased using a stimulatory compound of the invention. In another embodiment, to upmodulate immune responses the expression and/or activity of molecules preferentially associated with T regulatory cells (e.g., as shown in Table 2) is decreased using an inhibitory compound of the invention. In yet another embodiment, both of these methods are performed to further shift the balance between T effector T cells and T regulatory T cells.

Because the balance of T effector cell and T regulatory cell function also serves to control antibody responses, pathogenic B cell activation could also be reduced using the subject methods leading to treatments (for treatment of, e.g., Myasthenia Gravis, Multiple Sclerosis, Systemic Lupus, or inflammatory bowel syndromes) or enhanced in the case of an immunodeficiency using the methods of the invention.

In one embodiment of the invention, unlike currently used immunomodulators, such as immunosuppressives, the modulatory compositions described herein only need to be administered over a short term course of therapy, rather than an intermediate course of therapy or an extended or prolonged course of therapy, to control unwanted immune responses, because they foster development of a homeostatic immunoregulatory mechanism, i.e., to reset, the balance between activation of regulatory T cells and effector T cells. Since the resulting immunoregulation would be mediated by natural T cell mechanisms, no drugs are needed to maintain immunoregulation once an equilibrium between effector T cells and regulatory T cells is established. Elimination of prolonged or life-long treatment with immunosuppressants will eliminate many, if not all, side effects currently associated with treatment of, for example, autoimmunity and organ grafts.

Before further description of the invention certain terms are, for convenience, described below:

### I. Definitions

As used herein, the term “effector T cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffman, 1989, Annu. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells. As used herein, a “T helper type 2 response” (Th2 response) refers to a response by CD4+ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production).

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4+CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody).

As used herein the phrase, “modulating the balance of regulatory T cell function relative to effector T cell function” or “modulating regulatory T cell function relative to effector T cell function” includes preferentially altering at least one regulatory T cell function (in a population of cells including both T effector cells and T regulatory cells) such that there is a shift in the balance of T effector/T regulatory cell activity as compared to the balance prior to treatment.

As used herein the phrase, “modulating the balance of effector T cell function relative to regulatory T cell function” or “modulating effector T cell function relative to regulatory T cell function” includes preferentially altering at least one effector T cell function (in a population of cells including both T effector cells and T regulatory cells) is altered such that there is a shift in the balance of T effector/T regulatory cell activity as compared to the balance prior to treatment.

As used herein, the term “agent” includes compounds that modulate, e.g., up-modulate or stimulate and down-modulate or inhibit, the expression and/or activity of a molecule of the invention. As used herein the term “inhibitor” or “inhibitory agent” includes agents which inhibit the expression and/or activity of a molecule of the invention. Exemplary inhibitors include antibodies, RNAi, compounds that mediate RNAi (e.g., siRNA), antisense RNA, dominant/negative mutants of molecules of the invention, peptides, and/or peptidomimetics.

The term “stimulator” or “stimulatory agent” includes agents, e.g., agonists, which increase the expression and/or activity of molecules of the invention. Exemplary stimulating agents include active protein and nucleic acid molecules, peptides and peptidomimetics of molecules of the invention. The agents of the invention can directly modulate, i.e., increase or decrease, the expression and/or activity of a molecule of the invention. Exemplary agents are described herein or can be identified using screening assays that select for such compounds, as described in detail below.

For screening assays of the invention, preferably, the “test compound or agent” screened includes molecules that are not known in the art to modulate the balance of T cell activation, e.g., the relative activity of T effector cells as compared to the relative activity of T regulatory cells or vice versa. Preferably, a plurality of agents is tested using the instant methods.

In one embodiment, a screening assay of the invention can be performed in the presence of an activating agent. As used herein, the term “activating agent” includes one or more agents that stimulate T cell activation (e.g., effector functions such as cytokine production, proliferation, and/or lysis of target cells). Exemplary activating agents are known in the art and include, but are not limited to, e.g., mitogens (e.g., phytohemagglutinin or concanavalin A), antibodies that react with the T cell receptor or CD3 (in some cases combined with antigen presenting cells or antibodies that react with CD28), or antigen plus antigen presenting cells.

Preferably, the modulating agents of the invention are used for a short term or course therapy rather than an extended or prolonged course of therapy. As used herein the language “short term or course of therapy” includes a therapeutic regimen that is of relatively short duration relative to the course of the illness being treated. For example a short course of therapy may last between about one week to about eight weeks. In contrast, “an intermediate course of therapy” includes a therapeutic regimen that is of longer duration than a short course of therapy. For example, an intermediate course of therapy can last from more than two months to about four months (e.g., between about eight to about 16 weeks). An “extended or prolonged course of therapy” includes those therapeutic regimens that last longer than about four months, e.g., from about five months on. For example, an extended course of therapy may last from about six months to as long as the illness persists. The appropriateness of one or more of the courses of therapy described above for any one individual can readily be determined by one of ordinary skill in the art. In addition, the treatment appropriate for a subject may be changed over time as required.

As used herein, the term “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2. Tolerance can occur to self antigens or to foreign antigens.

As used herein, the term “T cell” (i.e., T lymphocyte) is intended to include all cells within the T cell lineage, including thymocytes, immature T cells, mature T cells and the like, from a mammal (e.g., human). Preferably, T cells are mature T cells that express either CD4 or CD8, but not both, and a T cell receptor. The various T cell populations described herein can be defined based on their cytokine profiles and their function.

As used herein, the term “naïve T cells” includes T cells that have not been exposed to cognate antigen and so are not activated or memory cells. Naïve T cells are not cycling and human naïve T cells are CD45RA+. If naïve T cells recognize antigen and receive additional signals depending upon but not limited to the amount of antigen, route of administration and timing of administration, they may proliferate and differentiate into various subsets of T cells, e.g. effector T cells.

As used herein, the term “memory T cell” includes lymphocytes which, after exposure to antigen, become functionally quiescent and which are capable of surviving for long periods in the absence of antigen. Human memory T cells are CD45RA−.

The “molecules of the invention” (e.g., nucleic acid or polypeptide molecules) are preferentially expressed (and/or preferentially active in modulating the balance between T effector cells and T regulatory cells) in a particular cell type, e.g., effector T cells or in regulatory T cells. Such molecules may be necessary in the process that leads to differentiation of the cell type and may be expressed prior to or at an early stage of differentiation to the cell type. Such molecules may be secreted by the cell, extracellular (expressed on the cell surface) or expressed intracellularly, and may be involved in a signal transduction pathway that leads to differentiation. Modulator molecules of the invention include molecules of the invention as well as molecules (e.g., drugs) which modulate the expression of a molecule of the invention.

As used herein, the term “T regulatory (Treg) molecule” includes molecules that are preferentially expressed and/or active in regulatory T cells.

For example, in one embodiment, a T regulatory molecule is a secreted protein. Exemplary secreted proteins are pregnancy specific beta-1-glycoprotein 1 (SEQ ID Nos:25 and 26), pregnancy specific beta-1-glycoprotein 3 (SEQ ID Nos:27 and 28), pregnancy specific beta-1-glycoprotein 6 (SEQ ID Nos:29 and 30), pregnancy specific beta-1-glycoprotein 9 (SEQ ID Nos:31 and 32). Pregnancy specific glycoproteins (PSG) in humans constitute a family of 11 closely related glycoproteins (PSG1-8, PSG11-13) belonging to the immunoglobulin superfamily, CEA subfamily. Their function(s) is unknown but are produced in large amounts by the placenta.

In another embodiment, a T regulatory molecule is an extracellular protein. Exemplary extracellular proteins are Jagged-1 (SEQ ID Nos:1 and 2), GPR32 (SEQ ID Nos:3 and 4), CD83 (SEQ ID Nos:5 and 6), CD84 (SEQ ID Nos:7 and 8), CD89 (SEQ ID Nos:9 and 10), serotonin receptor 3A (SEQ ID Nos:11 and 12), natural killer cell receptor BY55 (SEQ ID Nos:13 and 14), serotonin receptor 2C (SEQ ID Nos:15 and 16), GPR63 (SEQ ID Nos:17 and 18), histamine receptor H4 (SEQ ID Nos:19 and 20), GPR58 (SEQ ID Nos:21 and 22), erythropoietin receptor (SEQ ID Nos:23 and 24). Jagged-1 is the human homolog of the Drosophila jagged protein and is the ligand for the receptor Notch 1. Mutations that alter the jagged 1 protein cause Alagille syndrome. Jagged 1 signaling through Notch 1 has been shown to play a role in hematopoiesis. GPR32 is an orphan G protein coupled receptor. CD83 is a leukocyte differentiation antigen and member of the immunoglobulin superfamily. CD83 is a target of the NF-kappaB signaling pathway in B cells and the soluble extracellular domain has been shown to inhibit dendritic cell-mediated T-cell proliferation (Lechmann, M., et al. (2002) Trends Immunol. 23 (6), 273-275). CD84 is a leukocyte differentiation antigen and member of the immunoglobulin superfamily CD84 has been found to be rapidly tyrosine phosphorylated following receptor ligation on activated T cells and ligating CD84 enhances the proliferation of anti-CD3 mAb-stimulated human T cells (Tangye S G, et al. (2003) J Immunol. 171(5):2485-95). CD89 is a leukocyte differentiation antigen and member of the immunoglobulin superfamily. It encodes a receptor for the Fc region of IgA. The receptor is a transmembrane glycoprotein present on the surface of myeloid lineage cells such as neutrophils, monocytes, macrophages, and eosinophils, where it mediates immunologic responses to pathogens. It interacts with IgA-opsonized targets and triggers several immunologic defense processes, including phagocytosis, antibody-dependent cell-mediated cytotoxicity, and stimulation of the release of inflammatory mediators. The serotonin receptor 3A is a biogenic hormone that functions as a neurotransmitter, a hormone, and a mitogen. This receptor is a ligand-gated ion channel, which when activated causes fast, depolarizing responses in neurons. The natural killer cell receptor BY55 is a glycosylphosphatidylinositol (GPI)-anchored cell surface molecule that functions as a co-receptor for T cell receptor signaling in circulating cytotoxic effector T lymphocytes lacking CD28 expression (Nikolova M, et al. (2002) Int Immunol. 14(5):445-51). The serotonin receptor 2C is a biogenic hormone that functions as a neurotransmitter, a hormone, and a mitogen. This receptor mediates its actions by association with G proteins that activate phospatidylinositol-calcium second messenger systems. GPR63 is an orphan G-protein coupled receptor. The histamine receptor H4 belongs to the family of G protein-coupled receptors. HRH4 transcripts were found to be highly expressed in peripheral tissues implicated in inflammatory responses (Coge F, et al. (2001) Biochem Biophys Res Commun. 284(2):301-9). GPR58 is n orphan G-protein coupled receptor. The erythropoietin receptor The erythropoietin receptor is a member of the cytokine receptor family. Upon erythropoietin binding, the erythropoietin receptor activates Jak2 tyrosine kinase which activates different intracellular pathways including: Ras/MAP kinase, phosphatidylinositol 3-kinase and STAT transcription factors. The stimulated erythropoietin receptor appears to have a role in erythroid cell survival.

In yet another embodiment, a T regulatory molecule is an intracellular protein. Preferable intracellular molecules are phosphodiesterase 4D (SEQ ID Nos:35 and 36) and PI-3-kinase-related kinase (SEQ ID Nos:33 and 34). Phosphodiesterase 4D belongs to the cyclic nucleotide phosphodiesterase and is homologous to Drosophila dunce. PDE4D plays a role in the regulation of airway smooth muscle relaxation by catalyzing the hydolysis of cAMP. PI-3-kinase-related kinase is involved in nonsense-mediated mRNA decay (NMD) as part of the mRNA surveillance complex. The protein has kinase activity and is thought to function in NMD by phosphorylating the regulator of nonsense transcripts 1 protein.

As used herein the term “T effector (Teff) molecule” includes molecules that are preferentially expressed and/or preferentially active in effector T cells. For example, in one embodiment, a T effector molecule is a secreted protein. A secreted protein may be actively secreted by the cell or secreted by being shed from the cell surface or cleaved from the membrane. An exemplary secreted protein is Transforming growth factor, beta 1 (TGFβ1) (SEQ ID Nos:39 and 40) TGFβ1 is a potent growth inhibitor of normal and transformed epithelial cells, endothelial cells, fibroblasts, neuronal cells, lymphoid cells and other hematopoietic cell types, hepatocytes, and keratinocytes. TGFβ1 inhibits the proliferation of T-lymphocytes by down-regulating predominantly IL-2 mediated proliferative signals. It also inhibits the growth of natural killer cells in vivo and deactivates macrophages. TGFβ1 blocks the antitumor activity mediated in vivo by IL-2 and transferred lymphokine-activated or tumor infiltrating lymphocytes.

In another embodiment, a T effector molecule is an extracellular protein. An exemplary extracellular protein is Prostaglandin E2 receptor, EP2 subtype (PTGER2) (SEQ ID Nos:37 and 38). PTGER2 is a member of the G protein coupled receptor superfamily that is expressed in peripheral leukocytes with alternative transcripts in spleen and thymus. PTGER2 is the receptor for Prostaglandin E2. The activity of this receptor is mediated by G-S proteins that stimulate adenylate cyclase and subsequently raise cAMP levels.

In yet another embodiment, a T effector molecule is an intracellular protein.

As used herein, the phrase “secreted molecule of the invention, refers to a protein molecule, e.g., a protein consisting of a single polypeptide chain, or an oligomeric protein, e.g., homomeric or heteromeric, which is produced inside of a cell and subsequently exported from the cell.

As used herein, the phrase “extracellular molecule of the invention” refers to a protein molecule, e.g., a protein consisting of a single polypeptide chain, or an oligomeric protein, e.g., homomeric or heteromeric, which is either incorporated into or spans the plasma membrane of a cell.

As used herein, the phrase “intracellular molecule of the invention” refers to a protein molecule, e.g., a protein consisting of a single polypeptide chain, or an oligomeric protein, e.g., homomeric or heteromeric, which is located within the cytoplasm or nucleoplasm of a cell.

In one embodiment, small molecules can be used as test compounds. The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

As used herein, the term “oligonucleotide” includes two or more nucleotides covalently coupled to each other by linkages (e.g., phosphodiester linkages) or substitute linkages.

As used herein, the term “peptide” includes relatively short chains of amino acids linked by peptide bonds. The term “peptidomimetic” includes compounds containing non-peptidic structural elements that are capable of mimicking or antagonizing peptides.

As used herein, the term “reporter gene” includes genes that express a detectable gene product, which may be RNA or protein. Preferred reporter genes are those that are readily detectable. The reporter gene may also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), Proc. Natl. Acad. Sci., USA 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898).

### II. Modulatory Agents

A. Stimulatory Agents

According to a modulatory method of the invention, expression and/or activity of a molecule of the invention is stimulated in a cell by contacting the cell with a stimulatory agent. Examples of such stimulatory agents include active protein and nucleic acid molecules that are introduced into the cell to increase expression and/or activity of a molecule of the invention in the cell.

A preferred stimulatory agent is a nucleic acid molecule encoding a protein product of a molecule of the invention, wherein the nucleic acid molecule is introduced into the cell in a form suitable for expression of the active protein of a molecule of the invention in the cell. To express a protein in a cell, typically a nucleic acid molecule encoding a polypeptide of the invention is first introduced into a recombinant expression vector using standard molecular biology techniques, e.g., as described herein. A nucleic acid molecule encoding a polypeptide of the invention can be obtained, for example, by amplification using the polymerase chain reaction (PCR), using primers based on the nucleotide sequence of the molecule of the invention. Following isolation or amplification of the nucleic acid molecule encoding a polypeptide of the invention, the DNA fragment is introduced into an expression vector and transfected into target cells by standard methods, as described herein.

Variants of the nucleotide sequences described herein which encode a polypeptide which retains biological activity are also embraced by the invention. For example, nucleic acid molecules that hybridize under high stringency conditions with the disclosed nucleic acid molecule. As used herein, the term “hybridizes under high stringency conditions” is intended to describe conditions for hybridization and washing under which nucleotide sequences having substantial homology (e.g., typically greater than 70% homology) to each other remain stably hybridized to each other. A preferred, non-limiting example of high stringency conditions are hybridization in a hybridization buffer that contains 6×sodium chloride/sodium citrate (SSC) at a temperature of about 45° C. for several hours to overnight, followed by one or more washes in a washing buffer containing 0.2×SSC, 0.1% SDS at a temperature of about 50-65° C.

Another aspect of the invention features biologically active portions (i.e., bioactive fragments) of a molecule of the invention, including polypeptide fragments suitable for use in making fusion proteins.

In one embodiment, a molecule of the invention or a bioactive fragment thereof can be obtained from cells or tissue sources by an appropriate purification scheme using standard protein purification techniques. In another embodiment, a molecule of the invention immunogen or bioactive fragment is produced by recombinant DNA techniques. Alternative to recombinant expression, a molecule of the invention or bioactive fragment can be synthesized chemically using standard peptide synthesis techniques. While the following teachings may provide certain specific examples, it is intended that the teachings also apply to other molecules of the invention, as defined herein.

The polypeptide, bioactive fragment or fusion protein, as used herein is preferably “isolated” or “purified”. The terms “isolated” and “purified” are used interchangeably herein. “Isolated” or “purified” means that the polypeptide, bioactive fragment or fusion protein is substantially free of cellular material or other contaminating proteins from the cell or tissue source from which the polypeptide is derived, substantially free of other protein fragments, for example, non-desired fragments in a digestion mixture, or substantially free from chemical precursors or other chemicals when chemically synthesized. The language “substantially free of cellular material” includes preparations in which the polypeptide is separated from other components of the cells from which it is isolated or recombinantly produced. In one embodiment, the language “substantially free of cellular material” includes preparations of polypeptide having less than about 30% (by dry weight) of contaminating protein, more preferably less than about 20% of contaminating protein, still more preferably less than about 10% of contaminating protein, and most preferably less than about 5% contaminating protein. When polypeptide is recombinantly produced, it is also preferably substantially free of culture medium, i.e., culture medium represents less than about 20%, more preferably less than about 10%, and most preferably less than about 5% of the volume of the polypeptide preparation. When polypeptide is produced by, for example, chemical or enzymatic processing from isolated or purified protein, the preparation is preferably free of enzyme reaction components or chemical reaction components and is free of non-desired fragments, i.e., the desired polypeptide represents at least 75% (by dry weight) of the preparation, preferably at least 80%, more preferably at least 85%, and even more preferably at least 90%, 95%, 99% or more or the preparation.

The language “substantially free of chemical precursors or other chemicals” includes preparations of polypeptide in which the polypeptide is separated from chemical precursors or other chemicals which are involved in the synthesis of the polypeptide. In one embodiment, the language “substantially free of chemical precursors or other chemicals” includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of polypeptides of the invention include polypeptides comprising amino acid sequences sufficiently identical to or derived from the amino acid sequence of the polypeptide of the invention which include less amino acids than the full length protein, and exhibit at least one biological activity of the full-length protein. Typically, biologically active portions comprise a domain or motif with at least one activity of the full-length protein. A biologically active portion of a polypeptide of the invention can be a polypeptide which is, for example, 10, 20, 30, 40, 50, 100, 150, 200, 250, 300, 350, 400, 450, 500, 550, 600, 650, 700, 750, 800, 850, 900, 950, 1000 or more amino acids in length. Moreover, other biologically active portions, in which other regions of the protein are deleted, can be prepared by recombinant techniques and evaluated for one or more of the functional activities of a native protein. Mutants can also be utilized as assay reagents, for example, mutants having reduced, enhanced or otherwise altered biological properties identified according to one of the activity assays described herein.

Variants of a polypeptide molecule of the invention which retain biological activity are also embraced by the invention. In one embodiment, such a variant polypeptide has at least about 80%, 85%, 90%, 95%, 98% identity.

To determine the percent identity of two amino acid sequences (or of two nucleotide or amino acid sequences), the sequences are aligned for optimal comparison purposes (e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using the sequence of a polypeptide of the invention or a portion thereof as a query, score=50, wordlength=3.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides chimeric or fusion proteins of the molecules of the invention. As used herein, a “chimeric protein” or “fusion protein” comprises a polypeptide of the invention operatively linked to a different polypeptide. Within a fusion protein, the entire polypeptide of the invention can be present or a bioactive portion of the polypeptide can be present. Such fusion proteins can be used to modify the activity of a molecule of the invention.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety. A nucleic acid molecule encoding a polypeptide of the invention can be cloned into such an expression vector such that the fusion moiety is linked in-frame to the polypeptide of the invention.

Other stimulatory agents that can be used to stimulate the activity of a molecule of the invention protein are chemical compounds that stimulate expression or activity of a molecule of the invention in cells, such as compounds that directly stimulate the protein product of a molecule of the invention and compounds that promote the interaction between a protein product of a molecule of the invention and substrates or target DNA binding sites. Such compounds can be identified using screening assays that select for such compounds, as described in detail below.

B. Inhibitory Agents

Inhibitory agents of the invention can be, for example, intracellular binding molecules that act to inhibit the expression or activity of a molecule of the invention. For molecules that are expressed intracellularly, intracellular binding molecules can be used to modulate expression and/or activity. As used herein, the term “intracellular binding molecule” is intended to include molecules that act intracellularly to inhibit the expression or activity of a protein by binding to the protein itself, to a nucleic acid (e.g., an mRNA molecule) that encodes the protein or to a target with which the protein normally interacts (e.g., to a DNA target sequence to which the marker binds). Examples of intracellular binding molecules, described in further detail below, include antisense marker nucleic acid molecules (e.g., to inhibit translation of mRNA), intracellular antibodies (e.g., to inhibit the activity of protein) and dominant negative mutants of the marker proteins. In the case of molecules that are secreted or expressed on the cell surface, in addition to inhibition by intracellular binding molecules (e.g., antisense nucleic acid molecules or molecules which mediate RNAi) the activity of such molecules can be inhibited using agents which act outside the cell, e.g., to disrupt the binding between a ligand and its receptor such as antibodies.

In one embodiment, an inhibitory agent of the invention is an antisense nucleic acid molecule that is complementary to a gene encoding a molecule of the invention or to a portion of said gene, or a recombinant expression vector encoding said antisense nucleic acid molecule. The use of antisense nucleic acids to downmodulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA. An antisense nucleic acid molecule for inhibiting the expression of protein in a cell can be designed based upon the nucleotide sequence encoding the protein constructed according to the rules of Watson and Crick base pairing.

An antisense nucleic acid molecule can exist in a variety of different forms. For example, the antisense nucleic acid can be an oligonucleotide that is complementary to only a portion of a gene. An antisense oligonucleotide can be constructed using chemical synthesis procedures known in the art. An antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. To inhibit expression in cells in culture, one or more antisense oligonucleotides can be added to cells in culture media, typically at about 200 μg oligonucleotide/ml.

Alternatively, an antisense nucleic acid molecule can be produced biologically using an expression vector into which a nucleic acid has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. For example, for inducible expression of antisense RNA, an inducible eukaryotic regulatory system, such as the Tet system (e.g., as described in Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313) can be used. The antisense expression vector is prepared as described below for recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique, as described herein for recombinant expression vectors.

In another embodiment, a compound that mediates RNAi can be used to inhibit a molecule of the invention. RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In another embodiment, an antisense nucleic acid for use as an inhibitory agent is a ribozyme. Ribozymes are catalytic RNA molecules with ribonuclease activity which are capable of cleaving a single-stranded nucleic acid, such as an mRNA, to which they have a complementary region (for reviews on ribozymes see e.g., Ohkawa, J. et al. (1995) J. Biochem. 118:251-258; Sigurdsson, S. T. and Eckstein, F. (1995) Trends Biotechno. 13:286-289; Rossi, J. J. (1995) Trends Biotechno. 13:301-306; Kiehntopf, M. et al. (1995) J. Mol. Med. 73:65-71). A ribozyme having specificity for the mRNA of a molecule of the invention can be designed based upon the nucleotide sequence of the molecule of the invention cDNA sequence. For example, a derivative of a Tetrahymena L-19 IVS RNA can be constructed in which the base sequence of the active site is complementary to the base sequence to be cleaved in the mRNA of a molecule of the invention. See for example U.S. Pat. Nos. 4,987,071 and 5,116,742, both by Cech et al. Alternatively, a molecule of the invention mRNA can be used to select a catalytic RNA having a specific ribonuclease activity from a pool of RNA molecules. See for example Bartel, D. and Szostak, J. W. (1993) Science 261: 1411-1418.

A polypeptide molecule of the invention or a portion or fragment of a molecule of the invention, can also be used as an immunogen to generate antibodies that bind a molecule of the invention or that block a molecule of the invention binding using standard techniques for polyclonal and monoclonal antibody preparation. Preferably, the molecule of the invention is a secreted molecule of the invention or an extracellular molecule of the invention. In another embodiment, when the polypeptide is expressed intracellularly, an intracellular antibody can be prepared as described in more detail below.

To make antibodies a full-length polypeptide can be used or, alternatively, the invention provides antigenic peptide fragments for use as immunogens. Preferably, an antigenic fragment comprises at least 8 amino acid residues of the amino acid sequence of a polypeptide of the invention and encompasses an epitope of the polypeptide such that an antibody raised against the peptide forms a specific immune complex with the polypeptide of the invention. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of polypeptides that are located on the surface of the protein, e.g., hydrophilic regions. Such regions can be readily identified using art recognized methods.

An immunogen typically is used to prepare antibodies by immunizing a suitable subject, (e.g., rabbit, goat, mouse or other mammal) with the immunogen. An appropriate immunogenic preparation can contain, for example, recombinantly expressed polypeptide or a chemically synthesized polypeptide. The preparation can further include an adjuvant, such as Freund's complete or incomplete adjuvant, or similar immunostimulatory agent. Immunization of a suitable subject with an immunogenic preparation induces a polyclonal antibody response, respectively.

In one embodiment, inhibitory compounds of the invention are antibodies or modified antibody molecules. The term “antibody” as used herein refers to immunoglobulin molecules and immunologically active portions of immunoglobulin molecules, i.e., molecules that contain an antigen binding site which specifically binds (immunoreacts with) an antigen. Examples of immunologically active portions of immunoglobulin molecules include F(ab) and F(ab′)₂ fragments which can be generated by treating the antibody with an enzyme such as pepsin as well as VH and VL domains that can be cloned from antibody molecules and used to generate modified antigen binding molecules, such as minibodies or diabodies.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope of an antigen. A monoclonal antibody composition thus typically displays a single binding affinity for a particular antigen or polypeptide with which it immunoreacts.

Polyclonal antibodies can be prepared as described above by immunizing a suitable subject with an immunogen. The antibody titer in the immunized subject can be monitored over time by standard techniques, such as with an enzyme linked immunosorbent assay (ELISA) using immobilized antigen. If desired, the antibody molecules can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the antigen.

Any of the many well known protocols used for fusing lymphocytes and immortalized cell lines can be applied for the purpose of generating an monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind to the antigen, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with an antigen to thereby isolate immunoglobulin library members that bind the antigen. Kits for generating and screening phage display libraries are commercially available (e.g., the Pharmacia Recombinant Phage Antibody System, Catalog No. 27-9400-01; and the Stratagene SurJZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al PCT International Publication No. WO 91/17271; Winter et al PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Another type of inhibitory agent that can be used to inhibit the expression and/or activity of a molecule of the invention in a cell is an intracellular antibody specific for a molecule of the invention, preferably an intracellular molecule of the invention. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Nat. Acad. Sci. USA 91:5075-5079; Chen, S-Y. et al. (1994) Proc. Nat. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of the activity of a molecule of the invention according to the inhibitory methods of the invention, an intracellular antibody that specifically binds the protein product of a molecule of the invention is expressed in the cytoplasm of the cell. To prepare an intracellular antibody expression vector, antibody light and heavy chain cDNAs encoding antibody chains specific for the target protein of interest are isolated, typically from a hybridoma that secretes a monoclonal antibody specific for the molecule of the invention. Hybridomas secreting anti-molecule of the invention monoclonal antibodies, or recombinant monoclonal antibodies, can be prepared as described below. Once a monoclonal antibody specific for the marker protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. To allow for cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit the activity of a molecule of the invention in a cell, the expression vector encoding the intracellular antibody is introduced into the cell by standard transfection methods, as discussed herein.

Yet another form of an inhibitory agent of the invention is an inhibitory form of a polypeptide molecule of the invention, e.g., a dominant negative inhibitor. For example, in one embodiment, an active site (e.g., an enzyme active site or a DNA binding domain) can be mutated. Such dominant negative proteins can be expressed in cells using a recombinant expression vector encoding the protein, which is introduced into the cell by standard transfection methods.

Other inhibitory agents that can be used to inhibit the activity of a marker protein are chemical compounds that directly inhibit marker activity or inhibit the interaction between the marker and target DNA or another protein. Such compounds can be identified using screening assays that select for such compounds, as described in detail below.

### III. Screening Assays

The invention provides methods (also referred to herein as “screening assays”) for identifying modulators, i.e., candidate or test compounds or agents (e.g., peptides, peptidomimetics, small molecules or other drugs) that have a modulatory effect on the molecules of the invention, preferably a secreted molecule of the invention, an intracellular molecule of the invention, or an extracellular molecule of the invention, in effector T cells relative to regulatory T cells or in regulatory T cells relative to effector T cells.

A. Cell Free Assays

In one embodiment, the screening assay can be done in a cell-free format. A molecule of the invention, e.g., a secreted molecule of the invention, e.g., TGFβ1 , is expressed by recombinant methods in host cells and the polypeptide can be isolated from the host cell culture medium using standard methods for purifying polypeptides, for example, by ion-exchange chromatography, gel filtration chromatography, ultrafiltration, electrophoresis, and/or immunoaffinity purification with antibodies specific for a molecule of the invention to produce protein that can be used in a cell free composition. Alternatively, an extract of a molecule of the invention or cells expressing a molecule of the invention can be prepared for use as a cell-free composition.

The molecule of the invention is then contacted with a test compound and the ability of the test compound to bind to a molecule of the invention or bioactive fragment thereof, is determined. Binding of the test compound to a molecule of the invention can be accomplished, for example, by coupling the test compound or a molecule of the invention (e.g., polypeptide or fragment thereof) with an enzymatic or radioisotopic label such that binding of the test compound to the molecule of the invention can be determined by detecting the labeled compound or molecule of the invention in a complex. For example, test compounds or a molecule of the invention (e.g., polypeptides) can be labeled with ¹²⁵I, ³⁵S, ¹⁴C, or ³H, either directly or indirectly, and the radioisotope detected by direct counting of radioemmission or by scintillation counting. Alternatively, test compounds or a molecule of the invention (e.g., polypeptides) can be enzymatically labeled with, for example, horseradish peroxidase, alkaline phosphatase, or luciferase, and the enzymatic label detected by determination of conversion of an appropriate substrate to product.

Binding of the test compound to a molecule of the invention can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules. In a preferred embodiment, the assay includes contacting a polypeptide molecule of the invention or biologically active portion thereof with a target molecule of a molecule of the invention, to form an assay mixture, contacting the assay mixture with a test compound, and determining the ability of the test compound to interact with a polypeptide molecule of the invention, wherein determining the ability of the test compound to interact with a polypeptide molecule of the invention comprises determining the ability of the test compound to preferentially bind to a molecule of the invention or the bioactive portion thereof as compared to a control molecule. In another embodiment, the assay includes contacting a polypeptide molecule of the invention or biologically active portion thereof with a target molecule of a molecule of the invention, to form an assay mixture, contacting the assay mixture with a test compound, and determining the ability of the test compound to modulate binding between a polypeptide molecule of the invention and a known modulator of the polypeptide.

In another embodiment, when a binding partner of the molecule of the invention is known, e.g., a TGFB1 receptor, Notch1, Jak2, EPO, that binding partner can be used in a screening assay to identify modulator compounds.

In another embodiment, the assay is a cell-free assay in which a polypeptide molecule of the invention or bioactive portion thereof is contacted with a test compound and the ability of the test compound to modulate (e.g., stimulate or inhibit) the activity of the polypeptide molecule of the invention or biologically active portion thereof is determined. This embodiment of the invention is particularly useful when the molecule of the invention is an intracellular molecule and its activity can be measured in a cell-free system.

In yet another embodiment, the cell-free assay involves contacting a polypeptide molecule of the invention or biologically active portion thereof with a molecule to which a molecule of the invention binds (e.g., a known binding partner) to form an assay mixture, contacting the assay mixture with a test compound, and determining the ability of the test compound to modulate the activity of the molecule of the invention, as compared to a control compound. The activity of the target molecule can be determined by, for example, detecting induction of a cellular second messenger of the target (i.e., intra-cellular Ca²⁺, diacylglycerol, IP₃, and the like), detecting catalytic/enzymatic activity of the target using an appropriate substrate, detecting the induction of a reporter gene (comprising a target-responsive regulatory element operatively linked to a nucleic acid encoding a detectable marker, e.g., luciferase), or detecting a target-regulated cellular response. For example, PTGER2 is the receptor for PGE2 and the ability of a compound to modulate the binding could be used to identify a modulatory compound. Similarly, the ability of a modulator to effect the binding of TGFβ1 to any of its natural receptors, including but not limited to, Type I, Type II, Type III, and Type IV receptors, TGFβR, and activin receptor like kinase could be used; the ability of a modulator to effect the binding of jagged1 Notch-1 can be assayed; the binding of EPOR to erythropoietin, JAK2, and/or STAT5 can also be used to assess binding.

In one embodiment, the amount of binding of a molecule of the invention to the target molecule in the presence of the test compound is greater than the amount of binding of a molecule of the invention to the target molecule in the absence of the test compound, in which case the test compound is identified as a compound that enhances binding of a molecule of the invention. In another embodiment, the amount of binding of a molecule of the invention to the target molecule in the presence of the test compound is less than the amount of binding of a molecule of the invention to the target molecule in the absence of the test compound, in which case the test compound is identified as a compound that inhibits binding of a molecule of the invention.

Binding of the test compound to a polypeptide molecule of the invention can be determined either directly or indirectly as described above.

In the methods of the invention for identifying test compounds that modulate an interaction between a polypeptide molecule of the invention and a target molecule, the full-length polypeptide molecule of the invention may be used in the method, or, alternatively, only portions of a molecule of the invention may be used. The degree of interaction between a polypeptide molecule of the invention and the target molecule can be determined, for example, by labeling one of the polypeptides with a detectable substance (e.g., a radiolabel), isolating the non-labeled polypeptide and quantitating the amount of detectable substance that has become associated with the non-labeled polypeptide. The assay can be used to identify test compounds that either stimulate or inhibit the interaction between a molecule of the invention protein and a target molecule. A test compound that stimulates the interaction between a polypeptide molecule of the invention and a target molecule, e.g., an agonist, is identified based upon its ability to increase the degree of interaction between a polypeptide molecule of the invention and a target molecule as compared to the degree of interaction in the absence of the test compound. A test compound that inhibits the interaction between a polypeptide molecule of the invention and a target molecule, e.g., an antagonist, is identified based upon its ability to decrease the degree of interaction between a polypeptide molecule of the invention and a target molecule as compared to the degree of interaction in the absence of the compound.

In more than one embodiment of the assays of the present invention it may be desirable to immobilize either a molecule of the invention or a molecule of the invention target molecule, for example, to facilitate separation of complexed from uncomplexed forms of one or both of the polypeptides, or to accommodate automation of the assay. Binding of a test compound to a polypeptide molecule of the invention, or interaction of a polypeptide molecule of the invention with a molecule of the invention target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the polypeptides to be bound to a matrix. For example, glutathione-S-transferase/a molecule of the invention fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target polypeptide or a polypeptide molecule of the invention, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of a molecule of the invention binding or activity determined using standard techniques.

Other techniques for immobilizing polypeptides on matrices can also be used in the screening assays of the invention. For example, either a polypeptide molecule of the invention or a molecule of the invention target molecule can be immobilized utilizing conjugation of biotin and streptavidin. A biotinylated polypeptide molecule of the invention or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with a polypeptide molecule of the invention or target molecules but which do not interfere with binding of a polypeptide molecule of the invention to its target molecule can be derivatized to the wells of the plate, and unbound target or a polypeptide molecule of the invention is trapped in the wells by antibody conjugation. Methods for detecting such complexes, in addition to those described above for the GST-immobilized complexes, include immunodetection of complexes using antibodies reactive with a polypeptide molecule of the invention or target molecule, as well as enzyme-linked assays which rely on detecting an enzymatic activity associated with a polypeptide molecule of the invention or target molecule.

B. Cell-Based Assays

In one embodiment, a cell that naturally expresses or, more preferably, a cell that has been engineered to express a molecule of the invention, for example, by introducing into the cell an expression vector encoding the polypeptide is used in the screening methods of the invention. Alternatively, a polypeptide molecule of the invention (e.g., a cell extract from a molecule of the invention expressing cell or a composition that includes a purified molecule of the invention, either natural or recombinant) can be used.

Compounds that modulate expression and/or activity of a molecule of the invention (or a molecule that acts upstream or downstream of a molecule of the invention) can be identified using various “read-outs.” Methods for detecting alterations in the expression of and/or an expression profile of a molecule of the invention are known in the art and include, for example, a differential display methodology, Northern blot analysis, quantitative RT-PCR, Western blot analysis.

An example of a “read-out” is the use of an indicator cell which can be transfected with an expression vector, incubated in the presence and in the absence of a test compound, and the effect of the compound on the expression of the molecule or on a biological response regulated can be determined. The biological activities include activities determined in vivo, or in vitro, according to standard techniques for each molecule of the invention. A biological activity can be a direct activity or an indirect activity. Examples of such activities include the stimulation of adenylate cyclase and cAMP production by PTGER2, the production of IL-2 stimulated by TGFB 1, inhibition of dendritic cell-mediated T cell proliferation by CD83, antibody-dependent cell-mediated cytotoxicity by CD89 and hydrolysis of cAMP by PDE4D. Adenylate cyclase activity is measured, for example, by enzyme immunoassay utilizing commercially available kits from, for example, Stratagene, Inc., La Jolla, Calif. IL-2, for example, by flow cytomertry (see, McNerlan, S E, et al. (2002) Exp Gerontol 37(2-3):227-34).

In one embodiment one biological activity of a molecule of the invention is modulated, e.g., intracellular second messenger production or cytokine production. In another embodiment, two biological activities of a molecule of the invention are modulated, e.g., cytokine production and intracellular second messenger production.

The ability of a test compound to modulate binding of a molecule of the invention to a target molecule or to bind to itself can also be determined. Determining the ability of the test compound to modulate binding of a molecule of the invention to a target molecule (e.g., a binding partner, e.g., PGE2 for PTGER2; Type I, Type II, Type III, and Type IV receptors, TGFβR, or activin receptor like kinase for TGFβ1; Notch1 for Jagged 1; and erythropoietin binding for erythropoietin receptor) can be accomplished as described above, by, coupling a target molecule of a molecule of the invention with a radioisotope, enzymatic or fluorescent label such that binding of the test compound to a molecule of the invention is determined by detecting the labeled molecule of the invention-target molecule in a complex.

In another embodiment, a different molecule (i.e., a molecule which is not a molecule of the invention) acting upstream or downstream in a pathway involving a molecule of the invention can be included in an indicator composition for use in a screening assay. Non-limiting examples of molecules that may be used as upstream or downstream indicators include, members of the NF-kappa B signaling pathway for CD83, and STAT5 for the erythropoietin receptor. Compounds identified in a screening assay employing such a molecule would also be useful in modulating a molecule of the invention activity, albeit indirectly.

The cells used in the instant assays can be eukaryotic or prokaryotic in origin.

Recombinant expression vectors that can be used for expression of a polypeptide or a non-polypeptide molecule of the invention acting upstream or downstream of the molecule of the invention in the indicator cell are known in the art. In one embodiment, within the expression vector coding sequences are operatively linked to regulatory sequences that allow for inducible or constitutive expression of the polypeptide in the indicator cell (e.g., viral regulatory sequences, such as a cytomegalovirus promoter/enhancer, can be used). Use of a recombinant expression vector that allows for inducible or constitutive expression of the polypeptide in the indicator cell is preferred for identification of compounds that enhance or inhibit the activity of molecules of the invention. In an alternative embodiment, within the expression vector the coding sequences are operatively linked to regulatory sequences of the endogenous gene (i.e., the promoter regulatory region derived from the endogenous a molecule of the invention gene). Use of a recombinant expression vector in which expression is controlled by the endogenous regulatory sequences is preferred for identification of compounds that enhance or inhibit the transcriptional expression of the a molecule of the invention.

In one embodiment, an assay is a cell-based assay in which a cell expressing a molecule of the invention is contacted with a test compound and the ability of the test compound to modulate the activity of the component(s) is determined. The cell, for example, can be of mammalian origin or a yeast cell. The component (e.g., a polypeptide molecule of the invention, or biologically active portion thereof), for example, can be expressed heterologously or native to the cell. Determining the ability of the test compound to modulate the activity of the component can be accomplished by assaying for any of the activities the molecules of the invention as described herein.

For example, determining the ability of the test compound to modulate the activity a polypeptide of the invention can be accomplished by assaying for the activity of, for example, a molecule of the invention or a target molecule thereof. In another embodiment, determining the ability of the test compound to modulate the activity of a polypeptide, or biologically active portion thereof, is accomplished by assaying for the ability to bind a target molecule or a bioactive portion thereof. In a preferred embodiment, the cell which expresses a polypeptide, or biologically active portion thereof, further expresses a target molecule, or biologically active portion thereof. In another preferred embodiment, the cell expresses more than two molecules of the invention or biologically active portions thereof.

According to the cell-based assays for the present invention, determining the ability of the test compound to modulate the activity of a polypeptide or biologically active portion thereof, can be determined by assaying for any of the native activities of a molecule of a polypeptide or by assaying for an indirect activity which is coincident with the activity of a polypeptide, as described herein, for example, in the case of PTGER2, assaying for cell-mediated cytotoxicity or vascular permeability, or by assaying the activity of a protein encoded by a gene having a response element.

Similarly, for TGFβ1, an indirect activity includes, but is not limited to the differentiation of naïve T cells into regulatory T cells or the induction of tolerance.

Other indirect activities of the molecules of the invention include but are not limited to, for example the inhibition of myoblast differentiation by JAG1; phosphorylation of Fc epsilon RI Gamma2 receptor by FCAR; airway smooth muscle relaxation by PDE4D.

Furthermore, determining the ability of the test compound to modulate the activity of a polypeptide or biologically active portion thereof can be determined by assaying for an activity which is not native to the polypeptide, but for which the cell has been recombinantly engineered. For example, the cell can be engineered to express a reporter gene construct that includes DNA encoding a reporter protein operably linked to a gene regulated by a polypeptide of the invention. It is also intended that in preferred embodiments, the cell-based assays of the present invention comprise a final step of identifying the compound as a modulator of a molecule of the invention activity.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired polypeptide in an appropriate host cell. The term regulatory sequence is intended to include promoters, enhancers, polyadenylation signals and other expression control elements. Such regulatory sequences are known to those skilled in the art and are described in Goeddel, Gene Expression Technology: Methods in Enzymology 185, Academic Press, San Diego, Calif. (1990). It should be understood that the design of the expression vector may depend on such factors as the choice of the host cell to be transfected and/or the type and/or amount of polypeptide desired to be expressed.

A variety of reporter genes are known in the art and are suitable for use in the screening assays of the invention. Examples of suitable reporter genes include those which encode chloramphenicol acetyltransferase, beta-galactosidase, alkaline phosphatase or luciferase. Standard methods for measuring the activity of these gene products are known in the art.

In yet another aspect of the invention, a polypeptide molecule of the invention can be used as a “bait protein” in a two-hybrid assay or three-hybrid assay (see, e.g., U.S. Pat. No.5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins which bind to or interact with a molecule of the invention and are involved in the activity of a molecule of the invention. Such a molecule of the invention-target molecules are also likely to be involved in the regulation of cellular activities modulated by a polypeptide molecule of the inventions.

At least one exemplary two-hybrid system is based on the modular nature of most transcription factors, which consist of separable DNA-binding and activation domains. Briefly, the assay utilizes two different DNA constructs. In one construct, the gene that codes for a polypeptide molecule of the invention is fused to a gene encoding the DNA binding domain of a known transcription factor (e.g., GAL-4). In the other construct, a DNA sequence, from a library of DNA sequences, that encode an unidentified protein (“prey” or “sample”) is fused to a gene that codes for the activation domain of the known transcription factor. If the “bait” and the “prey” proteins are able to interact, in vivo, forming a molecule of the invention-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with a polypeptide molecule of the invention.

Another exemplary two-hybrid system, referred to in the art as the CytoTrap™ system, is based in the modular nature of molecules of the Ras signal transduction cascade. Briefly, the assay features a fusion protein comprising the “bait” protein and Son-of-Sevenless (SOS) and the cDNAs for unidentified proteins (the “prey”) in a vector that encodes myristylated target proteins. Expression of an appropriate bait-prey combination results in translocation of SOS to the cell membrane where it activates Ras. Cytoplasmic reconstitution of the Ras signaling pathway allows identification of proteins that interact with the bait protein of interest, for example, a molecule of the invention protein. Additional mammalian two hybrid systems are also known in the art and can be utilized to identify proteins that interact with a molecule of the invention.

In another aspect, the invention pertains to a combination of two or more assays described herein. For example, a modulating agent can be identified using a cell-based or a cell free assay, and the ability of the agent to modulate the activity and/or expression of a molecule of the invention protein can be confirmed in an in vitro system, e.g., in cell culture, or in vivo, e.g., in an animal such as an animal model of inflammation, using art recognized techniques, or as described herein.

In an embodiment of a screening assay of the invention, once a test compound is identified as modulating a molecule of the invention, the effect of the test compound can be assayed for an ability to modulate effector T cell function relative to T regulatory cell function and can be confirmed as an effector T cell modulator, for example, based on measurements of the effects in immune cells, either in vitro (e.g., using cell lines or cells derived from a subject) or in vivo (e.g., using an animal model). Accordingly, the screening methods of the invention can further comprise determining the effect of the compound on at least one T effector cell activity and/or at least one T regulatory activity to thereby confirm that a compound has the desired effect.

In one embodiment, a compound is further assayed for the ability to modulate an activity associated with a T effector cell, e.g., proliferation or cytokine production or cytotoxicity by a T effector cell. In a further embodiment, the ability of a compound is further assayed for the ability to modulate an activity associated with a T regulatory cell, e.g., proliferation or cytokine production by regulatory T cells, the ability to downregulate T effector cells or induce tolerance. For example, determining the ability of a test compound to modulate tolerance can be determined by assaying secondary T cell responses. If the T cells are unresponsive to the subsequent activation attempts, as determined by IL-2 synthesis and/or T cell proliferation, a state of tolerance has been induced, e.g., T regulatory cells have been activated. Alternatively, if IL-2 synthesis is stimulated and T cells proliferate, T effector cells have been activated. See, e.g., Gimmi, C. D. et al. (1993) Proc. Natl. Acad. Sci. USA 90, 6586-6590; and Schwartz (1990) Science, 248, 1349-1356, for example assay systems that can used as the basis for an assay in accordance with the present invention. T cell proliferation can be measured, for example, by assaying [³H] thymidine incorporation and methods to measure protein levels of members of the MAP kinase cascade or activation of the AP-1 complex. Cytokine levels can be assayed by any number of commercially available kits for immunoassays, including but not limited to, Stratagene, Inc., La Jolla, Calif. Tolerized T cells will have decreased IL-2 production when compared with stimulated T cells. Other methods for measuring the diminished activity of tolerized T cells include, without limitation, measuring intracellular calcium mobilization, measuring protein levels of members of the MAP kinase cascade, and/or by measuring the activity of the AP-1 complex of transcription factors in a T cell upon engagement of its T cell receptors.

In another embodiment, an assay for the expansion of a population of T regulatory and/or T effector cells by detecting cells expressing markers associated with one or the other cell population using techniques described herein or known in the art.

Alternatively, a modulator of a molecule of the invention identified as described herein can be used in an animal model to determine the mechanism of action of such a modulator. For example, an agent can be tested in art recognized animal models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856). A modulatory (i.e., stimulatory or inhibitory) agent of the invention can be administered to test animals and the course of the disease in the test animals can then be monitored using standard methods for the particular model being used. Effectiveness of the modulatory agent is evidenced by amelioration of the disease condition in animals treated with the agent as compared to untreated animals (or animals treated with a control agent).

It will be understood that it may be desirable to formulate such compound(s) as pharmaceutical compositions (described supra) prior to contacting them with cells.

In one aspect, cell-based systems, as described herein, may be used to identify agents that may act to modulate effector T cell function relative to T regulatory cell function, for example. For example, such cell systems may be exposed to an agent, suspected of exhibiting an ability to modulate effector T cell function relative to T regulatory cell function, at a sufficient concentration and for a time sufficient to elicit response in the exposed cells. After exposure, the cells are examined to determine whether one or more responses have been altered.

In addition, in one embodiment, the ability of a compound to modulate effector T cell markers and/or effector T cell markers can be measured.

In addition, animal-based disease systems, such as those described herein, may be used to identify agents capable of modulating effector T cell function relative to T regulatory cell function, for example. Such animal models may be used as test substrates for the identification of drugs, pharmaceuticals, therapies and interventions which may be effective in modulating effector T cell function relative to T regulatory cell function. In addition, an agent identified as described herein (e.g., a modulating agent of a molecule of the invention) can be used in an animal model to determine the efficacy, toxicity, or side effects of treatment with such an agent. Alternatively, an agent identified as described herein can be used in an animal model to determine the mechanism of action of such an agent.

Additionally, gene expression patterns may be utilized to assess the ability of an agent to modulate effector T cell function relative to T regulatory cell function. For example, the expression pattern of one or more genes may form part of “an expression profile” or “transcriptional profile” which may be then used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR.

In one embodiment, the sequences of a molecule of the invention may be used as probes and/or PCR primers for the generation and corroboration of such gene expression profiles.

Gene expression profiles may be characterized for known states within the cell or animal-based model systems. Subsequently, these known gene expression profiles may be compared to ascertain the effect a test agent has to modify such gene expression profiles and to cause the profile to more closely resemble that of a more desirable profile.

Furthermore, this invention pertains to uses of novel agents identified by the above-described screening assays for treatments as described herein.

### IV. Diagnostic Assays

The present invention also features diagnostic assays, for determining expression of a molecule of the invention, within the context of a biological sample (e.g., blood, serum, cells, tissue) to thereby determine whether an individual is afflicted with a disease or disorder, or is at risk of developing such a disorder, or for use as a monitoring method to assess treatment efficacy and/or disease remission. The invention also provides for prognostic (or predictive) assays for determining whether an individual is at risk of developing such a disorder (e.g., a disorder associated with expression or activity of a molecule of the invention) or as a method to prevent relapse of disease. Such assays can be used for prognostic or predictive purpose to thereby phophylactically treat an individual prior to the onset of a disease or disorder. A preferred agent for detecting a molecule of the invention protein is an antibody capable of binding to a molecule of the invention protein, preferably an antibody with a detectable label or primers for amplifying a gene encoding a molecule of the invention. The term “biological sample” is intended to include tissues, cells and biological fluids isolated from a subject, as well as tissues, cells and fluids present within a subject. The invention also encompasses kits for the detection of expression or activity of a molecule of the invention in a biological sample in order to assess the balance between T effector cells and T regulatory cells to a particular antigen in the subject. For example, the kit can comprise a labeled compound or agent capable of detecting a molecule of the invention or its activity in a biological sample; means for determining the amount of a molecule of the invention in the sample; and/or means for comparing the amount of a molecule of the invention in the sample with a standard. The compound or agent can be packaged in a suitable container. The kit can further comprise instructions for using the kit.

### V. Test Compounds

The test compounds or agents of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. Mo. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). In a preferred embodiment, the library is a natural product library.

Non limiting exemplary compounds which can be screened for activity include, but are not limited to, peptides, nucleic acids, carbohydrates, small organic molecules, and natural product extract libraries.

Candidate/test compounds or agents include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), 6) mutant forms of molecules of the invention, e.g., dominant negative mutant forms of Teff molecules of the invention, and 7) antisense RNA molecules or molecules that mediate RNAi.

RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion.

Art recognized techniques of structure based drug design can also be used to identify compounds that modulate the expression or activity of one or more markers of the invention.

### VI. Recombinant Expression Vectors

Another aspect of the invention pertains to vectors, preferably expression vectors, for producing protein reagents (e.g., fusion proteins reagents) of the instant invention or for causing a molecule of the invention to be expressed in a cell, e.g., a patient's cell, e.g., in vitro or in vivo. As used herein, the term “vector” refers to a nucleic acid molecule capable of transporting another nucleic acid to which it has been linked. A preferred vector is a “plasmid”, which refers to a circular double stranded DNA loop into which additional DNA segments can be ligated. In the present specification, “plasmid” and “vector” can be used interchangeably as the plasmid is the most commonly used form of vector. Preferred protein reagents include polypeptides or bioactive fragments thereof of molecules of the invention. While the following teachings exemplify polypeptides and/or fragments thereof, it is intended that the teachings also apply to other molecules of the invention or fragments thereof as defined herein.

The recombinant expression vectors of the invention comprise a nucleic acid that encodes a polypeptide of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). The expression vectors can be introduced into host cells to thereby produce proteins, including fusion proteins or peptides. Alternatively, retroviral expression vectors and/or adenoviral expression vectors can be utilized to express the proteins of the present invention.

The recombinant expression vectors of the invention can be designed for expression of polypeptides in prokaryotic or eukaryotic cells. For example, polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Purified fusion proteins are particularly useful in the cell-free assay methodologies of the present invention.

In yet another embodiment, a nucleic acid molecule encoding a polypeptide of the invention is expressed in mammalian cells, for example, for use in the cell-based assays described herein. When used in mammalian cells, the expression vector's control functions are often provided by viral regulatory elements. In another embodiment, the recombinant mammalian expression vector is capable of directing expression of the nucleic acid preferentially in a particular cell type (e.g., tissue-specific regulatory elements are used to express the nucleic acid).

Another aspect of the invention pertains to assay cells into which a recombinant expression vector has been introduced. An assay cell can be prokaryotic or eukaryotic, but preferably is eukaryotic. A preferred assay cell is a T cell, for example, a human T cell. T cells can be derived from human blood and expanded ex vivo prior to use in the assays of the present invention. Vector DNA can be introduced into prokaryotic or eukaryotic cells via conventional transformation or transfection techniques. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

### VII. Methods of the Invention

A. Methods of Use

The modulatory methods of the invention can be performed in vitro (e.g., by culturing the cell with the agent or by introducing the agent into cells in culture) or, alternatively, in vivo (e.g., by administering the agent to a subject or by introducing the agent into cells of a subject, such as by gene therapy).

In one embodiment, a subject is identified as one that would benefit from modulation of the balance between T effector and T regulatory cells prior to treatment to modulate a molecule of the invention. For example, in one embodiment, the relative activity of T regulatory and T effector cells can be measured. In another embodiment, the relative numbers of T effector cells and T regulatory cells can be calculated. In another embodiment, the presence of T effector and T regulatory cells can be detected at a particular site, e.g., the site of a transplant.

In one embodiment, a subject's cells are assayed for the activity and/or expression of one or more of the molecules of the invention prior to treatment with a modulator of a molecule of the invention (identified as described herein) in order to identify the subject as one that would benefit from the modulation of T effector or T regulatory cells.

In another embodiment, a subject can be monitored after treatment with a conventional immunomodulatory reagent to determine whether the patient would benefit from modulation of the balance between T effector and T regulatory cells.

In another embodiment, a modulator of a molecule of the invention is administered to a subject in vivo or in vitro prior to exposure to an antigen or simultaneously with exposure to an antigen, e.g., Factor VIII treatment.

For practicing the modulatory method in vitro, cells can be obtained from a subject by standard methods and incubated (i.e., cultured) in vitro with a modulatory agent of the invention in order to modulate the activity of a molecule of the invention in the cells. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, T cells can be enriched for example, by positive selection using antibodies to T cell surface markers, for example by incubating cells with a specific primary monoclonal antibody (mAb), followed by isolation of cells that bind the mAb using magnetic beads coated with a secondary antibody that binds the primary mAb. Specific cell populations can also be isolated by fluorescence activated cell sorting according to standard methods. If desired, cells treated in vitro with a modulatory agent of the invention can be re-administered to the subject. For administration to a subject, it may be preferable to first remove residual agents in the culture from the cells before administering them to the subject. This can be done for example by a Ficoll/Hypaque gradient centrifugation of the cells. For further discussion of ex vivo genetic modification of cells followed by re-administration to a subject, see also U.S. Pat. No. 5,399,346 by W. F. Anderson et al.

For practicing the modulatory method in vivo in a subject, the modulatory agent can be administered to the subject such that activity of a molecule of the invention in cells of the subject is modulated. The term “subject” is intended to include living organisms in which an immune response can be elicited. Preferred subjects are mammals. Examples of subjects include humans, monkeys, dogs, cats, mice, rats, cows, horses, goats and sheep.

For stimulatory or inhibitory agents that comprise nucleic acids (including recombinant expression vectors encoding marker protein, antisense RNA, intracellular antibodies or dominant negative inhibitors), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan, J. J. et al. (1995) Gene Therapy 2:38-49; San, H. et al. (1993) Human Gene Therapy 4:781-788).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, and Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

In one embodiment, a retroviral expression vector encoding a marker is used to express marker protein in cells in vivo, to thereby stimulate marker protein expression or activity in vivo. Such retroviral vectors can be prepared according to standard methods known in the art (e.g., as discussed above).

A modulatory agent, such as a chemical compound, can be administered to a subject as a pharmaceutical composition. Such compositions typically comprise the modulatory agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described below.

B. Methods of Treatment

Numerous disease conditions associated with a predominant effector T cell function are known and could benefit from modulation of the type of response mounted in the individual suffering from the disease condition. The methods can involve either direct administration of a modulatory agent to a subject in need of such treatment or ex vivo treatment of cells obtained from the subject with an agent followed by re-administration of the cells to the subject. The treatment may be further enhanced by administering other immunomodulatory agents. Application of the immunomodulatory methods of the invention to such diseases is described in further detail below.

Many autoimmune disorders are the result of inappropriate or unwanted activation of T effector cells resulting in the production of cytokines and autoantibodies involved in the pathology of the diseases. In addition, T effector cell function is associated with graft rejection. Allergies are also mediated by T effector cells. Accordingly, when a reduced effector T cell or antibody response is desired, the methods of the invention can be used to downmodulate the expression and/or activity a molecule preferentially associated with T effector cells, e.g., such that at least one T effector cell function is downmodulated relative to at least one T regulatory cell function. In another embodiment, such disorders can be ameliorated by upmodulating the expression and/or activity of a molecule preferentially associated with T regulatory cells, e.g., such that at least one T regulatory cell function is upmodulated relative to at least one T effector cell function.

In contrast, there are conditions that would benefit from enhancing at least one activity of T effector cells and/or downmodulating at least one activity of T regulatory cells. For example, immune effector cells often fail to react effectively with cancer cells. Accordingly, when a enhanced effector T cell or antibody response is desired, the methods of the invention can be used to upmodulate the expression and/or activity a molecule preferentially associated with T effector cells, e.g., such that at least one T effector cell function is upmodulated relative to at least one T regulatory cell function. In another embodiment, such disorders can be ameliorated by downmodulating the expression and/or activity of a molecule preferentially associated with T regulatory cells, e.g., such that at least one T regulatory cell function is downmodulated relative to at least one T effector cell function.

In one embodiment, these modulatory methods can be used in combination with an antigen to either enhance or reduce the immune response to the antigen. For example, T effector cell responses can be enhanced in a vaccine preparation or reduced in order to reduce effector cell responses to a therapeutic protein which much be chronically administered to the subject, e.g., factor VIII.

More specifically, preferentially downregulating at least one activity of the effector T cells relative to modulating at least one activity of regulatory T cell function in a subject is useful, e.g., in situations of tissue, skin and organ transplantation, in graft-versus-host disease (GVHD), or in autoimmune diseases such as systemic lupus erythematosus, and multiple sclerosis. For example, preferentially promoting regulatory T cell function and/or reducing effector T cell function results in reduced tissue destruction in tissue transplantation. Typically, in tissue transplants, rejection of the transplant is initiated through its recognition as foreign by immune cells, followed by an immune reaction that destroys the transplant. The administration of an agent or modulator as described herein, alone or in conjunction with another immunoregulatory agent prior to or at the time of transplantation can modulate effector T cell function as well as regulatory T cell function in a subject.

Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. The efficacy of reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

As used herein, the term “autoimmunity” refers to the condition in which a subject's immune system (e.g., T and B cells) starts reacting against his or her own tissues. Non-limiting examples of autoimmune diseases and disorders having an autoimmune component that may be treated according to the invention include type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

Preferably, inhibition of effector cell function is useful therapeutically in the treatment of allergy and allergic reactions, e.g., by inhibiting IgE production. Inhibition of effector T cell function and/or promotion of regulatory T cell function can be accompanied by exposure to allergen in conjunction with appropriate MHC molecules. Allergic reactions can be systemic or local in nature, depending on the route of entry of the allergen and the pattern of deposition of IgE on mast cells or basophils. Thus, inhibition of effector T cell mediated allergic responses can occur locally or systemically by administration of an agent or inhibitor.

Preferably, inhibition of at lest one effector T cell function may also be important therapeutically in viral infections of immune cells. For example, in the acquired immune deficiency syndrome (AIDS), viral replication is stimulated by immune cell activation. Inhibition of effector T cell function may result in inhibition of viral replication and thereby ameliorate the course of AIDS.

Upregulating T effector cells is also useful in therapy. Upregulation of at least one T effector activity can be useful in enhancing an existing immune response or eliciting an initial immune response. For example, preferably increasing at least one T effector cell activity using agents which stimulate a molecule of the invention in effector T cells is useful in cases of infections with microbes, e.g., bacteria, viruses, or parasites. These would include viral skin diseases such as Herpes or shingles, in which case such an agent can be delivered topically to the skin. In addition, systemic viral diseases such as influenza, the common cold, and encephalitis might be alleviated by the administration of such agents systemically. In another embodiment, expression and/or activity of at least one molecule of the invention associated with T regulatory cells can be downmodulated.

Immunity against a pathogen, e.g., a virus, can be induced by vaccinating with a viral protein along with an agent that activates effector T cell function in an appropriate adjuvant. Nucleic acid vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. 1996. J. Biotechnol. 44:37)). Alternatively, nucleic acid vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms can be used for delivery to mucosal surfaces. (Sizemore et al. (1995) Science. 270:29). Pathogens for which vaccines are useful include hepatitis B, hepatitis C, Epstein-Barr virus, cytomegalovirus, HIV-1, HIV-2, tuberculosis, malaria and schistosomiasis.

In another application, preferential upregulation or enhancement of at least one effector T cell function is useful in the induction of tumor immunity. In another embodiment, the immune response can be stimulated by the transmission of activating signal. For example, immune responses against antigens to which a subject cannot mount a significant immune response, e.g., to an autologous antigen, such as a tumor specific antigens can be induced in this fashion.

The present invention provides for both prophylactic and therapeutic methods of treating a subject at risk of (or susceptible to) a disorder or having a disease, disorder or condition that would benefit from preferentially modulating at least one effector T cell function while having little effect on a T regulatory response and vice versa. Administration of a prophylactic agent can occur prior to the manifestation of symptoms, such that a disease or disorder is prevented or, alternatively, delayed in its progression.

These agents can be administered in vitro (e.g., by contacting the cell with the agent) or, alternatively, in vivo (e.g., by administering the agent to a subject). As such, the present invention provides methods of treating an individual afflicted with a disease or disorder that would benefit from up- or downmodulation of T effector cells or regulatory T cells while not affecting the other subset.

The modulatory agents of the invention can be administered alone or in combination with one or more additional agents. For example, in one embodiment, two agents described herein can be administered to a subject. In another embodiment, an agent described herein can be administered in combination with other immunomodulating agents. Examples of other immunomodulating reagents include antibodies that block a costimulatory signal, (e.g., against CD28, ICOS), antibodies that activate an inhibitory signal via CTLA4, and/or antibodies against other immune cell markers (e.g., against CD40, against CD40 ligand, or against cytokines), fusion proteins (e.g., CTLA4-Fc, PD-1-Fc), and immunosuppressive drugs, (e.g., rapamycin, cyclosporine A or FK506). In certain instances, it may be desirable to further administer other agents that upregulate immune responses, for example, agents which deliver T cell activation signals, in order elicit or augment an immune response.

Unlike current immunosuppressives, agents or inhibitors as described herein, because they would foster development of a homeostatic immunoregulatory mechanism, would require short term administration (e.g., for a period of several weeks to months), rather than prolonged treatment, to control unwanted immune responses. Prolonged treatment with the agent or inhibitor or with a general immunosuppressant is unnecessary as the subject develops a robust regulatory T cell response to antigens (e.g., donor antigens, self antigens) associated with the condition. Because the resulting immunoregulation is mediated by natural T cell mechanisms, no drugs would be needed to maintain immunoregulation once the dominant regulatory T cell response is established. Elimination of life-long treatment with immunosuppressants would eliminate many, if not all, side effects currently associated with treatment of autoimmunity and organ grafts.

In one embodiment, immune responses can be enhanced in an infected patient by removing immune cells from the patient, contacting immune cells in vitro an agent that activates effector T cell function, and reintroducing the in vitro stimulated immune cells into the patient.

### VIII. Pharmaceutical Compositions

Modulatory agents, e.g., inhibitory or stimulatory agents as described herein, can be incorporated into pharmaceutical compositions suitable for administration. Such compositions typically comprise the agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

### IX. Administration of Modulating Agents

Modulating agents of the invention are administered to subjects in a biologically compatible form suitable for pharmaceutical administration in vivo. By “biologically compatible form suitable for administration in vivo” is meant a form of the agent to be administered in which any toxic effects are outweighed by the therapeutic effects of the agent.

Administration of a therapeutically active amount of the therapeutic compositions of the present invention is defined as an amount effective, at dosages and for periods of time necessary to achieve the desired result. For example, a therapeutically active amount of agent may vary according to factors such as the disease state, age, sex, and weight of the individual, and the ability of agent to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agent can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, to administer the agent by other than parenteral administration, it may be desirable to coat, or co-administer the agent with, a material to prevent its inactivation.

Agent can be co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

When the active compound is suitably protected, as described above, the agent can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

This invention is further illustrated by the following examples, which should not be construed as limiting. The contents of all references, patents and published patent applications cited throughout this application, as well as the Figures, are incorporated herein by reference.

### Examples

### Example 1

### Identification of Genes Preferentially Expressed in T Effector Cells or T Regulatory Cells Using Affymetrix™ Gene Chips

Methods

Culture of T Cell Lines

Differentiated cell lines were produced from cells prepared from human cord blood or peripheral blood CD4+CD45RA+ naïve T cells by a variety of methods, including flow cytometry and magnetic bead separations. Purity of the starting populations was >95%. Cells were then stimulated by CD3 and CD28 antibodies in RPMI 1640 with 10% FCS and 1% Human AB serum with defined mixtures of cytokines and neutralizing antibodies to cytokines to produce the differentiated cell types. Th1 cells were produced by culture with IL12 (62 U/ml) and anti-IL4 (0.2 ug/ml); Th2 cells were produced by culture in IL4(145 U/ml) and anti-IL12 (10 ug/ml) and anti-IFNγ (10 ug/ml); and regulatory T cells were produced by culture in TGFβ (32 U/ml), IL9 (42 U/ml), anti-IL4 (10 ug/ml) and anti-IL12 (10 ug/ml) and anti-IFNγ (10 ug/ml). (Note: anti-IL12 was not used in all experiments). All cultures were supplemented with IL2 (65 U/ml) and IL15 (4500 U/ml). Cells were split into larger culture dishes as warranted by cell division. At the conclusion of one round of cell differentiation (7-12 days), cells were harvested for preparation of total RNA for use in the gene chip experiments.

Affymetrix™ Gene Chip Experiment

RNA from each cell type was prepared using the Qiagen™ RNeasy kit as described by the manufacturer. After isolation of high quality total RNA from each cell type, the RNA was biotin labeled and fragmented for use in the Affymetrix™ Gene chip as recommended by Affymetrix™. Briefly, RNA was copied into cDNA using Superscript™ II polymerase and a T7 primer. The complementary strand was then synthesized using E. coli DNA Polymerase 1. The product, dsDNA, was phenol/chloroform extracted and ethanol precipitated. In vitro transcription using Biotinylated nucleosides was then performed to amplify and label the RNA using the ENZO™ Bioarray High Yield RNA transcript labeling kit. The labeled product was cleaned up using the clean-up procedure described with the Qiagen RNeasy kit. Labeled RNA was fragmented by incubation in 200 mM Tris acetate, 500 mM potassium acetate and 150 mM magnesium acetate and the recommended amount was loaded onto the Affymetrix™ Hu133 gene array, chips A and B. Affymetrix™ chips were hybridized as recommended by the manufacturer and washed as recommended in the Affymetrix™ automated chip washer. Following washing and tagging of Biotinylated RNA fragments with fluorochromes, the chips were read in the Affymetrix™ chip reader. For each cell type and each chip all probesets, representing a total of approximately 34,000 human genes, was scored as “present” or “absent” based on statistical analysis of the fluorescent signals on sense and nonsense portions of the chip using Affymetrix™ Microarray Suite software. These “present” and “absent” calls for each probeset, along with the signal strength were imported into Microsoft™ Access databases. Using queries, datafiles of all genes scored present for each cell type were created. Genes which scored present on all cell types were removed from further study using queries. Datafiles of genes which were unique to a cell type were created using queries to select genes which only scored present on Th1, Th2 or regulatory T cells. In addition, datafiles of genes which were only present in the effector (Th1 and Th2) cells but absent in the regulatory T cells or present only in the regulatory T cells but absent in the effector T cells were created.

Examination of these lists of genes identified a number of genes coding for molecules which could be useful for the identification and development of compounds which would specifically target effector T cells while having little or no effect on regulatory T cells and vice versa. Further examination of these lists identified a number of genes coding for molecules useful as modulatory agents of the invention and in the identification of additional modulatory agents through screening assays. Among the genes preferentially expressed in effector T cells relative to regulatory T cells are those genes listed, but not limited to those found in Table 1. Among the genes preferentially expressed in regulatory T cells relative to effector T cells are those genes listed, but not limited to those found in Table 2.

### Example 2

### Effect of TGFβ1 on Transcription Factor Expression of Activated Human Peripheral Blood Lymphocytes (PBL)

This example describes the effect of TGFβ1 on the expression levels of Tbox 21, GATA3 and FOXP3 expression in anti-CD3/anti-CD28 stimulated PBLs. Real-time PCR was used to quantitate the levels of transcription factor mRNA in the presence and absence of TGFβ1.

PBL were stimulated for 72 hours with anti-CD3/anti-CD28 in the presence or absence of TGFβ1 and total RNA was extracted using a QiganRNeasy Mini Kit according to manufacturer's instructions. RNA was stored at minus 80° C.

cDNA was prepared from RNA using the Applied Biosystems High-Capacity cDNA Archive Kit according to manufacturer's instructions.

One μg cDNA was amplified using Applied Biosystems Assays-on-Demand™ Gene Expression products (i.e., TaqMan Universal PCR Mastermix and Assay-on-Demand solution, including marker specific primers) according to the following protocol, in accordance with manufacturer's instructions. Probe/primer reagents for FOXP3, GATA3 and Tbox21 were obtained from Applied Biosystems via the Assay on Demand program.

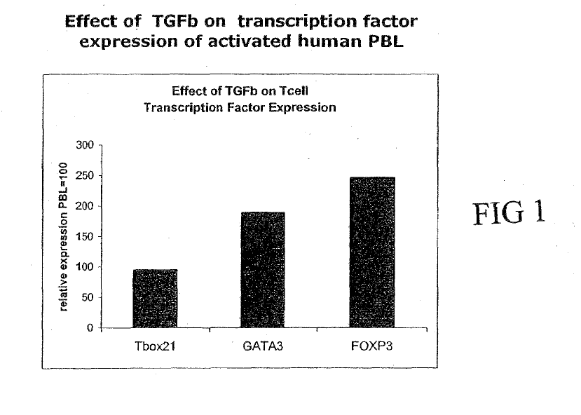

For the QPCR reaction, 2.5 μl Assay on Demand reagent (Applied Biosystems) were added to 25 μl TaqMan Master Mix™ and samples brought to a total volume of 50 μl with RNAse-free water. PCR reactions were run under the following conditions: 50° C. for 1 minute, 95° C. for 10 minutes and 40 cycles of 95° C. for 15 seconds followed by 60° C. for 1 minute. 18 sRNA or β-actin was run with every assay as a control; 2.5 μl of primer/probe mix, 25 μl of TaqMan MasterMix™, 22.5 μl RNAse-free water. Reactants were detected using an Applied Biosystems QPCR instrument (i.e., ABI Program 7000 SDS Sequence Detection System). The relative expression of the transcription factors for both TGFβ1-treated and untreated stimulated PBLs was determined. Data are presented in FIG. 1. Relative expression was calculated assuming that the levels of transcription factor mRNA in stimulated PBL in the absence of added cytokines was 100%.

As seen in FIG. 1, TGFβ1 upregulates FOXP3 expression approximately 2.5-fold relative to an untreated control and upregulates GATA3 approximately 2-fold relative to an untreated control.

### Example 3

### Effect of AH6809, An Antagonist of Prostaglandin E1/E2 Receptors, on Transcription Factor Expression of Activated Human PBL

This example describes the effect of AH6809, an antagonist of Prostaglandin E1/E2 receptors, on the expression levels of the transcription factors, TBX 21, GATA3 and FOXP3, in anti-CD3/anti-CD28 stimulated PBLs.

Real-time PCR was used to quantitate the levels of transcription factor mRNA in the presence and absence of AH6809.

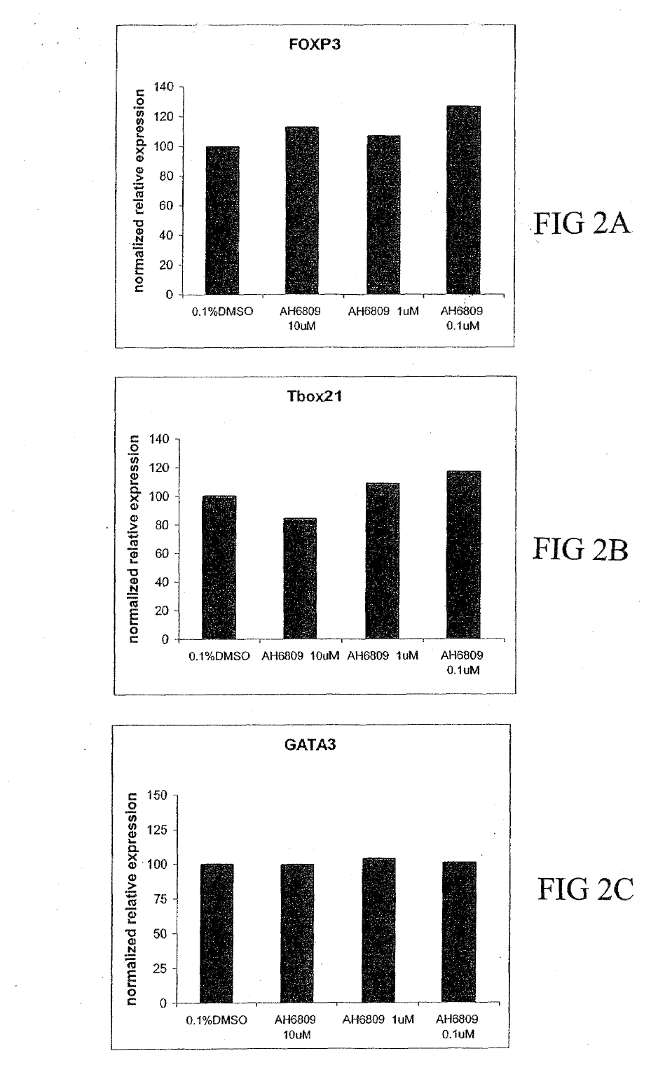

Cells, RNA and cDNA were prepared as described in Example 2, except cells were grown in the presence of AH6809 at 0.1 μM, 1.0 μM and 10 μM or 0.1% DMSO (control). QPCR was performed as described in Example 2 and the relative expression of transcription factor at each concentration of AH6809 was determined. Data are presented in FIGS. 2A, 2B and 2C. Relative expression was calculated assuming that the levels of transcription factor mRNA in stimulated PBL in the presence of DMSO was 100%.

FIG. 2A shows that in the presence of AH6809, there is a trend toward increasing FOXP3 expression with the relative maximal expression found in cells treated with 0.1 μM AH6809. FIG. 2B shows that AH6809 can modulate the expression of Tbox21, e.g. at 0.1 μM, AH6809 expression of Tbox21 was increased relative to untreated control and was decreased at 10 μM AH6809, FIG. 2C demonstrates that GATA3 was unchanged at all concentrations of AH6809 tested.

### Example 4

### Effect of Thioperamide, An Antagonist of Histamine H3 and H4 Receptors, on Transcription Factor Expression of Activated Human PBL

This example describes the effect of Thioperamide, an antagonist of Histamine H3 and H4 receptors, on the expression levels of the transcription factors, TBX21, GATA3 and FOXP3, in anti-CD3/anti-CD28 stimulated PBLs.

Real-time PCR was used to quantitate the levels of transcription factor mRNA in the presence and absence of Thioperamide.

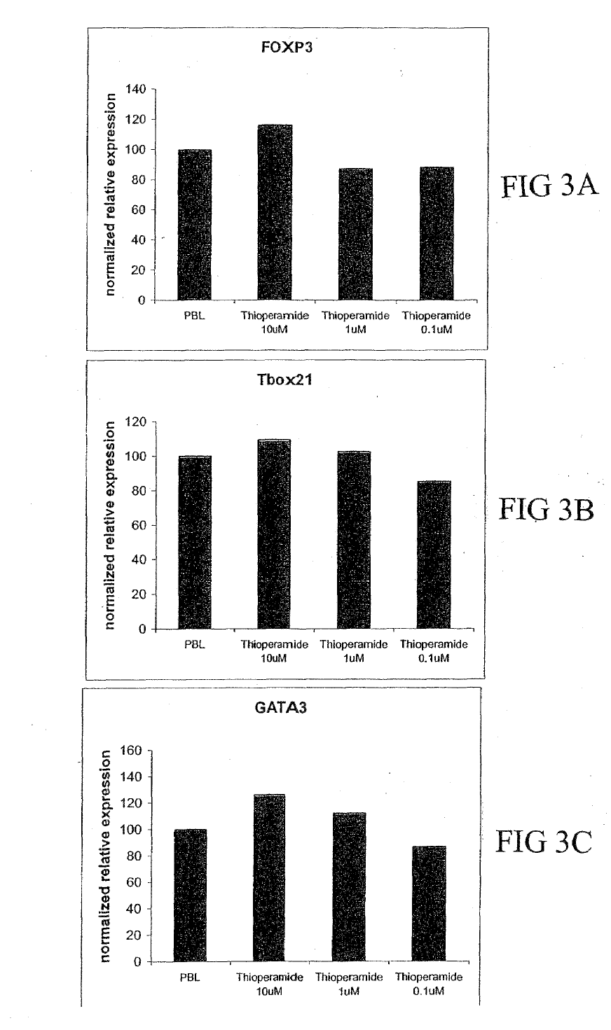

Cells, RNA and cDNA were prepared as described in Example 2, except cells were grown in the presence of Thioperamide at 0.1 μM, 1.0 μM and 10 μM or 0.1% DMSO (control). QPCR was performed as described in Example 2 and the relative expression of transcription factor at each concentration of Thioperamide was determined. Data are presented in FIGS. 3A, 3B and 3C. Relative expression was calculated assuming that the levels of transcription factor mRNA in stimulated PBL in the absence of Thioperamide was 100%.

FIGS. 3A and 3C show that at 10 μM of Thioperamide there was a moderate increase in FOXOP3 and GATA3 expression. FIG. 3B demonstrates that TBX21 was relatively unchanged at all concentrations of Thioperamide tested.

### Example 5

### Effect of Thioperamide, An Antagonist of Histamine H3 and H4 Receptors, on Cytokine Production in Differentiated Cell Types (Th1, Th2 and TGFB1-Derived Treg Cells)

This example describes the effect of Thioperamide on the production of known cytokines in differentiated T cells, specifically Th1, Th2 and TGFβ1-derived Treg cells.

Differentiated cells were prepared as described in Example 1. Varying concentrations (0.1 μM, 1.0 μM and 10 μM) of Thioperamide was added at the time of plating. At the conclusion of one round of cell differentiation (7-12 days), cells were assayed for the production of the cytokines, IL-2, IL-4, IL-5, IL-10, IL-12-p70, IL-13, IFN-γ, TNF-alpha, and TGFβ1, by Searchlight™ technology, a chemiluminescent enzyme-linked immunoabsorbant assay (ELISA) according to the manufacturer's instructions, commercially available from Pierce Biotechnology.

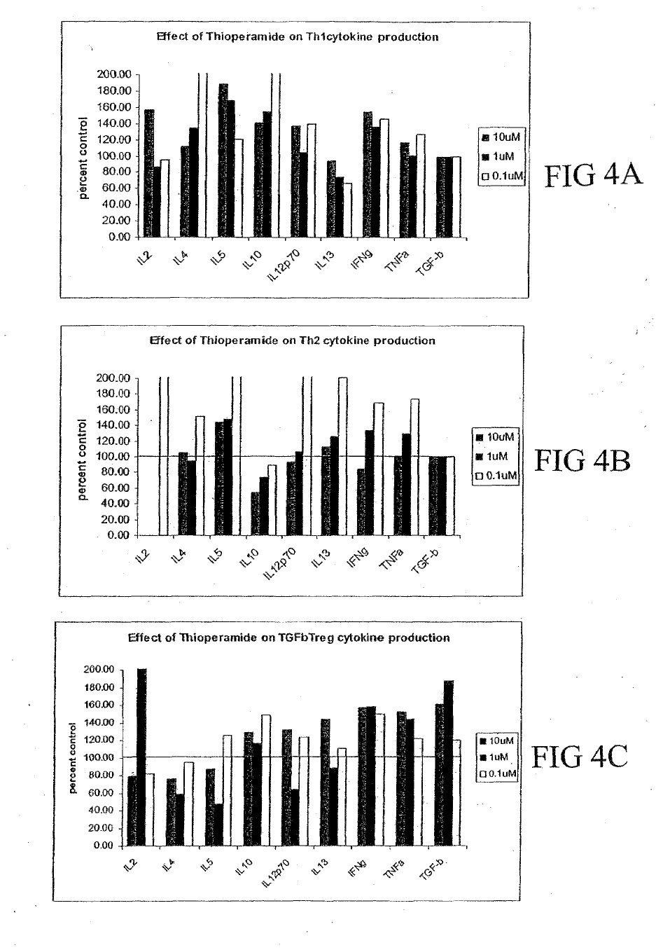

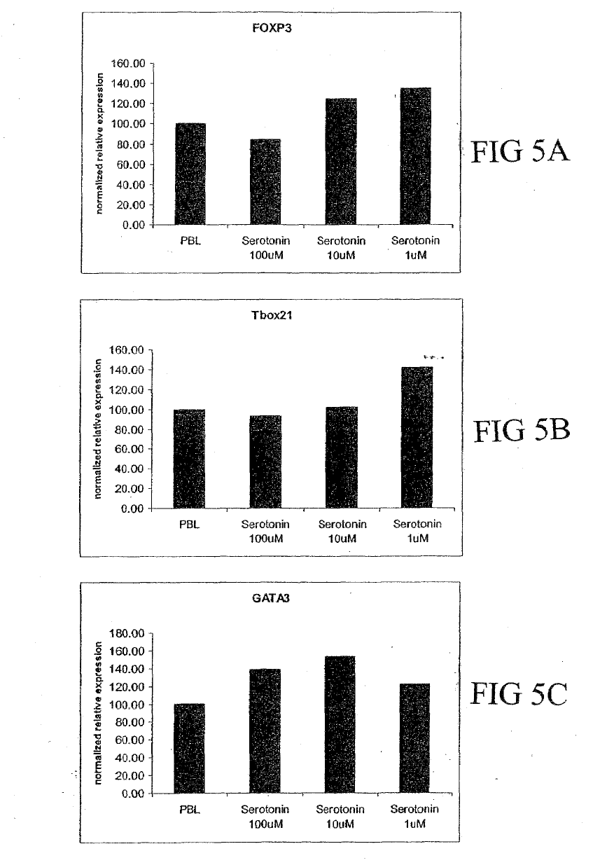

The results of these experiments are shown in FIGS. 4A, 4B, and 4C. Data are plotted as a percent of control (untreated) assuming that the levels of cytokine production in stimulated differentiated cells in the absence of Thioperamide is 100%.

FIG. 4A demonstrates that Thioperamide was able to significantly induce the production of IFN-gamma, and TNF-alpha while significantly reducing the production of IL-13 by Th1 cells. FIG. 4B demonstrates that Thiperamide significantly increased the production of IL-4, IL-5, IL-13, and significantly reduced the production of IL-10 in Th2 cells. In Treg cells, Thioperamide significantly increased the production of IL-2, IL-10, IFN-gamma, and TGFβ1 while thioperamide significantly reduced the production of IL-4, as shown in FIG. 4C.

### Example 6

### Effect of Serotonin on Transcription Factor Expression in Activated Human PBL

This example describes the effect of Serotonin on the expression levels of the transcription factors, TBX21, GATA3 and FOXP3, in anti-CD3/anti-CD28 stimulated PBLs.

Real-time PCR was used to quantitate the levels of transcription factor mRNA in the presence and absence of Serotonin.

Cells, RNA and cDNA were prepared as described in Example 2, except cells were grown in the presence of Serotonin at 1.0 μM, 10.0 μM and 100 μM or in the absence of serotonin. QPCR was performed as described in Example 2 and the relative expression of transcription factor at each concentration of Serotonin was determined. Data are presented in FIGS. 5A, 5B and 5C. Relative expression was calculated assuming that the levels of transcription factor mRNA in stimulated PBL in the absence of serotonin was 100%.

Serotonin was able to increase the expression of each transcription factor relative to untreated control. While each transcription factor was induced by Serotonin, different levels of Serotonin had different effects on the level of the individual transcription factors. For example, FOXP3 was maximally expressed at 10.0 μM and 1.0 μM Serotonin, while Tbox 21 was maximally induced at 1.0 μM and GATA3 was maximally induced at 10.0 μM Serotonin.

### Example 7

### Effect of Serotonin on the Proliferation of Differentiated Cell Types

This example describes the effect of Serotonin at varying concentrations on the proliferation of various T cell types, specifically, Th1, Th2 and TGFβ1-derived Treg cells.

Differentiated cell types were prepared as described in Example 1 then cultured in the presence of anti-CD3 and anti-CD28 for seven days. Cells were subsequently re-stimulated with anti-CD3 and anti-CD28, with the addition of Serotonin at 1, 10 and 100 μM, for three days at which time the cells were counted and the data were plotted as a percent of control (untreated cells).

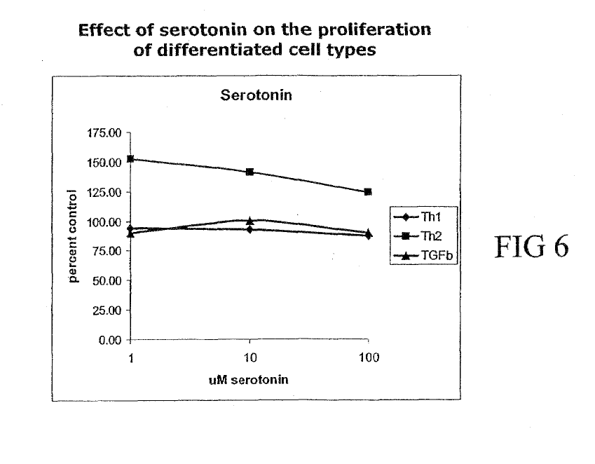

FIG. 6 shows that Serotonin increased the proliferation of Th2 cells by 50% compared to untreated control cells at each concentration tested and had no proliferative effect on Th1 and Treg cells.

### Example 8

### Effect of Serotonin on Cytokine Production in Differentiated Cell Types (Th1, Th2 and TGFβ1-Derived Treg Cells)

This example describes the effect of Serotonin on the production of known cytokines in differentiated T cells, specifically Th1, Th2 and TGFβ1-derived Treg cells.

Differentiated cells were prepared as described in Example 1. Varying concentrations (1.0 μM, 10.0 μM and 100 μM) of Serotonin was added at the time of plating. At the conclusion of one round of cell differentiation (7-12 days), cells were assayed for the production of the cytokines, IL-2, IL-4, IL-5, IL-10, IL-12-p70, IL-13, IFN-γ, TNFα, and TGFβ1, by ELISA as described in Example 5.

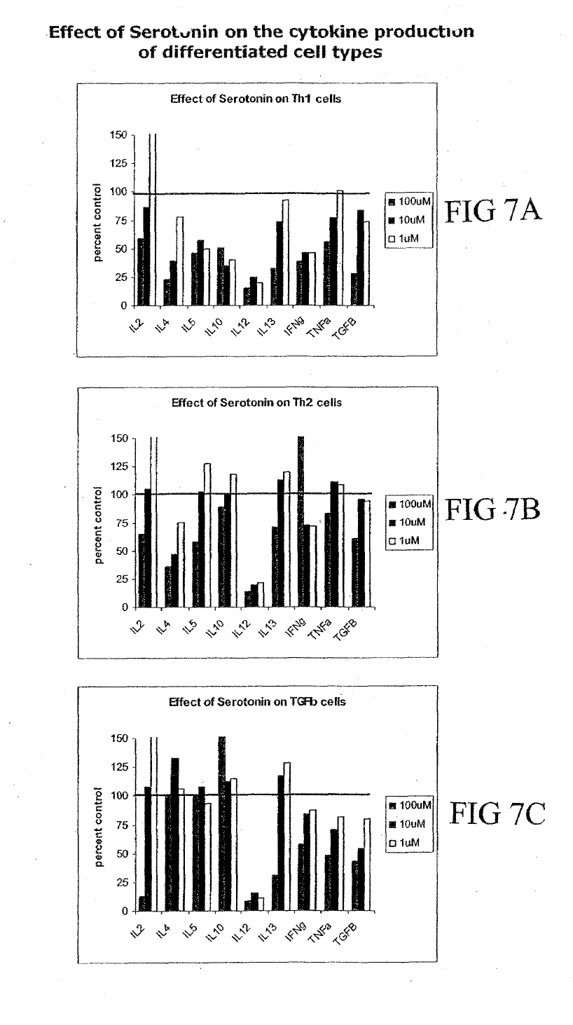

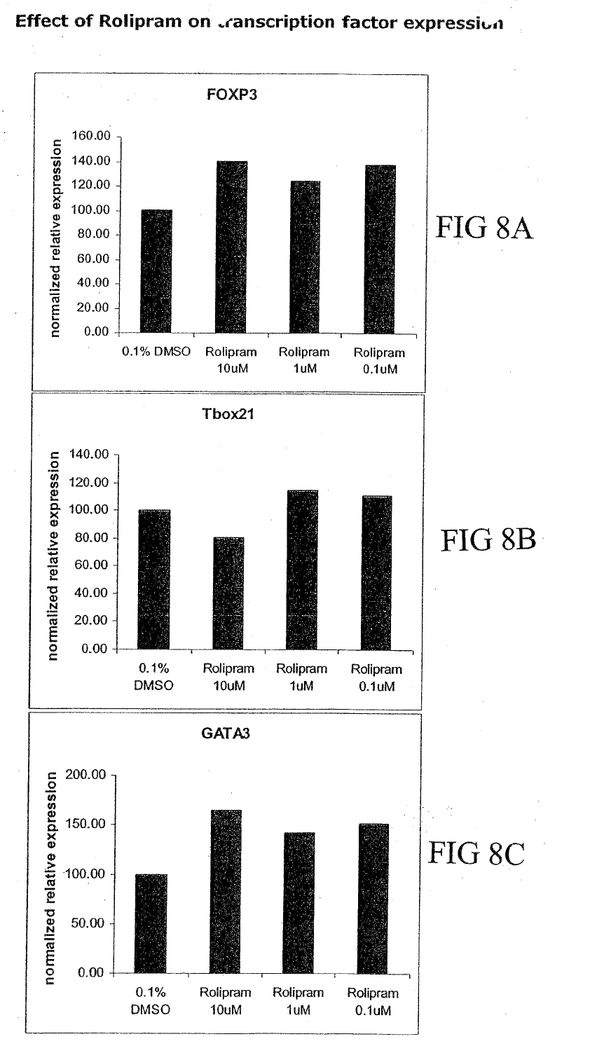

The results of these experiments are shown in FIGS. 7A, 7B, and 7C. Data are plotted as a percent of control (untreated) assuming that the levels of cytokine production in stimulated PBL in the absence of Serotonin is 100%.

FIG. 7A demonstrates that Serotonin significantly reduced the production of IL-2, IL-10, IL-12 IFN-gamma, and TNF-alpha, in Th1 cells. Serotonin significantly reduced the production of, IL-4, IL-5 and IL-13 in Th2 cells and had no effect on IL10 production (FIG. 7B) and as shown in FIG. 7C, Serotonin significantly reduced the production of IL-2, IFN-gamma and TGFβ1 in TGFβ1-derived Treg cells.

### Example 9

### Effect of Rolipram, a PDE4 Inhibitor, and Zardaverine, a PDE4D Inhibitor, on Transcription Factor Expression in Activated Human PBL

This example describes the effects of Rolipram, a PDE4 Inhibitor, and Zardaverine, a PDE4D Inhibitor, on the expression levels of the transcription factors, Tbox 21, GATA3 and FOXP3, in anti-CD3/anti-CD28 stimulated PBLs.

Real-time PCR, as described in Example 2, was used to quantitate the levels of transcription factor mRNA in the presence and absence of Rolipram and Zardaverine.

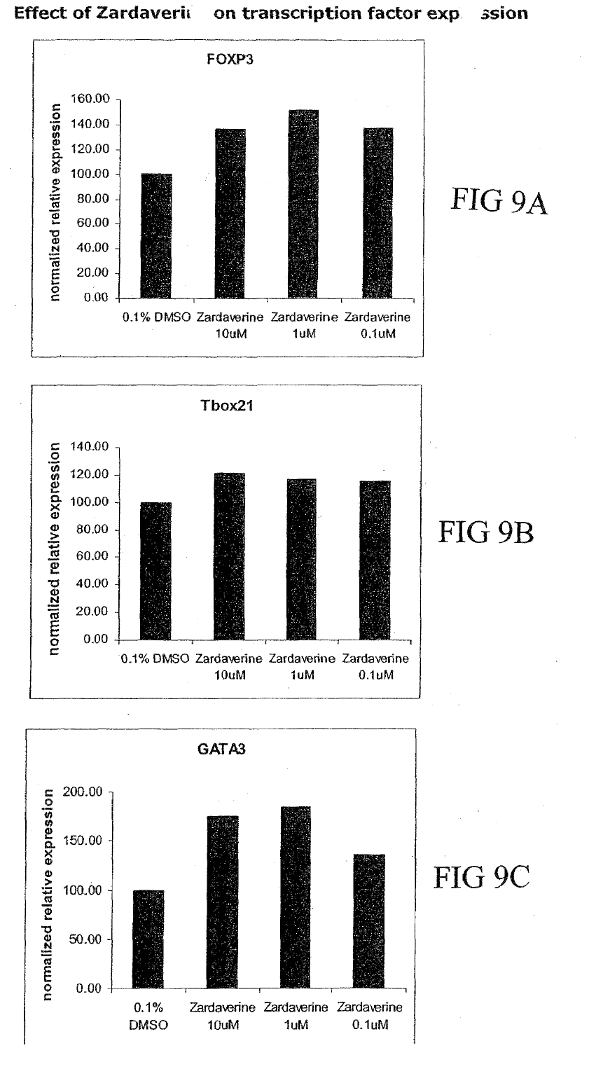

Cells, RNA and cDNA were prepared as described in Example 2, except cells were grown in the presence of Rolipram at 0.1 μM, 1.0 μM and 10 μM or 0.1% DMSO (control) or in the presence of Zardaverine at 0.1 μM, 1.0 μM and 10 μM or 0.1% DMSO (control). QPCR was performed as described in Example 2 and the relative expression of transcription factor at each concentration of Rolipram (FIGS. 8A, 8B, and 8C) or Zardaverine (FIGS. 9A, 9B, and 9C) was determined. Relative expression was calculated assuming that the levels of transcription factor mRNA in stimulated PBL in the presence of DMSO only was 100%.

Treatment with either Rolipram or Zardaverine resulted in an increased expression of FOXOP3 and GATA3 (FIGS. 8A, 8C, 9A, and 9C) while neither of these inhibitors had more than a modest effect on the transcription of Tbox21 (FIGS. 8B and 9B).

### Example 10

### Effect of Rolipram, a PDE4 Inhibitor, and Zardaverine, a PDE4D Inhibitor, on the Proliferation of Differentiated Cell Types

This example describes the effect of Rolipram, a PDE4 Inhibitor, and Zardaverine, a PDE4D Inhibitor, at varying concentrations on the proliferation of various T cell types, specifically, Th1, Th2 and TGFβ1-derived Treg cells.

Differentiated cell types were prepared as described in Example 1 then cultured in the presence of anti-CD3 and anti-CD28 for seven days. Cells were subsequently re-stimulated with anti-CD3 and anti-CD28 (as described in Example 7), with the addition of either Rolipram or Zardaverine at 0.1 μM, 1.0 μM and 10 μM for three days at which time the cells were counted and the data were plotted as a percent of control (untreated cells).

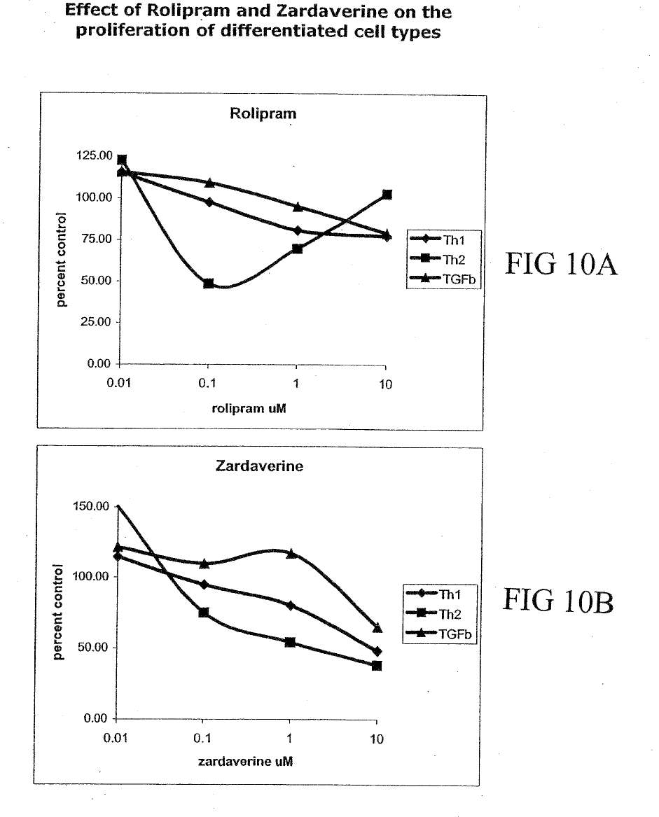

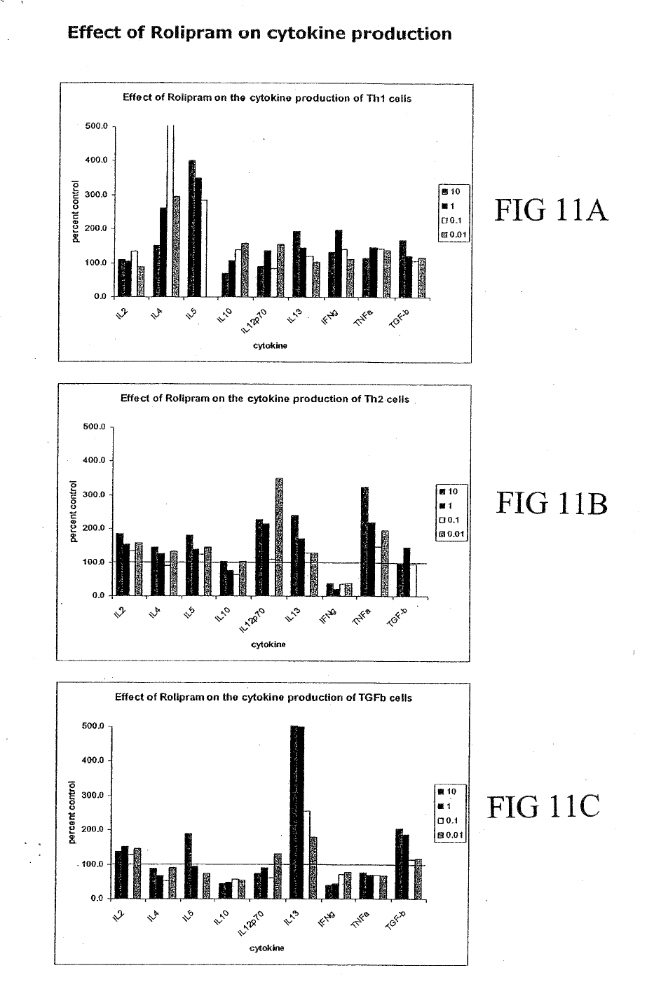

FIGS. 10A and 10B show that while both Rolipram and Zardaverine were able to reduce the proliferation of Th1, Th2 and TGFβ1-derived Treg cells, the proliferation of TGFβ1 -derived Treg cells may have been more strongly affected.

### Example 11

### Effect of Rolipram, a PDE4 Inhibitor, and Zardaverine, a PDE4D Inhibitor, on Cytokine Production in Differentiated Cell Types (Th1, Th2 and TGFβ1-Derived Treg Cells)

This example describes the effect of Rolipram, a PDE4 Inhibitor, and Zardaverine, a PDE4D Inhibitor, on the production of known cytokines in differentiated T cells, specifically Th1, Th2 and TGFβ1-derived Treg cells.

Differentiated cells were prepared as described in Example 1. Varying concentrations (0.1 μM, 1.0 μM and 10.0 μM) of Rolipram or Zardaverine was added at the time of plating. At the conclusion of one round of cell differentiation (7-12 days), cells were assayed for the production of the cytokines, IL-2, IL-4, IL-5, IL-10, IL-12-p70, IL-13, IFN-γ, TNFα, and TGFβ1, by ELISA as described in Example 5.

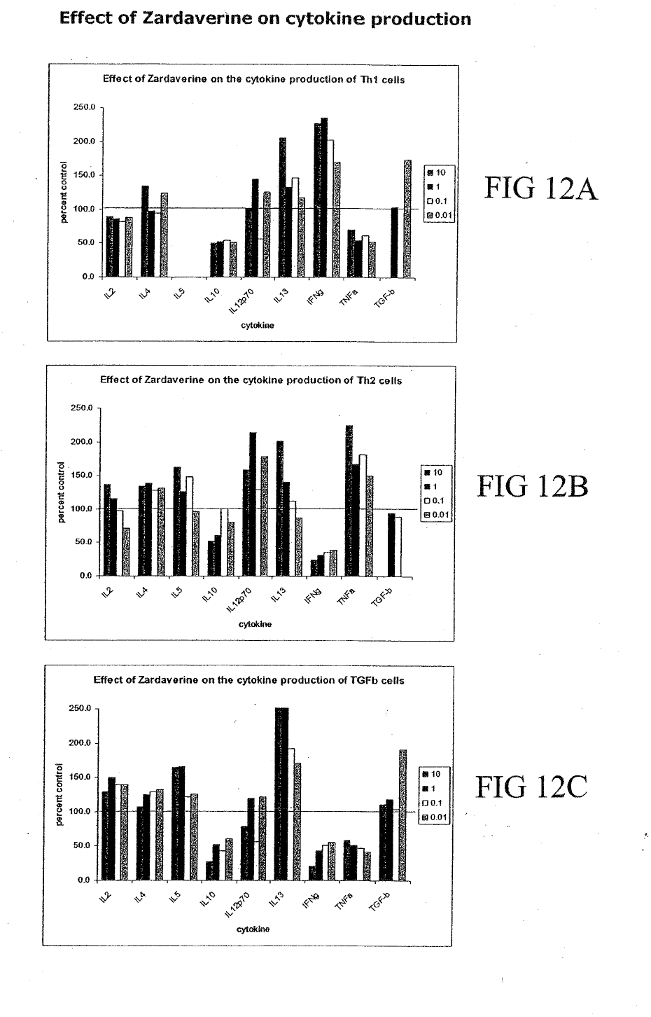

The results of the effect of Rolipram on the production of cytokines is shown in FIGS. 11A, 11B, and 11C, and the results of the effect of Zardaverine on the production of cytokines is shown in FIGS. 12A, 12B, and 12C. Data are plotted as a percent of control (untreated) assuming that the levels of cytokine production in stimulated PBL in the absence of rolipram or zardaverine is 100%.

FIG. 11A demonstrates that Rolipram significantly reduced the production of IL-10 in Th1 cells.

Rolipram significantly increased the production of IL-4, IL-5, IL-13 in Th2 cells (FIG. 11B); and TGFβ1 in TGFβ1-derived Treg cells (FIG. 11C).

FIG. 12A demonstrates that Zardaverine reduced the production of IL-10, and TNF-alpha in Th1 cells; IL-10 in Th2 (FIG. 12B); and IL-10 in TGFβ1-derived Treg cells (FIG. 12C). Zardaverine increased the production of IFN-gamma,in Th1 cells (FIG. 12A); IL-4, IL-5 and IL-13 in Th2 cells (FIG. 12B); and IL-2 and TGFβ1 in TGFβ1-derived Treg cells (FIG. 12C).

### Example 12

### Identification of a Dominant Signaling Pathway Involved in the Differentiation of T Cells

This example relates to the identification of PI-3 kinase and PI-3 kinase-related gene and their signaling pathway as modulators of immunologic tolerance, by directing the differentiation of T cell subsets, including but not limited to effector and regulatory T cells.

Several functional subtypes of CD4+ T cells can be distinguished phenotypically e.g., TH1, TH2 and Treg cells. However, major challenges exist in developing pathway-oriented therapies in order to define the exact contribution of each signaling pathway to the pleiotropic T cell activation responses within these different subtypes of T cells.

Material and Methods

Cell Culture

Human CD4+/CD45RA+ from cord blood has been purchased from AllCell, LLC (cat number, CB02020-4F) and differentiated in vitro under conditions that produce differentiated T cells (TH1, TH2 and Treg) as described in Example 1.

Assessment of [³H] Thymidine Incorporation

Resting, fully differentiated TH1, TH2 and Treg were seeded on 96 well plate coated with anti-CD3 and CD-28. Cells (200,000 per well) were grown in the presence or absence of pathway specific inhibitor for 48 hrs prior to the addition of [³H] thymidine. The cells were then incubated with [³H] thymidine (0.5 μCi/well) for an additional 17 hrs and harvested. [³H] thymidine incorporation was determined by liquid scintillation counting.

Western Blot Analysis

TH1, TH2 and Treg cells were seeded on six well plates coated with anti-CD3 and CD-28. Cells (10×10⁶ per well) were incubated at 37° C. in the presence or absence of pathway specific inhibitor for 5, 15 and 30 min. Cells were lysed in a whole-cell lysis buffer (50 mM Tris-HCl, pH7.2, 0.15 mM NaCl, 50 mM EDTA, 10 mM Na₃VO₄, 5 mM PMSF, 0.115 mM NaF and 1 ug/ml aprotenin).

A total of 5-9 μg of cell lysate protein was run on 4-20% SDS-PAGE, and the proteins were transferred by electroblotting onto polyvinylidine fluoride membrane (Millipore, Bedford, Mass.). The blots were probed with antibodies specific for phosphotyrosine (4G10). Membranes were stripped and reblotted with antibody to Lck. Proteins were visualized using the ECL system (PerkinElmer) after incubating membranes with 2° antibody-conjugated HRP (Amersham Pharmacia Biotech).

Western Blot Quantitation

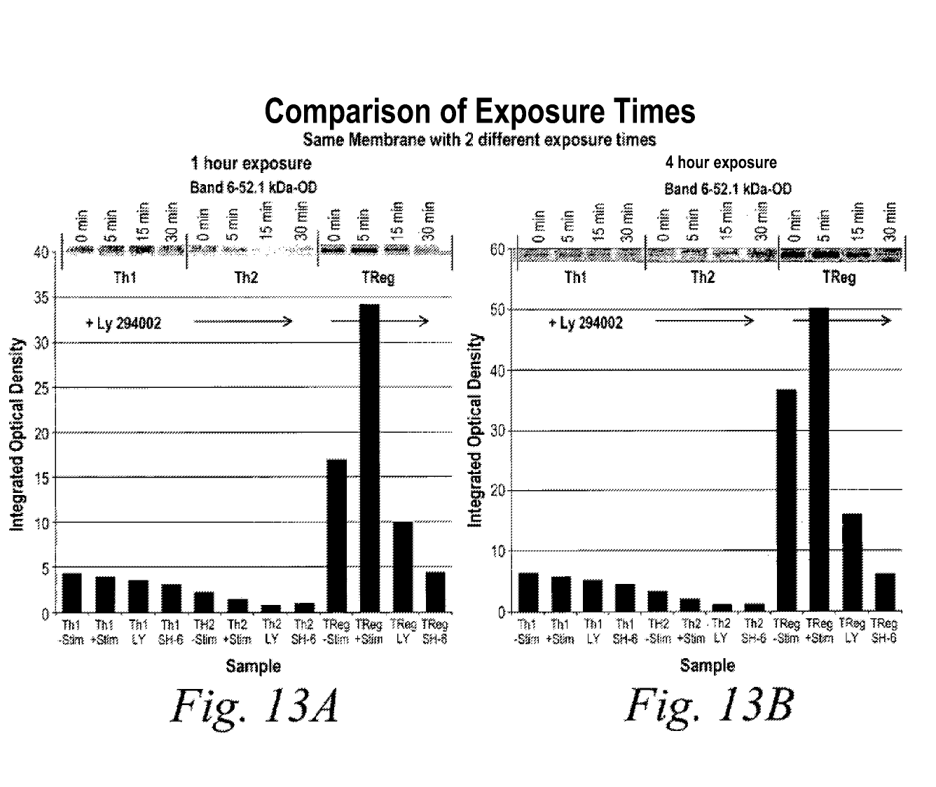

The intensity of the bands was assessed by histogram quantitation and expressed either as a change in OD or as a ratio. Several controls were run to determine the linear range of detection for both the amount of protein loaded, gray scale, and the time of detection. Protein tyrosine phosphorylation was detected within 4.5-8 μg at around 3 hrs as presented in FIGS. 13A (1 hour exposure) and 13B (4 hour exposure), respectively.

Results

Proliferation: PI3-Kinase Pathway

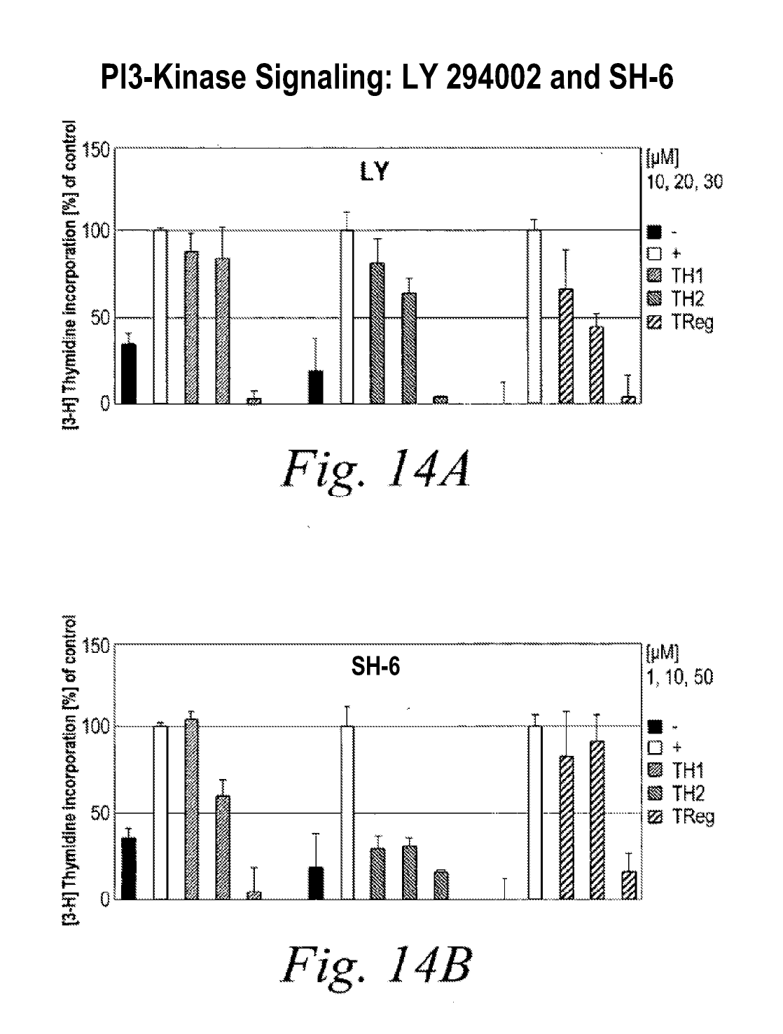

PI3-kinase has been identified as a mediator of proliferative signals in differentiated human T cells. Incubation of cells, in the presence of the specific PI3-Kinase inhibitor LY 294002 significantly reduced [³H]thymidine incorporation into TH1, TH2 and Treg (FIG. 14A). The most profound and dose dependent effect was observed in the Treg subpopulation.

One of the downstream effectors of PI3-kinase is the serine/threonine kinase AKT. An AKT -specific inhibitor, SH-6, was also assessed for its effect again on [3H]thymidine incorporation. As demonstrated in FIG. 14B, 50 μM inhibited proliferation in all three groups of cells analyzed, however, the TH2 group was most affected.

TCR Activation: PI3-Kinase Pathway

Upon T cell receptor (TCR) activation, tyrosine phosphoryaltion of cellular proteins was analyzed by anti-phosphotyrosine Western blot analysis. Using scanning densitometry the apparent molecular weight and integrated OD of the band of interest was determined.

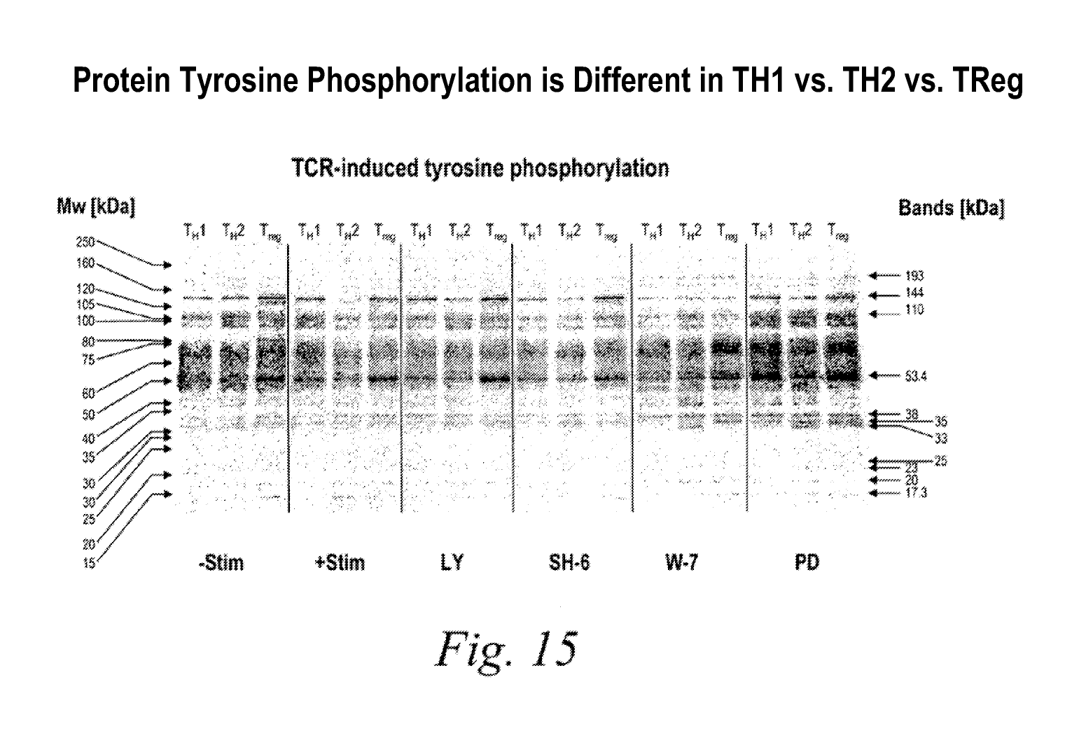

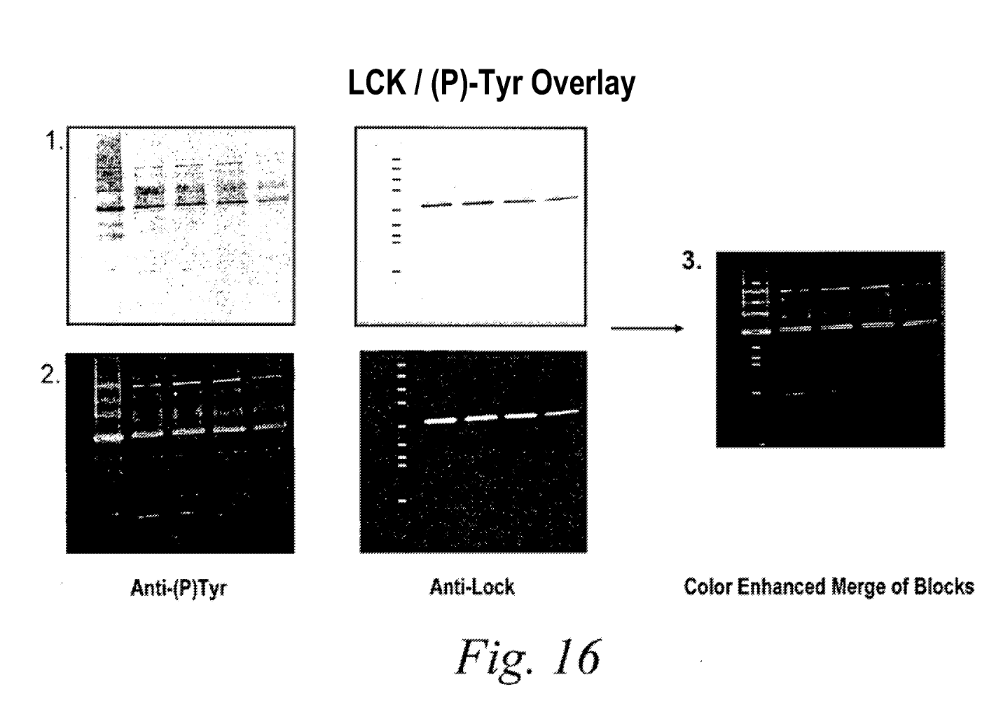

As shown in FIG. 15 a distinct tyrosine phosphorylation profile was observed in human TH1, TH2 and Treg as compared to the resting T cells and inhibitor treated cells.

Identification of Major Phosphorylated Bands

Some of the protein bands were further identified. Striping and reprobing of the original phospho-tyrosine blot with the anti-Lck antibody allowed the identification of a band with an apparent molecular weight of 53 kDa, as a Lck, a Src family of protein tyrosine kinases (FIG. 16).

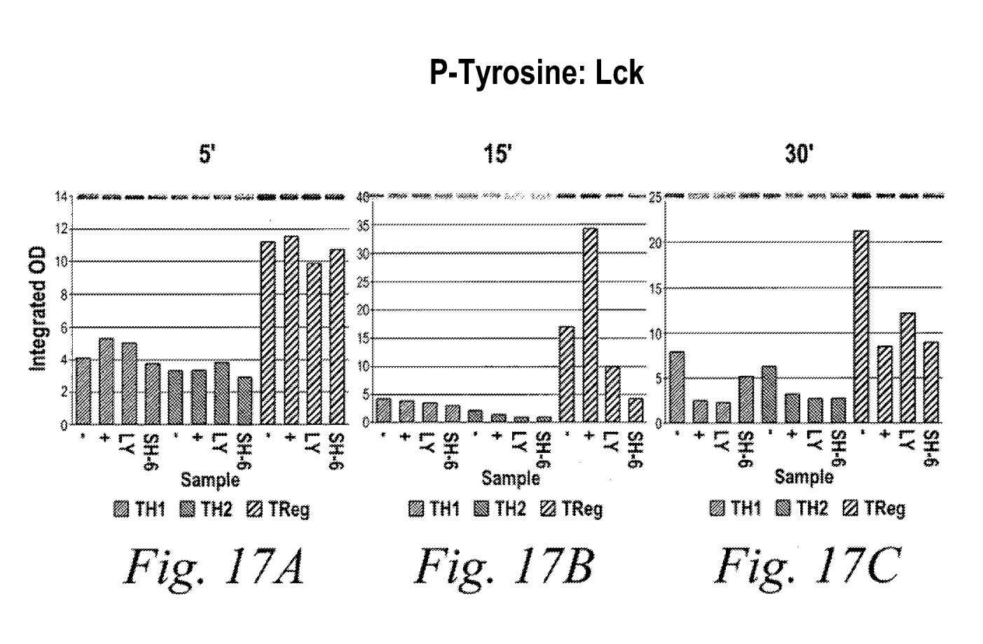

The high-stoichiometric association of Lck with CD4 and CD8 is important for its function in T cells. FIGS. 17A, 17B, and 17C compares the integrated OD value for the tyrosine phosphorylation of Lck protein within TH1, TH2 and Treg at cells at 5 (FIG. 17A), 15 (FIG. 17B), and 30 (FIG. 17C) minutes after TCR activation. The basal level of phosphorylation of Lck in Treg cells was significantly higher than in TH1 or TH2 cells.

LY294002 and SH6 significantly attenuated the extent of Lck phosporylation at 15 min for Treg (FIG. 17B). This inhibitory effect was specific for Treg cells.

Comparative Analysis of Tyrosine Phosphorylation

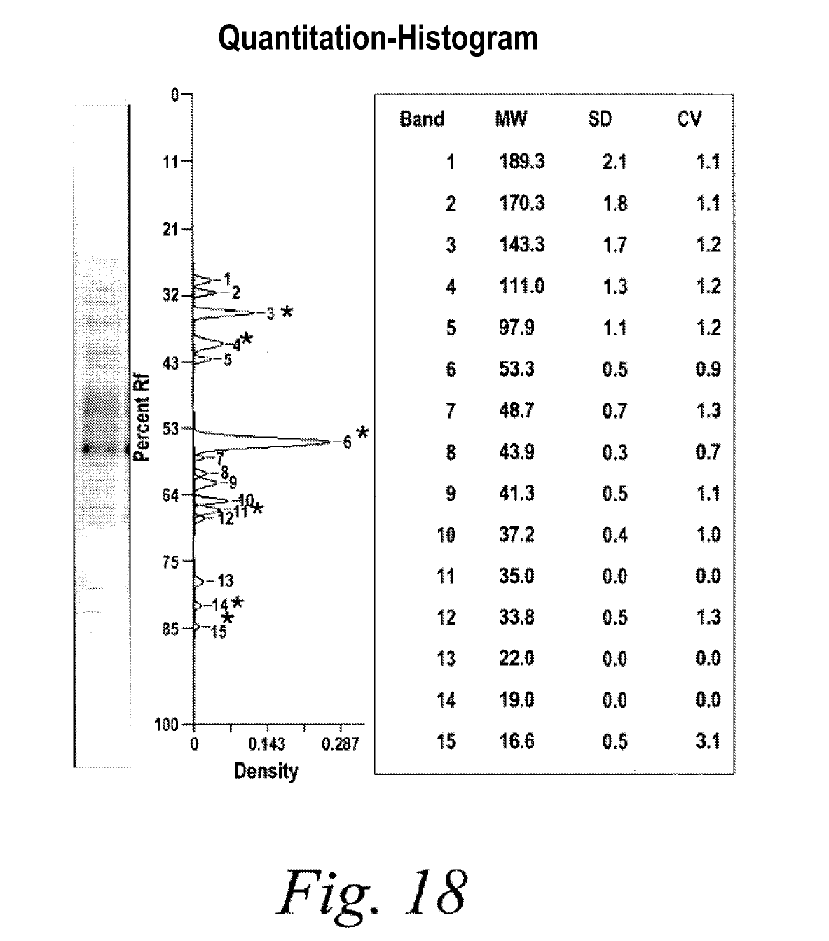

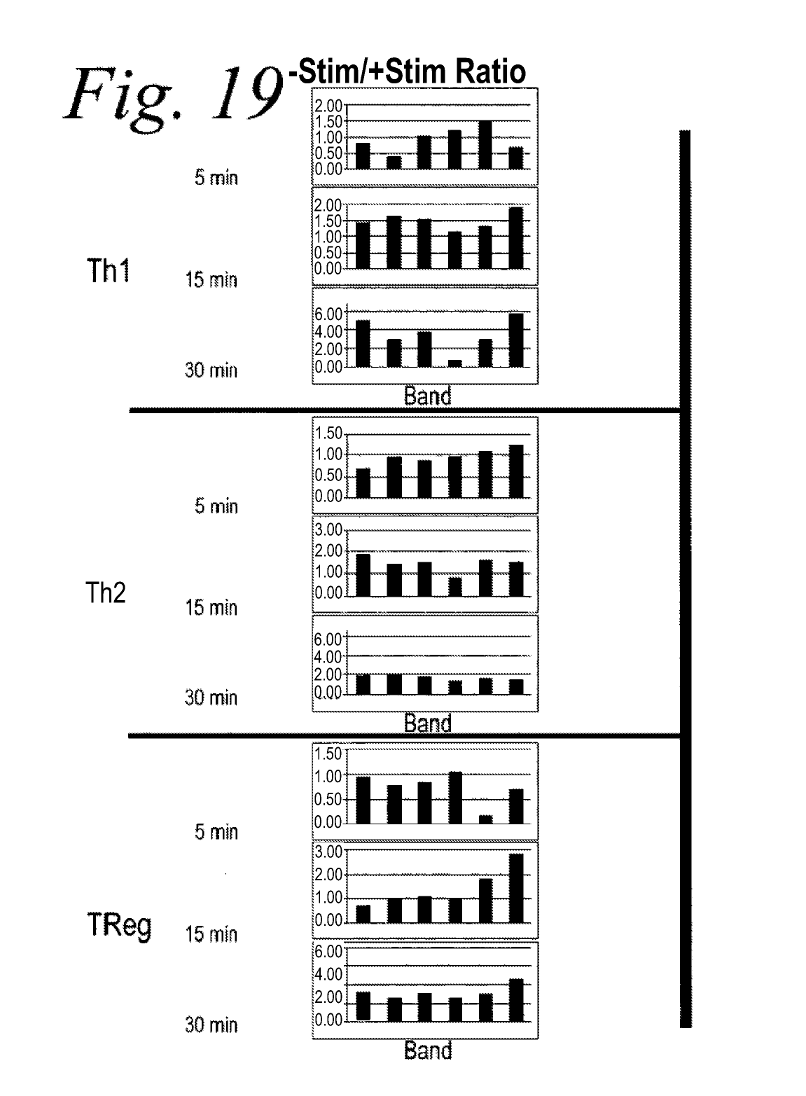

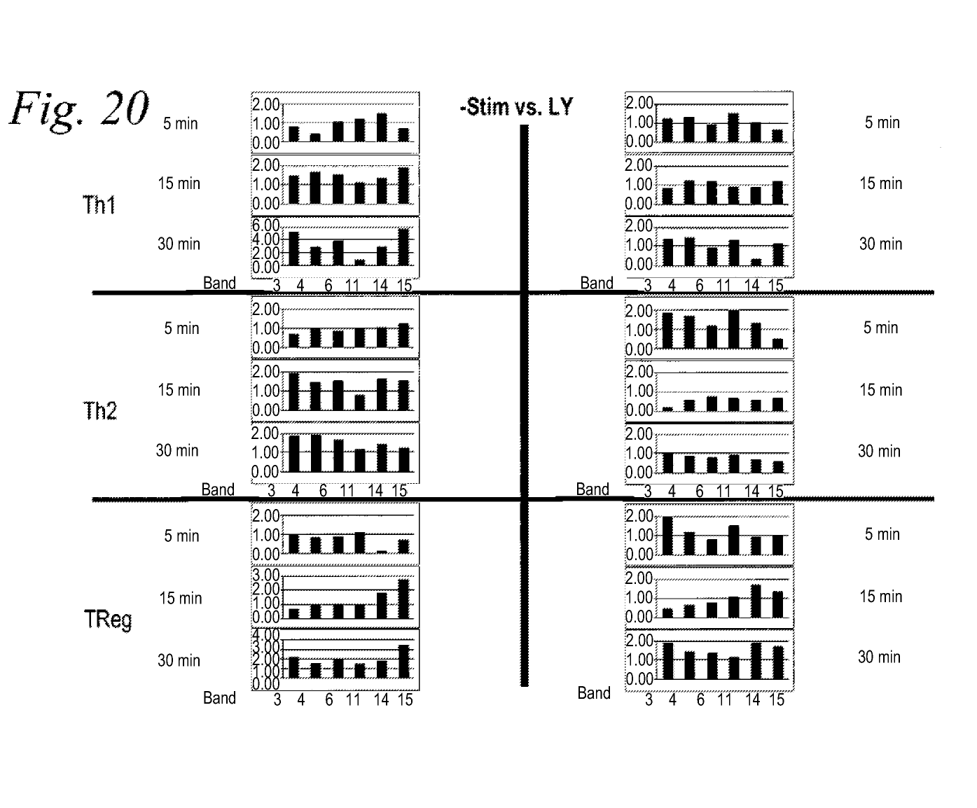

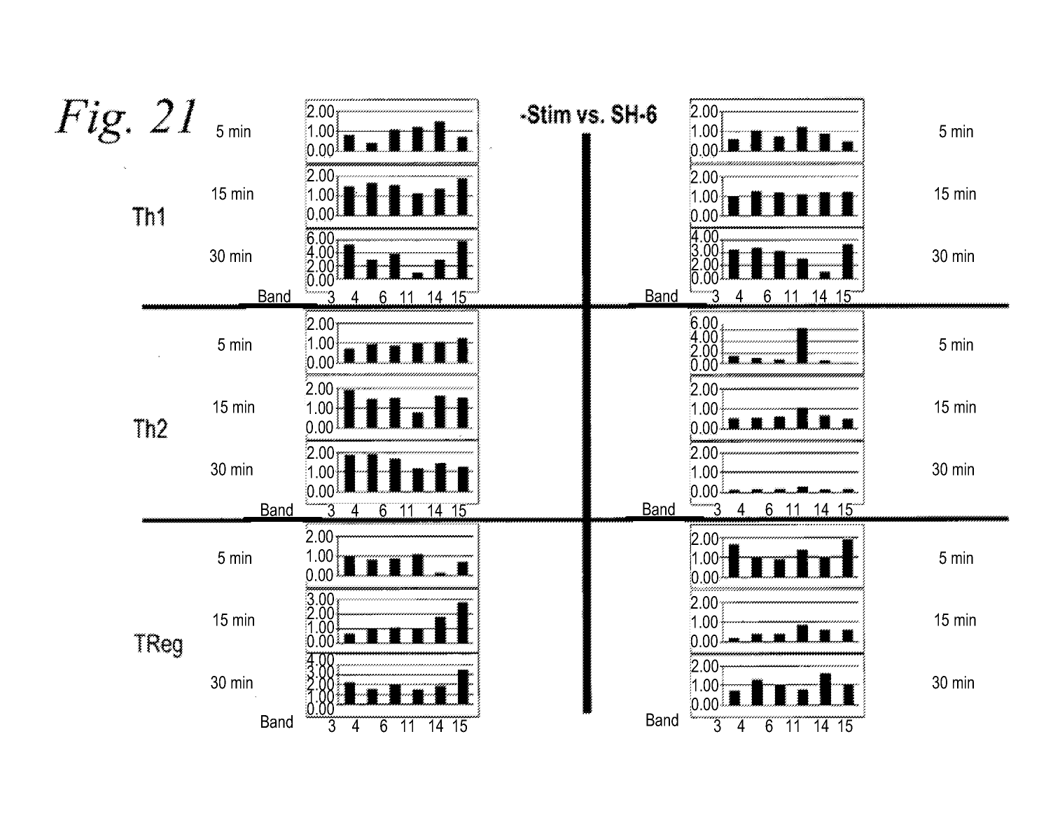

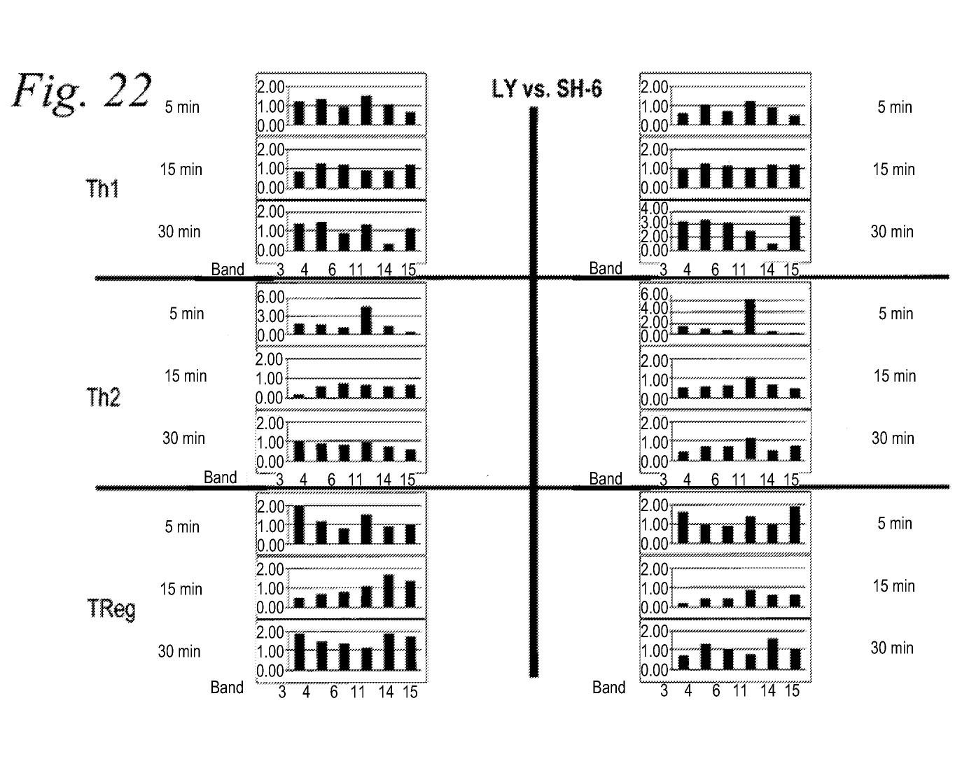

As shown in FIG. 15, several protein bands were the subject of the phosphorylation event. For further comparative analysis, the bands 3,4,6,11,14 and 15 with apparent molecular weights of (kDa) 143, 111, 53, 35, 19 and 15 were chosen for further analysis (FIG. 18) in order to compare the pattern of activation and inhibition. The data for each band was normalized and expressed as a ratio to the control value obtained under the full activation of the TCR (+stim) (FIG. 19) or in the presence of inhibitors (FIGS. 20 and 21, respectively). The data presented highlight the importance of the PI3-kinase pathway, as well as its different input on each subset of T cells. A nearly identical trend has been observed in the presence of SH-6, an inhibitor of AKT downstream of PI-3 kinase (FIG. 22).

Effect of PI3-Kinase Inhibitors on the Expression of Transcription Factors

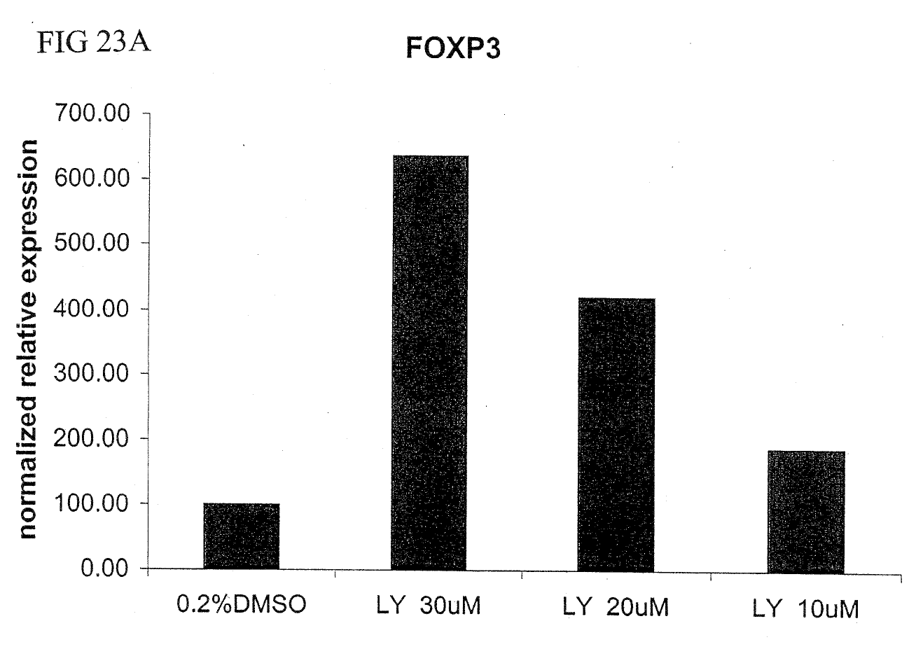

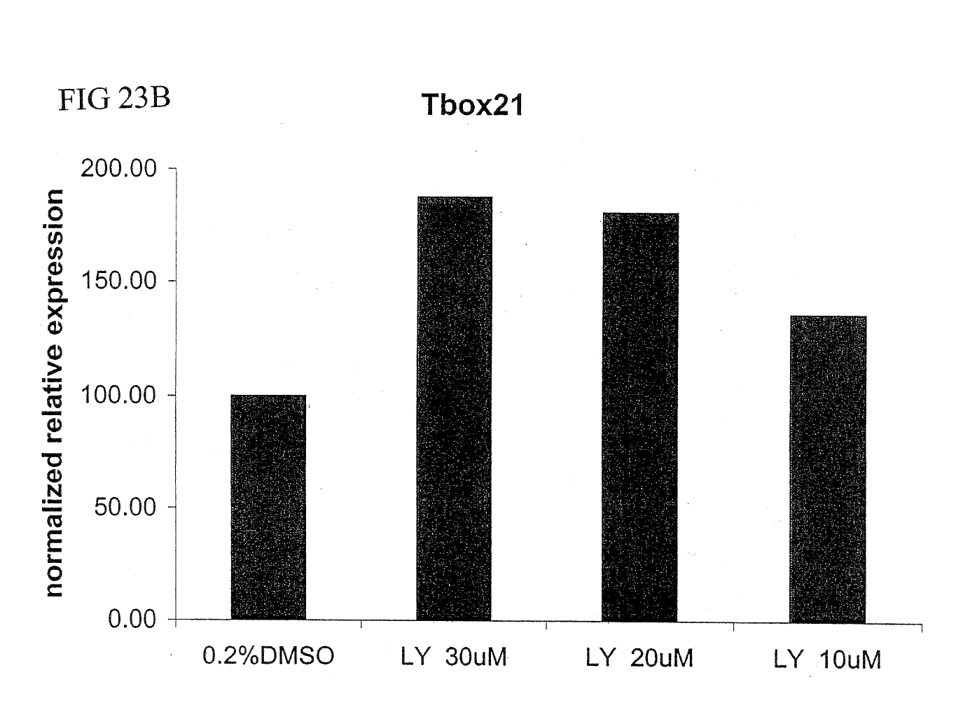

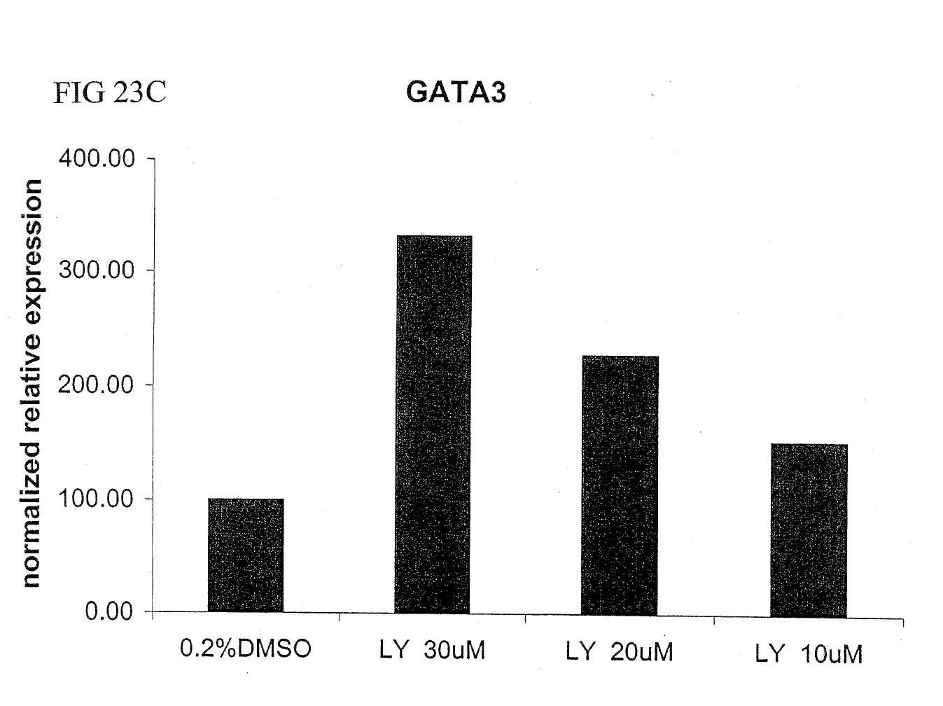

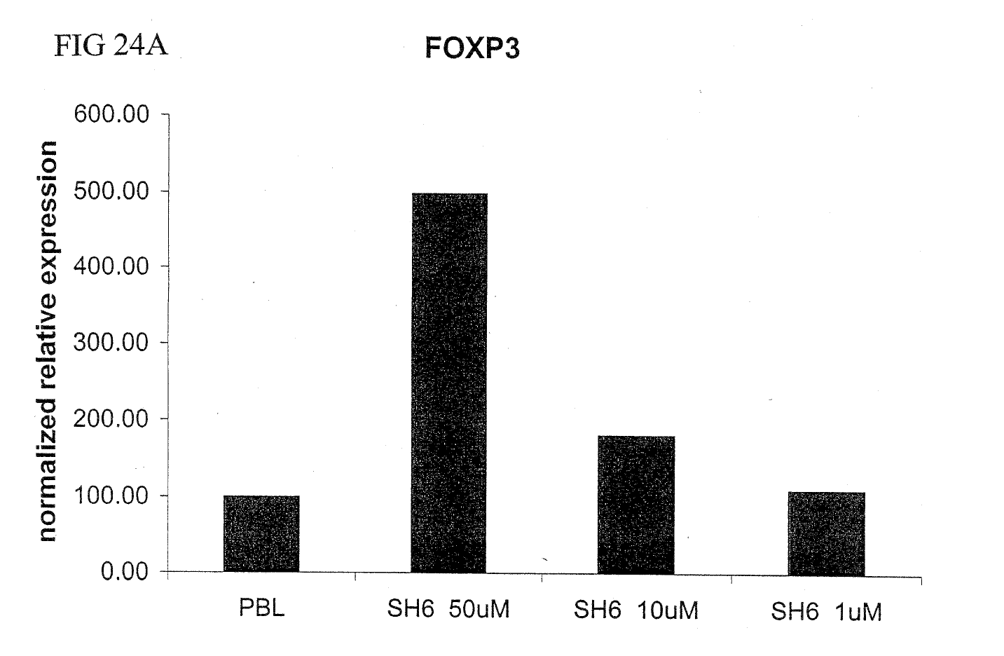

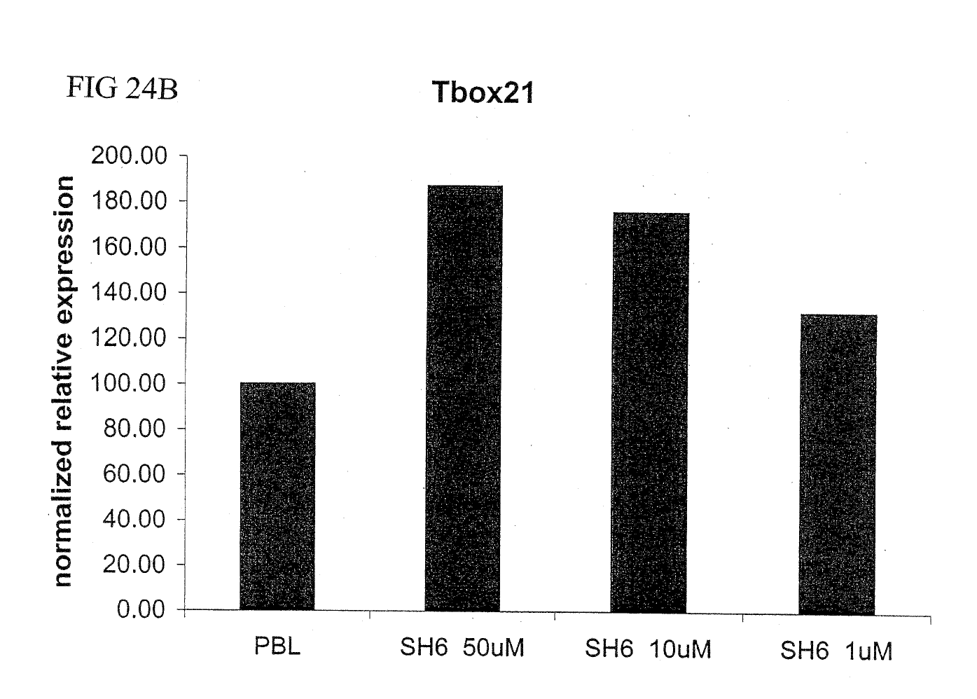

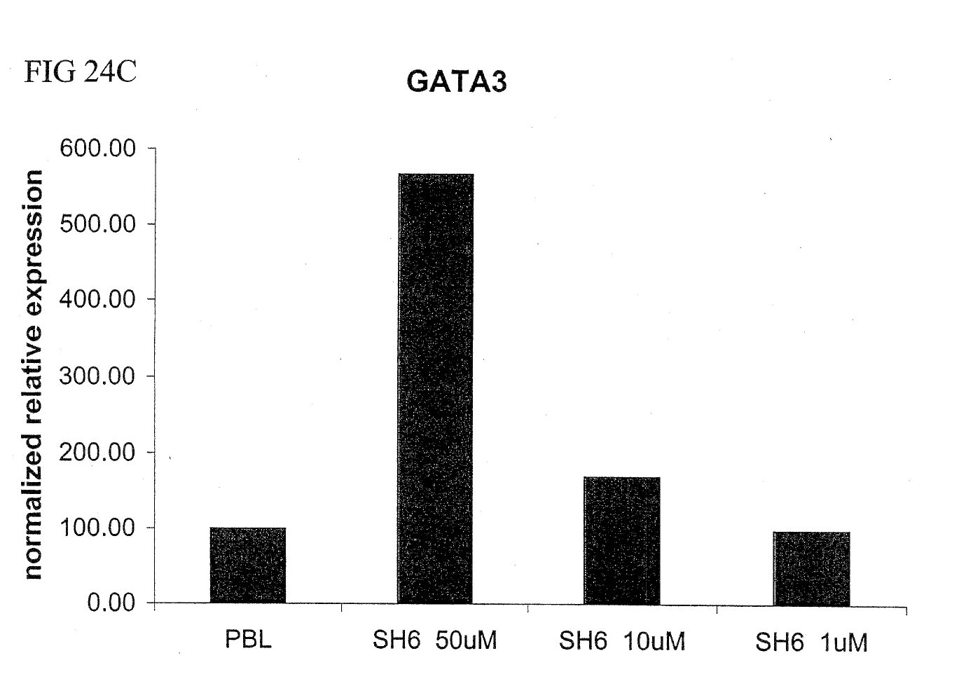

In order to dissect the impact of pathway-specific inhibitors, the changes in the expression of transcription factors has been assessed As demonstrated PBL grown in the presence of LY294002 (FIGS. 23A, 23B, and 23C) and SH-6 (FIGS. 24A, 24B, and 24C) showed significant up-regulation of specific T cell transcription factors: FOXP3 (FIGS. 23A and 24A), Tbox21 (FIGS. 23B and 24B) and GATA3 (FIGS. 23C and 24C). Importantly the magnitude of changes was identical for both inhibitors.

The data demonstrate that PI3-kinase is a dominant pathway for the regulatory T cell as assessed by the proliferation assay. In addition, Tyrosine phosphorylation of Lck, the initiator for TCR signaling is sensitive to both inhibitors, however only within the Treg subpopulation (not TH1 and TH2 cells).

The data also show that upon TCR activation the LY294002 and SH-6 impacted tyrosine-phosphorylation profile is different, but consistent for each T cell subpopulation. Expression of FOXP3, Tbox21 and GATA3 transcription factors are significantly enhanced in the human PBL culture in the presence of LY294002 and SH-6.

### Equivalents

Those skilled in the art will recognize, or be able to ascertain using no more than routine experimentation, many equivalents to the specific embodiments of the invention described herein. Such equivalents are intended to be encompassed by the following claims.

TABLE 1
Genes Preferentially Expressed in Effector (Th1 and Th2) T Cells

| | Gene | | Protein | Gi: | SEQ |
|---|---|---|---|---|---|
| Description | Name | Aliases | Product | Number | ID NO: |
| Prostaglandin E | PTGER2 | EP2; Prostaglandin E2 | PTGER2 | 31881630 | 37 and 38 |
| Receptor 2 | | receptor, EP2 subtype; | | | |
| (Subtype EP2) | | Prostanoid EP2 receptor; | | | |
| | | PGE receptor, EP2 subtype | | | |
| Transforming Growth | TGFβ1 | TGF-beta 1; CED; DPD1; | TGFβ1 | 10863872 | 39 and 40 |
| Factor, beta 1 | or | HGNC: 2997; | or | | |
| | TGFb | Transforming growth | TGFB | | |
| | | factor beta 1 precursor; | | | |
| | | TGF-beta1 | | | |

TABLE 2
Genes Preferentially Expressed in Regulatory T Cells

| | Gene | | Protein | Gi: | SEQ |
|---|---|---|---|---|---|
| Description | Name | Aliases | Product | Number | ID NO: |
| Pregnancy Specific | PSG1 | B1G1; CD66f; PSBG1; | PSG1 | 21361391 | 25 and 26 |
| Beta-1-Glycoprotein | | PSGGA; SP1; Pregnancy- | | | |
| 1 | | specific beta-1-glycoprotein | | | |
| | | 1 precursor (PSBG-1; | | | |
| | | Pregnancy-specific beta-1 | | | |
| | | glycoprotein C/D; PS-beta- | | | |
| | | C/D; Fetal liver non-specific | | | |
| | | cross-reactive antigen-2; FL- | | | |
| | | NCA-2; PSG95 | | | |
| Pregnancy Specific | PSG3 | Pregnancy-specific beta-1- | PSG3 | 11036637 | 27 and 28 |
| Beta-1-Glycoprotein | | glycoprotein 3 precursor; | | | |
| 3 | | PSBG-3); Carcinoembryonic | | | |
| | | antigen SG5 | | | |
| Pregnancy Specific | PSG6 | CGM3; PSG10; PSGGB; | PSG6 | 7524013 | 29 and 30 |
| Beta-1-Glycoprotein | | Pregnancy-specific beta-1- | | | |
| 6 | | glycoprotein 6 precursor; | | | |
| | | PSBG-6 | | | |
| Pregnancy Specific | PSG9 | PSG11; Pregnancy-specific | PSG9 | 21314634 | 31 and 32 |
| Beta-1-Glycoprotein | | beta-1-glycoprotein-11; | | | |
| 9 | | Pregnancy-specific beta-1- | | | |
| | | glycoprotein 4 precursor; | | | |
| | | PSBG-4; PSBG-9 | | | |
| jagged 1 | JAG1 | AGS; AHD; AWS; HJ1; | JAG1 | 4557678 | 1 and 2 |
| | | JAGL1; ToF; Alagille | | | |
| | | syndrome; Jagged 1 | | | |
| | | precursor; hJ1 | | | |
| G protein-coupled | GPR32 | Probable G protein-coupled | GPR32 | 4504092 | 3 and 4 |
| receptor 32 | | receptor GPR32 | | | |
| CD83 antigen | CD83 | BL11; BL11-PEN; HB15; | CD83 | 24475618 | 5 and 6 |
| | | B-cell activation, 45 kDa | | | |
| | | cell-surface glycoprotein, Ig | | | |
| | | superfamily; CD83 | | | |
| | | ANTIGEN PRECURSOR; | | | |
| | | cell-surface glycoprotein; | | | |
| | | CD83 antigen precursor; | | | |
| | | Cell surface protein HB15; | | | |
| | | B-cell activation protein | | | |
| leukocyte | CD84 | LY9B; CD84 antigen; | CD84 | 6650105 | 7 and 8 |
| differentiation | | leukocyte antigen; | | | |
| antigen CD84 | | leukocyte antigen CD84 | | | |
| isoform CD84c | | | | | |
| CD84 mRNA, | CD84 | LY9B; CD84 antigen; | CD84 | 4100318 | |
| alternatively spliced | | leukocyte antigen; leukocyte | | | |
| | | antigen CD84 | | | |
| leukocyte | CD84 | LY9B; CD84 antigen; | CD84 | 6650109 | |
| differentiation | | leukocyte antigen; | | | |
| antigen CD84 | | leukocyte antigen CD84 | | | |
| isoform CD84d | | | | | |
| leukocyte | CD84 | LY9B; CD84 antigen; | CD84 | 6650107 | |
| differentiation | | leukocyte antigen; leukocyte | | | |
| antigen CD84 | | antigen CD84 | | | |
| isoform CD84a | | | | | |
| leukocyte | CD84 | LY9B; CD84 antigen; | CD84 | 6650111 | |
| differentiation | | leukocyte antigen; leukocyte | | | |
| antigen CD84 | | antigen CD84 | | | |
| isoform CD84s | | | | | |
| Fc fragment of IgA, | FCAR | CD89; IgA Fc receptor; | FCAR | 19743864 | 9 and 10 |
| receptor for (FCAR), | | Immunoglobulin alpha Fc | | | |
| transcript variant 6 | | receptor precursor; IgA Fc | | | |
| | | receptor); CD89 antigen | | | |
| Fc fragment of IgA, | FCAR | CD89; IgA Fc receptor; | FCAR | 19743868 | |
| receptor for (FCAR), | | Immunoglobulin alpha Fc | | | |
| transcript variant 8 | | receptor precursor; IgA Fc | | | |
| | | receptor); CD89 antigen | | | |
| Fc fragment of IgA, | FCAR | CD89; IgA Fc receptor; | FCAR | 19743856 | |
| receptor for (FCAR), | | Immunoglobulin alpha Fc | | | |
| transcript variant 2 | | receptor precursor; IgA Fc | | | |
| | | receptor); CD89 antigen | | | |
| Fc fragment of IgA, | FCAR | CD89; IgA Fc receptor; | FCAR | 19743855 | |
| receptor for (FCAR), | | Immunoglobulin alpha Fc | | | |
| transcript variant 1 | | receptor precursor; IgA Fc | | | |
| | | receptor); CD89 antigen | | | |
| Fc fragment of IgA, | FCAR | CD89; IgA Fc receptor; | FCAR | 19743866 | |
| receptor for (FCAR), | | Immunoglobulin alpha Fc | | | |
| transcript variant 7 | | receptor precursor; IgA Fc | | | |
| | | receptor); CD89 antigen | | | |
| Fc fragment of IgA, | FCAR | CD89; IgA Fc receptor; | FCAR | 19743860 | |
| receptor for (FCAR), | | Immunoglobulin alpha Fc | | | |
| transcript variant 4 | | receptor precursor; IgA Fc | | | |
| | | receptor); CD89 antigen | | | |
| Fc fragment of IgA, | FCAR | CD89; IgA Fc receptor; | FCAR | 19743862 | |
| receptor for (FCAR), | | Immunoglobulin alpha Fc | | | |
| transcript variant 5 | | receptor precursor; IgA Fc | | | |
| | | receptor); CD89 antigen | | | |
| Fc fragment of IgA, | FCAR | CD89; IgA Fc receptor; | FCAR | 19743858 | |
| receptor for (FCAR), | | Immunoglobulin alpha Fc | | | |
| transcript variant 3 | | receptor precursor; IgA Fc | | | |
| | | receptor); CD89 antigen | | | |
| Fc fragment of IgA, | FCAR | CD89; IgA Fc receptor; | FCAR | 19743872 | |
| receptor for (FCAR), | | Immunoglobulin alpha Fc | | | |
| transcript variant 10 | | receptor precursor; IgA Fc | | | |
| | | receptor); CD89 antigen | | | |
| Fc fragment of IgA, | FCAR | CD89; IgA Fc receptor; | FCAR | 19743870 | |
| receptor for (FCAR), | | Immunoglobulin alpha Fc | | | |
| transcript variant 9 | | receptor precursor; IgA Fc | | | |
| | | receptor); CD89 antigen | | | |
| 5-hydroxytryptamine | HTR3A | 5-HT3R; 5HT3R; HTR3; 5- | HTR3A | 4504542 | 11 and 12 |
| (serotonin) receptor | | hydroxytryptamine | | | |
| 3A | | (serotonin) receptor 3; 5- | | | |
| | | hydroxytryptamine | | | |
| | | (serotonin) receptor-3; | | | |
| | | 5-hydroxytryptamine 3 | | | |
| | | receptor precursor; 5-HT-3; | | | |
| | | Serotonin-gated ion channel | | | |
| | | receptor; 5-HT3R | | | |
| natural killer cell | BY55 | CD160; NK1; NK28; | BY55 | 5901909 | 13 and 14 |
| receptor, | | CD160 antigen precursor; | | | |
| immunoglobulin | | Natural killer cell receptor | | | |
| superfamily member | | BY55 | | | |
| 5-hydroxytryptamine | HTR2C | HTR1C; 5- | HTR2C | 4504540 | 15 and 16 |
| (serotonin) receptor | | hydroxytryptamine 2C | | | |
| 2C | | receptor; 5-HT-2C | | | |
| | | (Serotonin) receptor; 5HT- | | | |
| | | 1C | | | |
| G protein-coupled | GPR63 | PSP24(beta); PSP24B; brain | GPR63 | 13540556 | 17 and 18 |
| receptor 63 | | expressed G-protein-coupled | | | |
| | | receptor PSP24 beta; | | | |
| | | Probable G protein-coupled | | | |
| | | receptor GPR63; PSP24- | | | |
| | | beta; PSP24-2 | | | |
| histamine receptor | HRH4 | AXOR35; BG26; GPCR105; | HRH4 | 14251204 | 19 and 20 |
| H4 | | GPRv53; H4; H4R; HH4R; | | | |
| | | GPRv53; G protein-coupled | | | |
| | | receptor 105; GPCR105; | | | |
| | | SP9144; AXOR35 | | | |
| G protein-coupled | GPR58 | phBL5 | GPR58 | 7657141 | 21 and 22 |
| receptor 58 | | | | | |
| erythropoietin | EPOR | Erythropoietin receptor | EPOR | 4557561 | 23 and 24 |
| receptor | | precursor; EPO-R | | | |
| phosphodiesterase | PDE4D | DPDE3; Phosphodiesterase- | PDE4D | 32306512 | 35 and 36 |
| 4D, cAMP-specific | | 4D, cAMP-specific (dunce | | | |
| | | (Drosophila)-homolog; | | | |
| | | phosphodiesterase 4D, | | | |
| | | cAMP-specific (dunce | | | |
| | | (Drosophila)-homolog | | | |
| | | phosphodiesterase E3); | | | |
| | | phosphodiesterase 4D, | | | |
| | | cAMP-specific | | | |
| | | (phosphodiesterase E3 dunce | | | |
| | | homolog, Drosophila); | | | |
| | | cAMP-specific 3′,5′-cyclic | | | |
| | | phosphodiesterase 4D; | | | |
| | | DPDE3; PDE43 | | | |
| PI-3-kinase-related | SMG1 | ATX; KIAA0421; LIP; | SMG1 | 18765738 | 33 and 34 |
| kinase | | lambda/iota protein kinase | | | |
| SMG-1 | | C-interacting protein; | | | |
| | | phosphatidylinositol 3- | | | |
| | | kinase-related protein kinase | | | |

What is claimed is:

1. A method for treating a subject having a condition that would benefit from modulating the balance of regulatory T cell function relative to effector T cell function in the subject, comprising administering an agent that modulates the expression or activity of a molecule selected from the group consisting of: PTGER2 and TGFβ1 to the subject such that treatment occurs.

2. A method for treating a subject having a condition that would benefit from modulating the balance of effector T cell function relative to regulatory T cell function in the subject, comprising administering an agent that modulates the expression or activity of a molecule selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase to the subject such that treatment occurs.

3. The method of claim 1 or 2, wherein the molecule is a gene and expression of the gene is downmodulated.

4. The method of claim 1 or 2, wherein the molecule is a polypeptide and activity of the polypeptide is downmodulated.

5. The method of claim 1 or 2, wherein the molecule is a gene and expression of the gene is upmodulated.

6. The method of claim 1 or 2, wherein the molecule is a polypeptide and activity of the polypeptide is upmodulated.

7. The method of claim 1 or 2, wherein effector T cell function is inhibited in said subject relative to regulatory T cell function.

8. The method of claim 7, wherein the condition is selected from the group consisting of: a transplant, an allergic response, and an autoimmune disorder.

9. The method of claim 1 or 2, wherein effector T cell function is stimulated in said subject relative to regulatory T cell function.

10. The method of claim 9, wherein the condition is selected from the group consisting of: a viral infection, a microbial infection, a parasitic infection and a tumor.

11. A method for modulating regulatory T cell function relative to effector T cell function in a population of immune cells comprising effector T cells and regulatory T cells contacting the population of cells with an agent that modulates the expression or activity of a molecule selected from the group consisting of: PTGER2 and TGFβ1 in at least a fraction of the immune cells such that regulatory T cell function relative to effector T cell function is modulated.

12. A method for modulating effector T cell function relative to regulatory T cell function in a population of immune cells comprising effector T cells and regulatory T cells contacting the population of cells with an agent that modulates the expression or activity of a molecule selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase in at least a fraction of the immune cells such that regulatory T cell function relative to effector T cell function is modulated.

13. The method of claim 11 or 12, wherein the molecule is a gene and expression of the gene is downmodulated.

14. The method of claim 11 or 12, wherein the molecule is a polypeptide and activity of the polypeptide is downmodulated.

15. The method of claim 11 or 12, wherein the molecule is a gene and expression of the gene is upmodulated.

16. The method of claim 11 or 12, wherein the molecule is a polypeptide and activity of the polypeptide is upmodulated.

17. The method of claim 11 or 12, wherein effector T cell function is inhibited in said subject relative to regulatory T cell function.

18. The method of claim 17, wherein the condition is selected from the group consisting of: a transplant, an allergic response, and an autoimmune disorder.

19. The method of claim 11 or 12, wherein effector T cell function is stimulated in said subject relative to regulatory T cell function.

20. The method of claim 19, wherein the condition is selected from the group consisting of: a viral infection, a microbial infection, a parasitic infection and a tumor.

21. An assay for identifying compounds that modulate at least one regulatory T cell function relative to modulating at least one effector T cell function comprising:
i) contacting an indicator composition comprising a polypeptide selected from the group consisting of: PTGER2 and TGFβ1 with each member of a library of test compounds;
ii) determining the ability of the test compound to modulate the activity of the polypeptide, wherein modulation of the activity of the polypeptide indicates that the test compound modulates at least one regulatory T cell function relative to at least one effector T cell function; and
iii) selecting from the library a compound of interest.

22. An assay for screening compounds that modulate at least one effector T cell function relative to modulating at least one regulatory T cell function comprising:
i) contacting an indicator composition comprising a polypeptide selected from the group consisting of: Jagged-1, GPR-32, CD83, CD84, CD89, serotonin R, BY55, serotonin R2C, GPR63, histamine R-H4, GPR58, EPO-R, PSG-1, PSG-3, PSG-6, PSG-9, PDE-4d, and PI-3-related kinase with a test compound;
ii) determining the ability of the test compound to modulate the activity of the polypeptide, wherein modulation of the activity of the polypeptide indicates that the test compound modulates at least one effector T cell function relative to at least one regulatory T cell function; and
iii) selecting from the library a compound of interest.

23. The method of claim 21 or 22, further comprising determining the effect of the compound of interest on at least one T regulatory cell function and at least one T effector cell function in an in vitro or in vivo assay.

24. The method of claim 21 or 22, wherein the indicator composition is a cell expressing the polypeptide.

25. The method of claim 23, wherein the cell has been engineered to express the polypeptide by introducing into the cell an expression vector encoding the polypeptide.

26. The method of claim 23, wherein the indicator composition is a cell that expresses the polypeptide and a target molecule, and the ability of the test compound to modulate the interaction of the polypeptide with the target molecule is monitored.

27. The method of claim 21 or 22, wherein the indicator composition comprises an indicator cell, wherein the indicator cell comprises the polypeptide and a reporter gene sensitive to activity of the polypeptide.

28. The method of claim 21 or 22, wherein the indicator composition is a cell free composition.
